(19) United States
(12) Patent Application Publication
(10) Pub. No.: US 2024/0299265 A1
(43) Pub. Date: Sep. 12, 2024

(54) ETHER COMPOUNDS AS VOLATILES FOR THE TREATMENT OF AMINO ACID BASED SUBSTRATES, SUCH AS SKIN AND HAIR

(71) Applicant: MOMENTIVE PERFORMANCE MATERIALS GMBH, Leverkusen (DE)

(72) Inventors: Roland WAGNER, Bonn (DE); Christian WENSKE, Solingen (DE); Felix NEUMEYER, Wõllstein (DE); Justine TAXACHER, Leichlingen (DE); Katharina STREICHER, Leverkusen (DE); Thorsten FELDER, Düsseldorf (DE); Mathiselvam MANOHARAN, Tamilnadu (IN); Amar PAWAR, Briarcliff Manor, NY (US); Benjamin FALK, Yorktown Heights, NY (US); Janet DUFFY, Wappingers Falls, NY (US); Kalyanachakravarthi KOTHAPALLI, Bangalore (IN)

(21) Appl. No.: 18/271,169

(22) PCT Filed: Jan. 12, 2022
(86) PCT No.: PCT/EP2022/050525

§ 371 Date: Jul. 6, 2023

(30) Foreign Application Priority Data
Jan. 12, 2021 (EP) 21151102.7

(51) Int. Cl. A61K 8/33; A61Q 1/06; A61Q 1/10; A61Q 1/14; A61Q 5/12; A61Q 7/00; A61Q 15/00; A61Q 17/04; A61Q 19/00

(52) U.S. Cl. CPC A61K 8/33; A61Q 1/06; A61Q 1/10; A61Q 1/14; A61Q 5/12; A61Q 7/00; A61Q 15/00; A61Q 17/04; A61Q 19/00; A61K 2800/10

(57) ABSTRACT

The present invention relates to a compound for use in cosmetic compositions of the formula (I): R¹—O—R², wherein R¹ and R are different from each other and are independently selected from the group of C1-C17 hydrocarbyl residues, said hydrocarbyl residues contain up to 8 CH₃ groups and up to 3 ether groups (—O—), and wherein the total number of carbon atoms of said compounds is 10 to 20, the total number of ether groups of said compounds is 1 to 6, and the total number of methyl groups (—CH₃) of said compounds CN is 1 to 13, at least one of R¹ and R² is a branched C3-C17 hydrocarbyl residue, in particular, as so-called volatiles in skin care and hair care compositions, as well as cosmetic compositions comprising at least one of said ether compounds, and the use of the aforementioned ether compounds in cosmetic compositions.

### FIELD OF THE INVENTION

This invention relates to ether compounds which are used in cosmetic compositions, in particular, as volatiles in cosmetic compositions for the treatment of amino acid based substrates, such as skin and hair.

### BACKGROUND OF THE INVENTION

Volatiles are used in a broad variety of cosmetic formulations, in particular in formulations for skin care, sun care, color cosmetics, hair care and antiperspirants/deodorants (AP deos). These formulations differ with respect to the key attributes to be addressed by the volatile. For sun care and color cosmetic formulations volatility is the key attribute. For skin care formulations, in particular soft feel is an additional important attribute. For hair care formulations, rapid spreading is an additional key parameter. In the context of AP deo formulations, a non-tackiness is to be addressed.

Cyclic, linear and branched silicones, such as decamethyl cyclopentasiloxane (D5), dodecamethyl cyclohexasiloxane (D6), short chain linear silicones having viscosities ranging from 0.5 to 3 mPa-s, as well as permethylated branched tetrasiloxanes are used as volatiles and can impart additional attributes, such as soft feel, spreadability and non-tackiness. Alternatively, hydrocarbon-based volatiles have been proposed. Typically, they belong to the groups of linear or branched hydrocarbons, fatty acid or fatty alcohol-based esters and symmetric linear ethers. US 2017/0143616 describes anhydrous cosmetic formulations containing volatiles belonging to the groups of linear C7-C17 alkanes, especially linear C11-C13 alkanes, esters of C8-C30 fatty acids with Guerbet alcohols and symmetric/asymmetric ethers of C6-C20 fatty alcohols. Isododecane (IDD; 2,2,4,6,6-pentamethyl heptane) is an example for a branched hydrocarbon used as volatile (WO2017/220743).

Compared to the silicone volatiles, the above outlined hydrocarbon-based volatiles suffer from various deficits. Linear and branched hydrocarbons are inferior with respect to the hand feel and suffer from compatibility problems with different cosmetic chassis and performance ingredients.

Fatty acid or fatty alcohol-derived esters represent high boiling compounds having a limited volatility, thus providing an oily hand feel instead of the desired light hand feel. Lower molecular weight esters tend to cause skin irritation.

Fatty alcohol derived ethers represent high boiling compounds having a limited volatility. The prior art also mentions branched ethers in cosmetic formulations.

WO2015/091380 mentions ethers having a C8-C29 fatty acid residue R¹ and a branched or unbranched C3-C30 residue R². No specific examples of such ethers are indicated.

U.S. Pat. No. 6,153,209 mentions emollients which can be ethers of C12-C18 alcohols and lower alcohols. Again, no specific examples of such ethers are indicated.

US2010/0143273 is directed at cosmetic compositions comprising at least one aprotic hydrocarbon-based volatile solvents, wherein specific C10-C13 ethers applied as such volatile solvents are disclosed.

US2013/0131188 relates to dialkyl(ene) ethers and use thereof in cosmetic preparations, wherein the ethers are restricted to such compounds containing a branched alkyl(ene) residue with 10 to 22 carbon atoms and a linear or branched alkylene residue with 1 to 13 carbon atoms.

US2007/0031361 describes waxes for cosmetic preparations. As oil component of emulsions among others, ethers, such as dialkyl ether having a total of 8 to 36 carbon atoms, in particular having 12 to 24 carbon atoms, such as, for example, di-n-octyl ether (Cetiol® OE), di-n decyl ether, di-n-nonyl ether, di-n-undecylether, di-n-dodecyl ether, di-3-ethyldecyl ether, tert-butyl n-octyl ether, isopentyl n-octyl ether, 2-methylpentyl n-octyl ether, n-hexyl n-octyl ether, n-octyl n-decyl ether, n-decyl n-undecyl ether, n-undecyl n-dodecyl ether and n-hexyl n-undecyl ether, and di-tert-butyl ether or diisopentyl ether are mentioned.

Branched ethers were also described for applications others than cosmetic formulations. Methyl-t-butyl ether (MTBE) and the ethyl-t-butyl ether (ETBE) are for example established fuel additives. t-Butyl ethers are regarded as source of highly pure isobutene (S. W. Wright et. al, Tetrahedron Lett. 1997, 38, p.7245-7248). The latter reference describes the n-decyl-t-butyl ether. Fatty alcohol-t-butyl ethers were proposed as diesel additives to reduce the NO, emissions (WO2018115574, WO2018115575). SU1142465 describes the synthesis of n-octyl-t-butyl ether from n-octanol and isobutene in the presence of a sulphonic acid modified silica catalyst.

Asymmetric branched ethers can also be synthesized by reaction of C8-C16 fatty alcohols with branched ketones and aldehydes, i.e. 4-methyl-2-pentanone, 2,2-dimethyl-propanone, 3,5,5-trimethyl hexanal, in the presence of H₂ and a Pd catalyst (Y.Fujii et. al., Bull. Chem. Soc. Japan, 2005, 78, p.456-463). The etherification of linear C1-C4 alcohols with di-isobutene was also described (R. S. Karinen et. al, Ind.Eng.Chem.Res., 2001, 40, 6073-6080; R. S. Karinen et. al., Catalysis Letters, 2001, 76(1), 81-87).

Di-t-butyl ethers are also known. Starting materials are for example ethylene glycol, propylene glycol, 1,4-butylene glycol, 1,6-hexylene glycol (S. S. Jayadeokar et. al, Reactive Polymers, 1993, 20, p.57-67; CS190755; U.S. Pat. No. 4,434,032; JP patent H0834753).

Higher branched ethers, in particular ethyl or t-butyl ethers of glycerol are of particular interest in the context of bio-fuel and fuel additives (US2011146137; S.Pariente et. al, Green Chem. 2008, 11, p.1256-1261; M.Roze et. al., Material Sci. and Appl. Chem., 2013/28, p. 67-72).

None of the above references discloses volatiles for a broad variety of cosmetic formulations, i.e. formulations for skin care, sun care, color care, hair care and AP Deos, imparting volatility in combination with other attributes, such as a light soft non-greasy hand feel, rapid spreading, non-tackiness and compatibility with a broad spectrum of cosmetic chassis and individual performance ingredients.

### SUMMARY OF THE INVENTION

The present invention relates to ether compounds which are used in cosmetic compositions, in particular, as so-called volatiles in skin care and hair care compositions, as well as cosmetic compositions comprising at least one ether compound, to novel ether compounds, and the use of the aforementioned ether compounds in cosmetic compositions and other applications.

According to the invention, it is provided a
compound for use in cosmetic compositions of the formula (I):

R¹—O—R² (I)
wherein
R¹ and R² are different from each other and are independently selected from the group of C1-C17 hydrocarbyl residues,
said hydrocarbyl residues contain up to 8 CH₃ groups and up to 3 ether groups (—O—), and wherein
the total number of carbon atoms of said compound is 10 to 20,
the total number of ether groups of said compound is 1 to 6, and
the total number of methyl groups (—CH₃) of said compound is 1 to 13,
at least one of R¹ and R² is a branched C3-C17 hydrocarbyl residue.

### DETAILED DESCRIPTION OF THE INVENTION

In the following, the invention is described in detail.

According to the present invention, it is provided a compound for use in cosmetic compositions of the formula (I):

R¹—O—R² (I)
wherein
R¹ and R² are different from each other and are independently selected from the group of C1-C17 hydrocarbyl residues,
said hydrocarbyl residues contain up to 8 CH₃ groups and up to 3 ether groups (—O—), and wherein
the total number of carbon atoms of said compound is 10 to 20,
the total number of ether groups of said compound is 1 to 6, and
the total number of methyl groups (—CH₃) of said compounds is 1 to 13,
at least one of R¹ and R² is a branched C3-C17 hydrocarbyl residue,
with the proviso for all monoether compounds containing a total number of one ether group and containing a total number of 10 to 13 carbon atoms that the compounds are selected from the group consisting of 3,5,5-trimethyl-hexyl methyl ether, 3,5,5-trimethyl-hexyl ethyl ether, 3,5,5-trimethyl-hexyl n-propyl ether, 3,5,5-trimethyl-hexyl isopropyl ether, 3,5,5-trimethyl-hexyl 2-butyl ether, 3,5,5-trimethyl-hexyl isobutyl ether and 3,5,5-trimethyl-hexyl tert-butyl ether, n-pentyl-1,1,3,3-tetramethyl-butyl ether, n-butyl-1,1,3,3-tetramethyl-butyl ether, isoamyl-1,1,3,3-tetramethyl-butyl ether, isobutyl-1,1,3,3-tetramethyl-butylether,
and with the proviso for all monoether compounds containing a total number of one ether group and a total number of 14 to 20 carbon atoms that either
   both R¹ and R² are independently selected from the groups of C5-C9 hydrocarbyl residues, or
   R¹ is selected from C5-C17 linear hydrocarbyl residues and R² is selected from the group of branched C3-C9 hydrocarbyl residues, or
   one of the groups R¹ and R² is selected from linear C14-C17 hydrocarbyl residues, and with the exception of all monoether compounds containing at least one of R¹ and R² representing a 2-propyl-heptyl group.

Preferably, the compound of the formula (I) according to the invention is used in cosmetic compositions as a volatile.

According to the invention, the term “hydrocarbyl residue” as applied to R¹ and R² of formula (I) generally refers to a monovalent residue bonded to the O atom of formula (I) by a single bond between a C atom of the residue and said oxygen atom of formula (I), wherein the term “hydrocarbyl residue” is generally understood as a residue consisting of C atoms and H atoms.

It is further understood that according to the invention the groups R¹ and R² of formula (I) are separate groups, which means that R¹ and R² are not bonded or connected to each other otherwise than by the ether O-atom as displayed in formula (I).

It is also generally understood that any structure containing one or more stereogenic centers without explicit mention of stereodescriptors refers to both enantiomers or all diastereomers possible, both in practically enantiomerically or diastereomerically pure form and as enantiomeric and diastereomeric mixtures of any ratio of enantiomers or diastereomers, respectively.

By the proviso that the hydrocarbyl residue may contain up to 3 ether groups (—O—), it is defined that each of the hydrocarbyl residues R¹ and R² of formula (I) may independently contain 0, 1, 2 or three ether groups. It is noted that the expressions “hydrocarbyl group” and “hydrocarbyl residue” are used interchangeably herein and have the same meaning.

According to the invention, an ether group (—O—) is a functional group consisting of a divalent oxygen atom linking two C atoms of organyl residues, wherein according to the invention the organyl residues linked by the oxygen atom are hydrocarbyl residues consisting of carbon atoms and hydrogen atoms, or hydrocarbyl residues containing carbon atoms, hydrogen atoms and oxygen atoms.

According to the invention, the presence of other heteroatoms than oxygen in the residues R¹ and R² and thus in the compounds of formula (I) is excluded.

Likewise, according to the invention the presence of other oxygen atom-containing functional groups than one or more ether groups, such as hydroxy groups, aldehyde groups, keto groups, ester groups, hydroperoxide groups and peroxy ester groups, in the residues R¹ and R², and thus in the compounds of formula (I), is excluded.

According to the invention, the residues R¹ and R² of formula (I) are different from each other, and are independently selected from the group of C1-C17 hydrocarbyl residues, which may be C1-C17 linear, branched, cycloaliphatic or aromatic hydrocarbyl groups with up to 17 C-atoms, wherein at least one of the residues R¹ and R² is a branched acyclic hydrocarbyl residue.

Therein, the term “linear hydrocarbyl group” denotes hydrocarbyl groups as defined above in which the group's carbon atoms are exclusively linked in a continuous chain, i.e. no carbon atom of the group is bonded to more than two neighboring atoms selected from carbon atoms and oxygen atoms, and there is one terminal carbon atom bonded to one neighboring carbon atom or to one ether group oxygen atom exclusively.

The term “branched hydrocarbyl group” denotes hydrocarbyl groups as defined above in which the structure of the group has at least one branch, i.e. at least one carbon atom of the group is bonded to more than two neighboring atoms selected from carbon atoms and oxygen atoms, and there are at least two terminal carbon atom bonded to one neighboring carbon atom or to one ether group oxygen atom exclusively.

In addition to the above definition it is explicitly noted that according to the invention the term “branched hydrocarbyl group” refers to acyclic branched residues that do not contain any cycloaliphatic or aromatic structural moieties.

The term “cycloaliphatic hydrocarbyl group” denotes hydrocarbyl groups as defined above in which the structure of the group contains at least one cyclic moiety, i.e. at least one carbon atom of the group is bonded to more than two neighboring atoms selected from carbon atoms and oxygen atoms, while the number of terminal carbon atom bonded to one neighboring carbon atom or to one ether group oxygen atom exclusively is lower than expected in view of the number of branches of the hydrocarbyl scaffold.

It is noted that a hydrocarbyl group that contains both cyclic aliphatic structural moieties and branched acyclic moieties or linear moieties is considered as a cycloaliphatic hydrocarbyl group according to the invention.

The term “aromatic hydrocarbyl group” denotes hydrocarbyl groups as defined above that contain at least one cyclically conjugated molecular entity with a stability which due to delocalization is significantly greater than that of a hypothetical localized structure, for example a phenyl or furan moiety.

It is noted that every hydrocarbyl group that contains such aromatic cyclic structure is considered as an aromatic hydrocarbyl structure regardless if it also comprises one or more linear, branched or cycloaliphatic moieties.

According to the invention, the term “linear, branched or cycloaliphatic hydrocarbyl groups” comprises linear, branched or cyclic alkyl groups, alkene groups or alkyne groups, wherein the alkene groups may comprise one or more C—C double bonds, and the alkyne groups may comprise one or more C—C triple bonds. It is preferred that at least one of R¹ and R² is independently selected from a linear, branched or cycloaliphatic alkyl group, wherein the chain of carbon atoms may be generally interrupted by one or more ether group —O— atoms, as described above.

Examples of linear alkyl groups are the C1-C17 n-alkyl groups methyl, ethyl, n-propyl, n-butyl, n-pentyl, n-hexyl, n-heptyl or n-octyl, n-nonyl, n-decyl, n-undecyl, n-dodecyl, n-tridecyl, n-tetradecyl, n-pentadecyl, n-hexadecyl or n-heptadecyl groups.

Examples of branched alkyl groups are the C3-C17 alkyl groups containing at least one branch iso-propyl, 2-methyl-propyl, 2-methyl-butyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, and 2-ethyl-hexyl, wherein t-butyl, 1,1,3,3-tetramethyl-butyl, 2,4,4-trimethylpentyl, 3,5,5-trimethylhexyl and 1,1,3,3,5,5-hexamethyl-hexyl are preferred.

Examples of cyclic alkyl groups are the cyclic C5-C17 alkyl groups containing at least one cyclic moiety cyclopentyl, cyclohexyl, cycloheptyl, 1-methyl-cyclohexyl, 2-methyl-cyclohexyl, 3-methyl-cyclohexyl, 4-methyl-cyclohexyl, all dimethyl-cyclohexyl regioisomers, 1-isopropyl-cyclohexyl, 2-isopropyl-cyclohexyl, 3-isopropyl-cyclohexyl, 4-isopropyl-cyclohexyl, 1-t-butyl-cyclohexyl, 2-t-butyl-cyclohexyl, 3-t-butyl-cyclohexyl, 4-t-butyl-cyclohexyl, 2,4-di-tert-butyl-cyclohexyl, 2,6-di-tert-butyl-cyclohexyl, all isopropyl-methyl-cyclohexyl regioisomers, wherein cyclohexyl and cyclopentyl are preferred.

According to the invention, the term “aromatic alkyl group” is used for any group comprising an aromatic group and no further groups except alkylene and alkyl groups, i.e. saturated hydrocarbon groups, wherein both alkylene and alkyl groups may contain one or more ether oxygen atoms.

Examples of aromatic alkyl groups are the aromatic C6-C12 groups benzyl, 4-methylbenzyl, 2-methylphenyl, 3-methylphenyl, 4-methylphenyl, wherein 4-methylphenyl is preferred.

Examples of linear alkyl groups containing one —O— ether group are the C3-C17 groups having a structure of the formula R³—O—CH₂CH₂—, wherein R³ is a linear C1-C15 alkyl group selected from methyl, ethyl, n-propyl, n-butyl, n-pentyl, n-hexyl, n-heptyl or n-octyl, n-nonyl, n-decyl, n-undecyl, n-dodecyl, n-tridecyl, n-tetradecyl or n-pentadecyl.

Examples of branched alkyl groups containing one —O— ether group are the C3-C17 groups selected from the structures of the formulas R⁴—O—CH₂CH(CH₃)—, R⁵—O—CH₂CH(CH₂CH₃)—, R⁶—O—CH₂CH₂CH(CH₃)—, R⁸—O—CH₂CH(CH₃)—, R⁹—O—CH₂CH(CH₂CH₃)— or R¹⁰—O—CH₂CH₂CH(CH₃)—,
wherein R⁴, R⁵, R⁶, R⁸, R⁹ and R¹⁰ are selected from the group consisting of linear C1-C14 alkyl groups, in particular methyl, ethyl, n-propyl, n-butyl, n-pentyl, n-hexyl, n-heptyl or n-octyl, n-nonyl, n-decyl, n-undecyl, n-dodecyl, n-tridecyl and n-tetradecyl,
and branched C3-C14 alkyl groups, in particular iso-propyl, 2-methyl-propyl, 2-methyl-butyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, and 2-ethyl-hexyl,
or R⁷—O—CH₂CH₂—, wherein R⁷ is selected from C3-C15 branched alkyl residues, in particular iso-propyl, 2-methyl-propyl, 2-methyl-butyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, and 2-ethyl-hexyl.

Examples of cyclic alkyl groups containing one —O— ether group are the C7-C17 groups selected from the structures of the formulas R³—O—CH₂CH₂—, R⁴—O—CH₂CH(CH₃)—, R⁵—O—CH₂CH(CH₂CH₃)—, R⁶—O—CH₂CH₂CH(CH₃)—, R⁷—O—CH₂CH₂—, R⁸—O—CH₂CH(CH₃)—, R⁹—O—CH₂CH(CH₂CH₃)— or R¹⁰—O—CH₂CH₂CH(CH₃)—, wherein R³, R⁴, R⁵, R⁶, R⁸, R⁹ and R¹⁰ are selected from the group consisting of cyclic C5-C17 alkyl groups, in particular cyclopentyl, cyclohexyl, cycloheptyl, 1-methyl-cyclohexyl, 2-methyl-cyclohexyl, 3-methyl-cyclohexyl, 4-methyl-cyclohexyl, all dimethyl-cyclohexyl regioisomers, 1-isopropyl-cyclohexyl, 2-isopropyl-cyclohexyl, 3-isopropyl-cyclohexyl, 4-isopropyl-cyclohexyl, 1-t-butyl-cyclohexyl, 2-t-butyl-cyclohexyl, 3-t-butyl-cyclohexyl, 4-t-butyl-cyclohexyl, 2,4-di-tert-butyl-cyclohexyl, 2,6-di-tert-butyl-cyclohexyl, all isopropyl-methyl-cyclohexyl regioisomers, wherein cyclopentyl, cyclohexyl and methyl cyclohexyl groups are preferred.

Examples of aromatic alkyl groups containing one —O— ether group are the C6-C17 groups selected from the structures of the formulas R³—O—CH₂CH₂—, R⁴—O—CH₂CH(CH₃)—, R⁵—O—CH₂CH(CH₂CH₃)—, R⁶—O—CH₂CH₂CH(CH₃)—, R⁷—O—CH₂CH₂—, R⁸—O—CH₂CH(CH₃)—, R⁹—O—CH₂CH(CH₂CH₃)— or R¹⁰—O—CH₂CH₂CH(CH₃)—,
wherein R³, R⁴, R⁵, R⁶, R⁸, R⁹ and R¹⁰ are selected from the group consisting of aromatic C4-C15 groups and C5-C15 aromatic alkyl groups, in particular phenyl, 2-furyl, benzyl, 4-methylbenzyl, 2-methylphenyl, 3-methylphenyl and 4-methylphenyl groups, wherein the 4-methylphenyl group is preferred.

Examples of linear alkyl groups containing two —O— ether groups are the C5-C17 alkyl groups having a structure of the formula R¹¹—O—CH₂CH₂—O—CH₂CH₂—, wherein R¹¹ is selected from linear C1-C13 alkyl groups, preferably C1-C12 alkyl groups alkyl group selected from methyl, ethyl, n-propyl, n-butyl, n-pentyl, n-hexyl, n-heptyl or n-octyl, n-nonyl, n-decyl, n-undecyl or n-dodecyl.

Examples of branched alkyl groups containing two —O— ether groups are the C7-C17 groups selected from the structures of the formulas R¹²—O—CH₂CH(CH₃)—O—CH₂CH(CH₃)—, R¹³—O—CH₂CH(CH₂CH₃)—O—CH₂CH(CH₂CH₃)—, R¹⁵—O—CH₂CH(CH₃)—O—CH₂CH(CH₃)—, R¹⁶—O—CH₂CH(CH₂CH₃)—O—CH₂CH(CH₂CH₃)—, R¹⁷—O—CH₂CH(—OR¹⁸)CH₂—, wherein the residues R¹², R¹³, R¹⁵, R¹⁶, R¹⁷ and R¹⁸ are selected from linear C1-C13 alkyl groups, in particular methyl, ethyl, n-propyl, n-butyl, n-pentyl, n-hexyl, n-heptyl or n-octyl, n-nonyl, n-decyl, n-undecyl or n-dodecyl groups,
or from branched C3-C13 alkyl groups, in particular iso-propyl, 2-methyl-propyl, 2-methyl-butyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, and 2-ethyl-hexyl,
or of the formula R¹⁴—O—CH₂CH₂—O—CH₂CH₂—, wherein R¹⁴ is selected from C3-C13 branched alkyl residues, in particular particular iso-propyl, 2-methyl-propyl, 2-methyl-butyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, and 2-ethyl-hexyl.

Examples of cyclic alkyl groups containing two —O— ether groups are the C9-C17 groups selected from the structures of the formulas R¹²—O—CH₂CH(CH₃)—O—CH₂CH(CH₃)—, R¹³—O—CH₂CH(CH₂CH₃)—O—CH₂CH(CH₂CH₃)—, R¹⁴—O—CH₂CH₂—O—CH₂CH₂—, R¹⁵—O—CH₂CH(CH₃)—O—CH₂CH(CH₃)—, R¹⁶—O—CH₂CH(CH₂CH₃)—O—CH₂CH(CH₂CH₃)—, wherein the residues R¹², R¹³, R¹⁴ R¹⁵ and R¹⁶ are selected from C5-C13 cyclic alkyl groups, in particular cyclopentyl, cyclohexyl, cycloheptyl, 1-methyl-cyclohexyl, 2-methyl-cyclohexyl, 3-methyl-cyclohexyl, 4-methyl-cyclohexyl, all dimethyl-cyclohexyl regioisomers, 1-isopropyl-cyclohexyl, 2-isopropyl-cyclohexyl, 3-isopropyl-cyclohexyl, 4-isopropyl-cyclohexyl, 1-t-butyl-cyclohexyl, 2-t-butyl-cyclohexyl, 3-t-butyl-cyclohexyl, 4-t-butyl-cyclohexyl, 2,4-di-tert-butyl-cyclohexyl, 2,6-di-tert-butyl-cyclohexyl, all isopropyl-methyl-cyclohexyl regioisomers,
or of the formula R¹⁷—O—CH₂CH(—OR¹⁸)CH₂—, wherein at least one of R¹⁷ and R¹⁸ is selected from the C5-C13 cyclic alkyl groups disclosed above, while the R¹⁷ or R¹⁸ group not representing a cyclic alkyl group is selected from C1-C13 linear alkyl groups or C3-C13 branched alkyl groups,
wherein R¹², R¹³, R¹⁴ R¹⁵ R¹⁶, and one or both of R¹⁷ and R¹⁸ are preferably selected from cyclopentyl, cyclohexyl and methyl cyclohexyl groups.

Examples of aromatic alkyl groups containing two —O— ether groups are the C9-C17 groups selected from the structures of the formulas R¹²—O—CH₂CH(CH₃)—O—CH₂CH(CH₃)—, R¹³—O—CH₂CH(CH₂CH₃)—O—CH₂CH(CH₂CH₃)—, R¹⁴—O—CH₂CH₂—O—CH₂CH₂—, R¹⁵—O—CH₂CH(CH₃)—O—CH₂CH(CH₃)—, R¹⁶—O—CH₂CH(CH₂CH₃)—O—CH₂CH(CH₂CH₃)—, wherein the residues R¹², R¹³, R¹⁴ R¹⁵ and R¹⁶ are selected from C4-C13 aromatic groups or C5-C13 aromatic alkyl groups, in particular phenyl, 2-furyl, benzyl, 4-methylbenzyl, 2-methylphenyl, 3-methylphenyl and 4-methylphenyl groups,
or from a structure of the formula R¹⁷—O—CH₂CH(—OR¹⁸)CH₂—, wherein at least one of R¹⁷ and R¹⁸ is selected from a C4-C13 aromatic group or C5-C13 aromatic alkyl group, in particular phenyl, 2-furyl, benzyl, 4-methylbenzyl, 2-methylphenyl, 3-methylphenyl and 4-methylphenyl groups, while the R¹⁷ or R¹⁸ group not representing an aromatic group or C5-C13 aromatic alkyl group is selected from C1-C13 linear alkyl groups, C3-C13 branched alkyl groups or C5-C15 cyclic alkyl groups,
wherein R¹², R¹³, R¹⁴ R¹⁵ R¹⁶, and one or both of R¹⁷ and R¹⁸ are preferably selected from phenyl, 2-furyl and 4-methylphenyl groups.

It is thus noted that the C1-C17 hydrocarbyl groups may comprise both a cyclic structure and a linear structure, or both a cyclic structure and a branched structure, wherein in both cases the cyclic structure may be at a terminal position of the R¹ or R² residue, may be bonded to the —O— ether group linking the R¹ and the R² group, or may be positioned within the structure of R¹ or R² neither in a terminal position nor bonded to the —O— ether group linking the R¹ and the R² group of the compound.

According to the invention, aromatic hydrocarbyl groups are hydrocarbon compounds containing one or more aromatic hydrocarbon groups with a total of up to 17 C-atoms.

Therein, the carbon atom scaffold may be interrupted by ether group —O— atoms as defined above. Further, the term in particular comprises such hydrocarbyl groups that comprise one or both of a linear or branched structure, in particular a linear alkyl or alkylene group or a branched alkyl or alkylene group, or several of the aforementioned groups, and an aromatic cyclic moiety such as a phenyl ring or a furan ring.

According to the invention, each of the groups R¹ and R² may independently contain 0-8, preferred 0-7, preferred 0-6, preferred 0-5, preferred 0-3, or preferred 1-8, more preferred 1-7, even more preferred 1-6, further preferred 1-5, even further preferred 1-3, specifically preferred 1, 2, 3, 4, 5, 6, or 7 CH₃ groups.

Also according to the invention, each of the groups R¹ and R² may independently contain 1-3, preferably 1-2, specifically preferred 1 or 2 oxygen atoms as ether groups.

The compounds of the formula (I) may in total contain 1, 2, 3, 4, 5 or 6 ether group oxygen atoms as the sum of ether group oxygen atoms present in the groups R¹ and R². It is excluded that both groups R¹ and R² each contain three oxygen atoms, and thus the total number of oxygen atoms in the compounds of the formula (I) is at most 6. The total number of ether oxygen atoms in the compounds of the formula (I) is 1-6, preferred 1-4, more preferred 1-3, even more preferred 1-2, specifically preferred 1, 2 or 3.

Accordingly, the compounds of the formula (I) according to the invention may be selected from the group of mono-ether compounds (one ether group (—O—) per molecule), di-ether compounds (two ether groups (—O—) per molecule), tri-ether compounds (three ether groups (—O—) per molecule), tetra-ether compounds (four ether groups (—O—) per molecule), penta-ether compounds (five ether groups (—O—) per molecule) or hexa-ether compounds (six ether groups (—O—) per molecule).

According to the invention, it is preferred that the compound of the formula (I) is selected from the group of mono-ether compounds, di-ether compounds, and tri-ether compounds.

According to the invention, it is further preferred that if the compound has one ether group (—O—), at least one of R¹ and R² has at least 6 carbon atoms, or at least one of R¹ and R² is a branched alkyl group, or at least one of R¹ and R² has at least 6 carbon atoms and at least one of R¹ and R² is a branched alkyl group.

Further, according to the invention, the total number of carbon atoms of the compounds of the formula (I) is 10 to 20, i.e. E carbon atoms in R¹+R² is 10-20, preferred 10-17, more preferred 10-15, even more preferred 11-15, further preferred 11-13, specifically preferred 11, 12, 13, 14, or 15. It is thus excluded that the sum of carbon atoms of R¹ and R² is 9 or less, or 21 or more.

According to the invention, the total number of methyl groups in the compounds of the formula (I) is 1 to 13, i.e. ΣCH₃ groups in R¹+R² 1-13, preferred 1-12, more preferred 1-10, even more preferred 1-9, further preferred 1-7, still further preferred 1-6, still more further preferred 1-4, or preferred 2-6, more preferred 2-4, and specifically preferred 1, 2, 3, 4, 5, 6, 7, 8, 9, 10, 11 12 or 13. It is thus excluded that none of the residues R¹ and R² contains a methyl group, e.g. it is excluded that both R¹ and R² are cycloaliphatic or aromatic groups, and it is excluded that the sum of methyl groups in R¹ and R² is 14 or more.

According to the invention, the term “cosmetic composition” refers to any kind of mixture of compounds intended to be placed in contact with the various external parts of the human body (epidermis, hair system, nails, lips and external genital organs) or with the teeth and the mucous membranes of the oral cavity with a view exclusively or mainly to cleaning them, perfuming them, changing their appearance and/or correcting body odours and/or protecting them or keeping them in good condition.

Cosmetic composition can be “leave-on”—desired to leave on the applied body part until removed or “wash-off”—intended to be washed immediately after application.

Specific examples of “leave-on” cosmetic compositions according to the invention are antiperspirant and deodorant compositions in the form of spray, pencils, sticks, multiphase sticks, pastes, powder, aerosols, creams, cream foams, lotions, self-foaming, foam-like, after-foaming or foamable emulsions, gels, roll-on preparations, foams or depilatories, skin care compositions such as lotions, creams, emulsions and microemulsions, moisturizers, anti-aging products, skin tightening lotions, anti-acne products, day-creams, night-cream, under eye-cream, face mask, face lotion, body lotion, after-shave lotions, cleaning milk, toners, color cosmetic compositions such as lip-stick, pencils, lip-color, liquid lip color, lip stain, lip balm, lip-gloss, mascara, eye liner, eye-shadow, foundation, face powder, blush, peel cream, bb cream, cc cream, tinted moisturizer, hair care compositions such as hair treatments, damage repair compositions, hair waxes, hair gels, hair foam, hair styling fluids, hair sprays, hair oil, mousse, leave-in conditioner, hair remover creams, hair coloring products, nail care compositions such as nail polish, nail enamel, top coat, cuticle softener, and sun care compositions such as sunscreens, after sun lotions, sun tanner, sunscreen oils, self-tanning products.

Specific examples of “wash-off” cosmetics according to the invention are shampoo, conditioner, bath soaps, bath gels, make up remover, toners, cleansing solutions, cleansing milk.

According to the invention, the term “volatile” when applied to a compound for the use in cosmetic compositions is defined as referring to a volatile solvent which makes it possible to bring about a change in the properties of the product comprising said volatile solvent during and after deposition, thereby resulting, depending on the cosmetic product envisaged, in properties of staying power of the deposited product, or of comfort or texture during application of the product, and also in specific mechanical or optical properties of the deposits.

Preferably, the boiling point of such volatile compound is in the range from 150° C. to 300° C., more preferably from 150° C. to 280° C., even more preferably from 150° C. to 250° C., and most preferably from 170 to 240° C.

According to the invention, when the compound is a mono-ether compound containing a total number of one ether group and containing a total number of carbon atoms of 10 to 13, the compounds for use in cosmetic compositions of the formula (I) as defined above are selected from the group consisting of 3,5,5-trimethyl-hexyl methyl ether, 3,5,5-trimethyl-hexyl ethyl ether, 3,5,5-trimethyl-hexyl n-propyl ether, 3,5,5-trimethyl-hexyl isopropyl ether, 3,5,5-trimethyl-hexyl 2-butyl ether, 3,5,5-trimethyl-hexyl isobutyl ether and 3,5,5-trimethyl-hexyl tert-butyl ether, n-pentyl-1,1,3,3-tetramethyl-butyl ether, n-butyl-1,1,3,3-tetramethyl-butyl ether, isoamyl-1,1,3,3-tetramethyl-butyl ether, isobutyl-1,1,3,3-tetramethyl-butylether.

Further, according to the invention, for all monoether compounds of the general formula (I) according to the invention containing a total number of one ether group and a total number of 14 to 20 carbon atoms at least one of the following provisos needs to be fulfilled:
both R¹ and R² are independently selected from the group of C5-C9 hydrocarbyl residues, or
R¹ is selected from C5-C17 linear hydrocarbyl residues and R² is selected from the group of branched C3-C9 hydrocarbyl residues, or
one of the groups R¹ and R² is selected from linear C14-C17 hydrocarbyl residues.

Finally, all monoether compounds containing at least one of R¹ and R² representing a 2-propyl-heptyl group are excluded from the scope of the invention.

According to the invention, the appearance of an asymmetrical linker group, such as a for example a “1,3-butylene group”, is understood to cover both orientations possible of the group within the compound containing said group, and thus to cover all of the regioisomers resulting therefrom unless otherwise noted.

This means that, for instance, a structural formula
“R¹⁹—O—R²⁰—O—R²,
wherein R²⁰ is a 1,3-butylene group” or
“R¹⁹—O—R²⁰—O—R²,
wherein R²⁰ is —CH₂CH₂C(CH₃)H—”
covers both of the structures
R¹⁹—O—CH₂CH₂C(CH₃)H—O—R² and R¹⁹—O—C(CH₃)H—CH₂—CH₂—O—R²—.

In a preferred embodiment according to the invention, the compound for use in cosmetic compositions of the formula (I) according to the invention has one ether group (—O—), i.e. it is
a mono ether compound, and
at least one of R¹ and R² has at least 6 carbon atoms, and
at least one of R¹ and R² is a branched alkyl group.

It is also preferred that the compound for use in cosmetic compositions of the formula (I) according to the invention is a mono ether compound, at least one of R¹ and R² is a branched alkyl group, and
at least one of R¹ and R² has at least 7 carbon atoms, at least 8 carbon atoms, or at least 9 carbon atoms.

It is likewise preferred that the compound for use in cosmetic compositions of the formula (I) according to the invention is a mono ether compound, both R¹ and R² are branched alkyl groups, and at least one of R¹ and R² has at least 6, at least 7, at least 8 or nine carbon atoms.

In another preferred embodiment according to the invention, the compound for use in cosmetic compositions of the formula (I) according to the invention is selected from the group of mono-ether compounds (one ether group (—O—) per molecule), di-ether compounds (two ether groups (—O—) per molecule), and tri-ether compounds (three ether groups (—O—) per molecule).

In still another preferred embodiment according to the invention, wherein at least one of R¹ and R² of the compound for use in cosmetic compositions of the formula (I) according to the invention is a branched hydrocarbyl residue and the other residue is selected from linear, branched, cycloaliphatic or aromatic hydrocarbyl groups.

Therein, it is preferred when R¹ is a branched group selected from 1,1,3,3-tetramethylbutyl and 1,1,3,3,5,5-hexamethylhexyl, or from a branched group containing one ether O-atom, and R² is selected from linear, branched or cyclic aliphatic groups, in particular from isopropyl, isobutyl, tert-butyl or isopentyl groups.

It is more preferred that R¹ is a branched group containing one ether O-atom, wherein the terminal alkyl group of the R¹ group is branched, in particular wherein the terminal alkyl group of the R¹ group is selected from an isopropyl group, an isobutyl group, a tert-butyl group, an isopentyl group, or a 1,1,3,3-tetramethylbutyl group, while R² is selected from a branched group not containing an ether oxygen atom. It is even more preferred according to this embodiment that R¹ is a branched group containing one ether O-atom, wherein the terminal alkyl group of the R¹ group is branched and R² is selected from an isopropyl, isobutyl, tert-butyl, isopentyl or 1,1,3,3-tetramethylbutyl group, and it is most preferred that R¹ is a branched group containing one ether O-atom, wherein the terminal alkyl group of the R¹ group is selected from an isopropyl group, an isobutyl group, a tert-butyl group, an isopentyl group, or a 1,1,3,3-tetramethylbutyl group, and R² is selected from an isopropyl, n-butyl, isobutyl, tert-butyl, n-pentyl, isopentyl, isoamyl, isobutyl or 1,1,3,3-tetramethylbutyl group.

In a preferred embodiment according to the invention, the number of methyl groups in R¹ and R² of the compound for use in cosmetic compositions of the formula (I) according to the invention is independently 0-7, preferred 0-6, more preferred 0-5, even more preferred 0-3, or preferred 1-7, more preferred 1-6, even more preferred 1-5, further preferred 1-3, specifically preferred 1, 2, 3, 4, 5, 6, or 7.

While the number of methyl groups of R¹ and R² may be independently selected, the overall number of methyl groups of the compound according to the invention cannot exceed the number of 13 methyl groups.

In a further preferred embodiment according to the invention, the total number of carbon atoms of the compound for use in cosmetic compositions of the formula (I) is 10-17, preferred 10-15, more preferred 11-15, even more preferred 11-13, and specifically preferred 11, 12, 13, 14, or 15.

Such number of carbon atoms provides compounds of the formula (I) displaying the appropriate degree of volatility, which is further determined by the selection of the number of branches and the number and position of ether O-atoms, if present, in the residues R¹ and R².

In another preferred embodiment according to the invention, the total number of ether groups of the compound for use in cosmetic compositions of the formula (I) is 1 to 5, preferred 1-4, more preferred 1-3, even more preferred 1-2, and specifically preferred 1, 2, or 3.

The number of O atoms is not only relevant regarding the molecular weight of the compound for use in cosmetic compositions of the formula (I), but it is also crucial for adjusting the dipole moment and the flexibility of the compounds' structures, and thus the polarity of the compounds. Therein, the position of the O atoms is also crucial.

In still another preferred embodiment according to the invention, the total number of methyl groups (—CH₃) of said compound is 1-12, preferred 1-10, more preferred 1-9, even more preferred 1-7, further preferred 1-6, even further preferred 1-4, or preferred 2-6, more preferred 2-4, and specifically preferred 1, 2, 3, 4, 5, 6, 7, 8, 9, 10, 11, or 12.

Therein, it is preferred that for the compound for use in cosmetic compositions of the formula (I) containing
one oxygen atom, the number of methyl groups is 4, 6, 8,
two oxygen atoms, the number of methyl groups is 4, 5, 6, 7,
three oxygen atoms, the number of methyl groups is 3, 6, 9.

It is particularly preferred that for the compound for use in cosmetic compositions of the formula (I) containing one oxygen atom and the number of methyl groups is 4, 6, or 8 that one of R¹ and R² is derived from isobutene, di-isobutene or tri-isobutene.

In a preferred embodiment according to the invention, the linear, branched, cycloaliphatic or aromatic hydrocarbyl residues R¹ and R² in formula (I) are independently selected from
alkyl groups, which may be selected from the group consisting of linear, branched, or cyclic alkyl groups and aromatic C1-C17 alkyl groups, in particular from
   linear C1-C17 alkyl groups, such as methyl, ethyl, n-propyl, n-butyl, n-pentyl, n-hexyl, n-heptyl or n-octyl, n-nonyl, n-decyl, n-undecyl, n-dodecyl groups,
   branched C1-C17 alkyl groups, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, 2-ethyl-hexyl,
   cyclic C5-C17 alkyl groups, such as cyclohexyl,
   arylalkyl C7-C13 groups, such as benzyl, and
   aromatic C7-C13 alkyl groups, such as, 2-methylphenyl, 3-methylphenyl, 4-methylphenyl,
aromatic C6-C12 groups, such as phenyl groups, and
one —O— ether moiety containing alkyl groups, which may be selected from the group consisting of linear, branched, cyclic and aromatic C3-C17 alkyl groups, in particular selected from
one —O— ether moiety containing linear C3-C17 alkyl groups, of the formula R³—O—CH₂CH₂—, wherein R³ is selected from linear C1-C15 alkyl groups, in particular from methyl, ethyl, n-propyl, n-butyl, n-pentyl, n-hexyl, n-heptyl or n-octyl, n-nonyl, n-decyl, n-undecyl and n-dodecyl,
one —O— ether moiety containing branched C4-C17 alkyl groups, in particular of the formula R⁴—O—CH₂CH(CH₃)—, wherein R⁴ is selected from linear C1-C14 alkyl groups, such as methyl, ethyl, n-propyl, n-butyl, n-pentyl, n-hexyl, n-heptyl or n-octyl, n-nonyl, n-decyl, n-undecyl and n-dodecyl,
of the formula R⁵—O—CH₂CH(CH₂CH₃)—, wherein R⁵ is selected from linear C1-C13 alkyl groups, such as methyl, ethyl, n-propyl, n-butyl, n-pentyl, n-hexyl, n-heptyl or n-octyl, n-nonyl, n-decyl, n-undecyl, and n-dodecyl,
of the formula R⁶—O—CH₂CH₂CH(CH₃)—, wherein R⁶ is selected from is selected from linear C1-C13 alkyl groups, such as methyl, ethyl, n-propyl, n-butyl, n-pentyl, n-hexyl, n-heptyl or n-octyl, n-nonyl, n-decyl, n-undecyl, and n-dodecyl,
one —O— ether moiety containing branched, cyclic or aromatic C4-C17 alkyl groups of the formula R⁷—O—CH₂CH₂—, wherein R⁷ is selected from C3-C15 branched or cyclic aliphatic residues or aromatic residues, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, 2-ethyl-hexyl, C5-C15 cyclic alkyl residues, such as cyclohexyl, and C6-C12 aromatic residues, such as phenyl, 2-methylphenyl, 3-methylphenyl, 4-methylphenyl, and benzyl,
of the formula R⁸—O—CH₂CH(CH₃)—, wherein R⁸ is selected from C3-C14 branched or cyclic aliphatic residues or aromatic residues, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, 2-ethyl-hexyl, C5-C14 cyclic alkyl residues, such as cyclohexyl, and C6-C12 aromatic residues, such as phenyl, 2-methylphenyl, 3-methylphenyl, 4-methylphenyl, and benzyl,
of the formula R⁹—O—CH₂CH(CH₂CH₃)—, wherein R⁹ is selected from C3-C13 branched or cyclic aliphatic residues or aromatic residues, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, 2-ethyl-hexyl, C5-C13 cyclic alkyl residues, such as cyclohexyl, and C6-C12 aromatic residues, such as phenyl, 2-methylphenyl, 3-methylphenyl, 4-methylphenyl, and benzyl,
of the formula R¹⁰—O—CH₂CH₂CH(CH₃)—, wherein R¹⁰ is selected from C3-C13 branched or aromatic residues, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, 2-ethyl-hexyl, C5-C17 cyclic alkyl residues, such as cyclohexyl, and C6-C12 aromatic residues, such as phenyl, 2-methylphenyl, 3-methylphenyl, 4-methylphenyl, and benzyl,
two —O— ether moieties containing alkyl groups, which may be selected from the group consisting of linear, branched, cyclic and aromatic C5-C17 alkyl groups, in particular selected from
two —O— ether moieties containing linear C5-C17 residues of the formula R¹¹—O—CH₂CH₂—O—CH₂CH₂—, wherein R¹¹ is selected from linear C1-C13 alkyl groups, preferably C1-C12 alkyl groups, such as methyl, ethyl, n-propyl, n-butyl, n-pentyl, n-hexyl, n-heptyl or n-octyl, n-nonyl, n-decyl, n-undecyl, and n-dodecyl,
two —O— ether moieties containing branched C7-C17 residues of the formula R¹²—O—CH₂CH(CH₃)—O—CH₂CH(CH₃)—, wherein R¹² is selected from linear C1-C11 alkyl groups, such as methyl, ethyl, n-propyl, n-butyl, n-pentyl, n-hexyl, n-heptyl or n-octyl, n-nonyl, n-decyl or n-undecyl,
two —O— ether moieties containing branched C9-C17 residues of the formula R¹³—O—CH₂CH(CH₂CH₃)—O—CH₂CH(CH₂CH₃)—, wherein R¹³ is selected from linear C1-C9 alkyl groups, such as methyl, ethyl, n-propyl, n-butyl, n-pentyl, n-hexyl, n-heptyl or n-octyl,
two —O— ether moieties containing branched, cyclic or aromatic C7-C17 residues of the formula R¹⁴—O—CH₂CH₂—O—CH₂CH₂—, wherein R¹⁴ is selected from C3-C13 branched or aromatic residues, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, 2-ethyl-hexyl, a C5-C13 cyclic alkyl residue, such as cyclohexyl, and a C6-C12 aromatic residue, such as phenyl, 2-methylphenyl, 3-methylphenyl, 4-methylphenyl, and benzyl,
two —O— ether moieties containing branched, cyclic or aromatic C9-C17 residues of the formula R¹⁵—O—CH₂CH(CH₃)—O—CH₂CH(CH₃)—, wherein R¹⁵ is selected from C3-C11 branched or aromatic residues, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 2-ethyl-hexyl, a C5-C11 cyclic alkyl residue, such as cyclohexyl, and a C6-C11 aromatic residue, such as phenyl, 2-methylphenyl, 3-methylphenyl, 4-methylphenyl, and benzyl,
two —O— ether moieties containing branched, cyclic or aromatic C11-C17 residues of the formula R¹⁶—O—CH₂CH(CH₂CH₃)—O—CH₂CH(CH₂CH₃)—, wherein R¹⁶ is selected from C3-C9 branched or aromatic residues, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 2-ethyl-hexyl, a C5-C9 cyclic alkyl residue, such as cyclohexyl, and a C6-C9 aromatic residue, such as phenyl, 2-methylphenyl, 3-methylphenyl, 4-methylphenyl, and benzyl,
two —O— ether moieties containing branched, cyclic or aromatic C5-C17 residues of the formula R¹⁷—O—CH₂CH(—OR¹³)CH₂—, wherein R¹⁷ and R¹⁸ are independently selected from linear C1-C13 residues, such as methyl, ethyl, n-propyl, n-butyl, n-pentyl, n-hexyl, n-heptyl or n-octyl, n-nonyl, n-decyl, n-undecyl, n-dodecyl groups, branched C3-C16 alkyl groups, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, 2-ethyl-hexyl, cyclic C5-C13 alkyl groups, such as cyclohexyl and aromatic C6-C12 groups, such as phenyl, 2-methylphenyl, 3-methylphenyl, 4-methylphenyl, and benzyl.

In a preferred embodiment according to the invention, a compound for use in cosmetic compositions of the formula (I)

R¹—O—R² (I)
as defined above is provided, which is selected from the group of mono ether compounds (one ether group (—O—) per molecule),
wherein
   R¹ and R² are different from each other and independently selected from C1-C17 linear, branched, cycloaliphatic or aromatic hydrocarbon residues, which contain 0-8, preferred 0-7, more preferred 0-6, even more preferred 0-5, even further preferred 0-3, or preferred 1-8, more preferred 1-7, even more preferred 1-6, further preferred 1-5, even further preferred 1-3, and specifically preferred 1, 2, 3, 4, 5, 6, 7 CH₃ groups,
   the total number of carbon atoms of said compounds is 10-20, preferred 10-17, more preferred 10-15, or preferred 11-15, even more preferred 11-13, and specifically preferred 11, 12, 13, 14, 15,
   the total number of methyl groups (—CH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 4, 5, 6, 7, 8, 9, 10, 11, 12, or 13.

In a preferred embodiment according to the invention, a compound for use in cosmetic compositions of the formula (I)

R¹—O—R² (I)
as defined above is provided, which is selected from the group of di-ether compounds (two ether groups (—O—) per molecule) of the formula (I),
wherein
   R¹ and R² are different from each other and independently selected from C1-C17 linear, branched, cycloaliphatic or aromatic hydrocarbon residues, which contain 0-8, preferred 0-7, more preferred 0-6, even more preferred 0-5, further preferred 0-3, or preferred 1-8, more preferred 1-7, even more preferred 1-6, further preferred 1-5, even further preferred 1-3, and specifically preferred 1, 2, 3, 4, 5, 6, 7 CH₃ groups,
   the total number of carbon atoms of said compounds is 10-20, preferred 10-17, more preferred 10-15, even more preferred 11-15, even further preferred 11-13, and specifically preferred 11, 12, 13, 14, 15,
   the total number of methyl groups (—CH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 4, 5, 6, 7, 8, 9, 10, 11, 12, or 13.

In a preferred embodiment according to the invention, a compound for use in cosmetic compositions of the formula (I)

R¹—O—R² (I)
as defined above is provided, which is selected from the group of tri-ether compounds (three ether groups (—O—) per molecule) of the formula (I),
wherein
   R¹ and R² are different from each other and independently selected from C1-C17 linear, branched, cycloaliphatic or aromatic hydrocarbon residues, which contain 0-8, preferred 0-7, more preferred 0-6, even more preferred 0-5, further preferred 0-3, or preferred 1-8, more preferred 1-7, even more preferred 1-6, further preferred 1-5, even further preferred 1-3, and specifically preferred 1, 2, 3, 4, 5, 6, 7 CH₃ groups,
   the total number of carbon atoms of said compounds is 10-20, preferred 10-17, more preferred 10-15, even more preferred 11-15, even further preferred 11-13, and specifically preferred 11, 12, 13, 14, 15,
   the total number of methyl groups (—CH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 4, 5, 6, 7, 8, 9, 10, 11, 12, or 13.

In a preferred embodiment according to the invention,
the compound for use in cosmetic compositions of the formula (I)

R¹—O—R² (I)
as defined above is a mono-ether compound (one ether group (—O—) per molecule), wherein R¹ and R² are selected as follows:

| R¹ | R² |
|---|---|
| methyl | 3,5,5-trimethyl-hexyl |
| ethyl | 3,5,5-trimethyl-hexyl |
| n-propyl | 3,5,5-trimethyl-hexyl |
| n-butyl | 3,5,5-trimethyl-hexyl, 1,1,3,3-tetramethyl-butyl |
| n-pentyl | 3,5,5-trimethyl-hexyl, 1,1,3,3,5,5-hexamethyl-hexyl, 2,4,4,6,6- |
| | pentamethyl-heptyl, 3,5,5,7,7-pentamethyl-octyl, 1,1,3,3-tetramethyl-butyl |
| n-hexyl | 1,1,3,3-tetramethyl-butyl, 2,4,4-trimethyl-pentyl, 3,5,5-trimethyl-hexyl, 2- |
| | ethyl-hexyl |
| n-heptyl | 1,1,3,3-tetramethyl-butyl, 2,4,4-trimethyl-pentyl, 3,5,5-trimethyl-hexyl, 2- |
| | ethyl-hexyl |
| n-octyl | 3-methyl-pentyl, 3,3-dimethyl-butyl, 2,3-dimethyl-butyl, 1,3-dimethyl-butyl, |
| | 1,1,3,3-tetramethyl-butyl, 2,4,4-trimethyl-pentyl, 3,5,5-trimethyl-hexyl, 2- |
| | ethyl-hexyl |
| n-nonyl | 3-methyl-butyl, 1,1-dimethyl-propyl, 2,2-dimethyl-propyl, 3-methyl-pentyl, |
| | 3,3-dimethyl-butyl, 2,3-dimethyl-butyl, 1,3-dimethyl-butyl, 1,1,3,3- |
| | tetramethyl-butyl, 2,4,4-trimethyl-pentyl, 3,5,5-trimethyl-hexyl, 2-ethyl- |
| | hexyl |
| n-decyl | 2-methyl-propyl, 3-methyl-butyl, t-butyl, 1,1-dimethyl-propyl, 2,2-dimethyl- |
| | propyl, 3-methyl-pentyl, 3,3-dimethyl-butyl, 2,3-dimethyl-butyl, 1,3- |
| | dimethyl-butyl, 1,1,3,3-tetramethyl-butyl, 2,4,4-trimethyl-pentyl, 3,5,5- |
| | trimethyl-hexyl, 2-ethyl-hexyl |
| n-undecyl | iso-propyl, 2-methyl-propyl, 3-methyl-butyl, t-butyl, 1,1-dimethyl-propyl, |
| | 2,2-dimethyl-propyl, 3-methyl-pentyl, 3,3-dimethyl-butyl, 2,3-dimethyl- |
| | butyl, 1,3-dimethyl-butyl, 1,1,3,3-tetramethyl-butyl, 2,4,4-trimethyl-pentyl, |
| | 3,5,5-trimethyl-hexyl, 2-ethyl-hexyl |
| n-dodecyl | iso-propyl, 2-methyl-propyl, 3-methyl-butyl, t-butyl, 1,1-dimethyl-propyl, |
| | 2,2-dimethyl-propyl, 3-methyl-pentyl, 3,3-dimethyl-butyl, 2,3-dimethyl- |
| | butyl, 1,3-dimethyl-butyl, 1,1,3,3-tetramethyl-butyl, 2,4,4-trimethyl-pentyl, |
| | 2-ethyl-hexyl |
| iso-propyl | 3,5,5-trimethyl-hexyl, 2-methylphenyl, 3-methylphenyl, 4-methylphenyl, |
| | benzyl |
| 2-methyl- | 3,5,5-trimethyl-hexyl, cyclohexyl, phenyl, 2-methylphenyl, 3-methylphenyl, |
| propyl | 4-methylphenyl, benzyl |
| 3-methyl-butyl | 3,5,5-trimethyl-hexyl, cyclopentyl, cyclohexyl, phenyl, 2-methylphenyl, 3- |
| | methylphenyl, 4-methylphenyl, benzyl, 1,1,3,3-tetramethyl-butyl |
| t-butyl | 3,5,5-trimethyl-hexyl, cyclohexyl, phenyl, 2-methylphenyl, 3-methylphenyl, |
| | 4-methylphenyl, benzyl |
| 1,1-dimethyl- | 3,5,5-trimethyl-hexyl, cyclopentyl, cyclohexyl, phenyl, 2-methylphenyl, 3- |
| propyl | methylphenyl, 4-methylphenyl, benzyl |
| 2,2-dimethyl- | 3,5,5-trimethyl-hexyl, cyclopentyl, cyclohexyl, phenyl, 2-methylphenyl, 3- |
| propyl | methylphenyl, 4-methylphenyl, benzyl |
| 3-methyl- | 1,1,3,3-tetramethyl-butyl, 2,4,4-trimethyl-pentyl, 3,5,5-trimethyl-hexyl, 2- |
| pentyl | ethyl-hexyl, cyclopentyl, cyclohexyl, phenyl, 2-methylphenyl, 3- |
| | methylphenyl, 4-methylphenyl, benzyl |
| 3,3-dimethyl- | 1,1,3,3-tetramethyl-butyl, 2,4,4-trimethyl-pentyl, 3,5,5-trimethyl-hexyl, 2- |
| butyl | ethyl-hexyl, cyclopentyl, cyclohexyl, phenyl, 2-methylphenyl, 3- |
| | methylphenyl, 4-methylphenyl, benzyl |
| 2,3-dimethyl- | 1,1,3,3-tetramethyl-butyl, 2,4,4-trimethyl-pentyl, 3,5,5-trimethyl-hexyl, 2- |
| butyl | ethyl-hexyl, cyclopentyl, cyclohexyl, phenyl, 2-methylphenyl, 3- |
| | methylphenyl, 4-methylphenyl, benzyl |
| 1,3-dimethyl- | 1,1,3,3-tetramethyl-butyl, 2,4,4-trimethyl-pentyl, 3,5,5-trimethyl-hexyl, 2- |
| butyl | ethyl-hexyl, cyclopentyl, cyclohexyl, phenyl, 2-methylphenyl, 3- |
| | methylphenyl, 4-methylphenyl, benzyl |
| 1,1,3,3- | 2,4,4-trimethyl-pentyl, 3,5,5-trimethyl-hexyl, 2-ethyl-hexyl, cyclopentyl, |
| tetramethyl- | cyclohexyl, phenyl, 2-methylphenyl, 3-methylphenyl, 4-methylphenyl, |
| butyl | benzyl, n-pentyl, n-butyl, isoamyl, isobutyl |
| 2,4,4-trimethyl- | 3,5,5-trimethyl-hexyl, 2-ethyl-hexyl, cyclopentyl, cyclohexyl, phenyl, 2- |
| pentyl | methylphenyl, 3-methylphenyl, 4-methylphenyl, benzyl |
| 3,5,5-trimethyl- | 2-ethyl-hexyl, cyclopentyl, cyclohexyl, phenyl, 2-methylphenyl, 3- |
| hexyl | methylphenyl, 4-methylphenyl, benzyl |
| 1,1,3,3,5,5- | cyclopentyl, cyclohexyl, phenyl, 2-methylphenyl, 3-methylphenyl, 4- |
| hexamethyl- | methylphenyl, benzyl |
| hexyl | |
| 2,4,4,6,6- | cyclopentyl, cyclohexyl, phenyl, 2-methylphenyl, 3-methylphenyl, 4- |
| pentamethyl- | methylphenyl, benzyl |
| heptyl | |
| 3,5,5,7,7- | cyclopentyl, cyclohexyl, phenyl, 2-methylphenyl, 3-methylphenyl, 4- |
| pentamethyl- | methylphenyl, benzyl |
| octyl | |

Therein, it is preferred when the monoether compound of the general formula (I) is selected from the group consisting of 1,1,3,3-tetramethyl-butyl n-butyl ether, 1,1,3,3-tetramethyl-butyl n-pentyl ether, 1,1,3,3-tetramethyl-butyl isoamyl ether, 1,1,3,3-tetramethyl-butyl isobutyl ether, 1,1,3,3-tetramethyl-butyl n-hexyl ether, 1,1,3,3-tetramethyl-butyl n-heptyl ether, 1,1,3,3-tetramethyl-butyl n-octyl ether, 1,1-dimethylpropyl n-nonyl ether, t-butyl n-decyl ether, 1,1-dimethyl-propyl n-decyl ether, t-butyl n-undecyl ether, 1,1-dimethyl-propyl n-undecyl ether, t-butyl n-dodecyl ether, 1,1-dimethyl-propyl n-dodecyl ether, 3,5,5-trimethyl-hexyl iso-propyl ether, 3,5,5-trimethyl-hexyl t-butyl ether, 3,5,5-trimethyl-hexyl 1,1-dimethyl propyl ether and 1,1,3,3-tetramethyl-butyl 3,3-dimethyl-butyl ether.

In a preferred embodiment according to the invention,
the compound for use in cosmetic compositions of the formula (I)

R¹—O—R² (I)
as defined above is a di-ether compound, (two ether groups (—O—) per molecule) of the formula (I),
wherein R² is as defined above, and R¹ is selected from the groups of formula (II)

R¹⁹—O—R²⁰— (II),
and
wherein R¹⁹ is selected from C1-C15 linear, branched, cycloaliphatic or an aromatic hydrocarbyl residue, containing 0-8, preferred 0-7, more preferred 0-6, even more preferred 0-5, further preferred 0-3, or preferred 1-8, more preferred 1-7, even more preferred 1-6, further preferred 1-5, even further preferred 1-3, and specifically preferred 1, 2, 3, 4, 5, 6, or 7 CH₃ groups, and
R²⁰ is selected from C2-C16, preferred C2-C12, more preferred C2-C10, even more preferred C2-C8, even further preferred C2-C6, divalent linear, branched, cyclic, aromatic hydrocarbon residues, such as most preferably —(CH₂)₂—, —(CH₂)₃—, —(CH₂)₄—, —(CH₂)₆—, —(CH₂)₈—, —(CH₂)₁₀—, —(CH₂)₁₂—, —CH₂CH(CH₃)—, —CH₂CH(CH₂CH₃)—, —CH₂CH(CH₂CH₂CH₃)—, —CH₂CH(CH₂CH₂CH₂CH₃)—, and —CH₂CH₂CH(CH₃)—,
the total number of carbon atoms of said compounds is 10-20, preferred 10-17, more preferred 10-15, even more preferred 11-15, even further preferred 11-13, and specifically preferred 11, 12, 13, 14, 15,
the total number of ether groups of said compounds is 2,
the total number of methyl groups (—CH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 4, 5, 6, 7, 8, 9, 10, 11, 12, or 13.

In a preferred embodiment according to the invention,
the compound for use in cosmetic compositions of the formula (I)

R¹—O—R² (I)
as defined above is a di-ether compound (two ether groups (—O—) per molecule),
wherein R¹ is selected from the groups of formula (II)

R¹⁹—O—R²⁰— (II),
and wherein
R², R¹⁹ and R²⁰ are selected as follows:

| R¹⁹ | R²⁰ | R² |
|---|---|---|
| ethyl | —(CH₂)₂—, —(CH₂)₃—, —(CH₂)₄—, | ethyl, t-butyl, 1,1-dimethyl-propyl, |
| | —(CH₂)₆—, —(CH₂)₈—, —(CH₂)₁₀—, | 1,1,3,3-tetramethyl-butyl, 1,1,3,3,5,5- |
| | —(CH₂)₁₂—, | hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| n-propyl | —(CH₂)₂—, —(CH₂)₃—, —(CH₂)₄—, | n-propyl, t-butyl, 1,1-dimethyl-propyl, |
| | —(CH₂)₆—, —(CH₂)₈—, —(CH₂)₁₀—, | 1,1,3,3-tetramethyl-butyl, 1,1,3,3,5,5- |
| | —(CH₂)₁₂—, | hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| n-butyl | —(CH₂)₂—, —(CH₂)₃—, —(CH₂)₄—, | n-butyl, t-butyl, 1,1-dimethyl-propyl, |
| | —(CH₂)₆—, —(CH₂)₈—, —(CH₂)₁₀—, | 1,1,3,3-tetramethyl-butyl, 1,1,3,3,5,5- |
| | —(CH₂)₁₂—, | hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| n-hexyl | —(CH₂)₂—, —(CH₂)₃—, —(CH₂)₄—, | n-hexyl, t-butyl, 1,1-dimethyl-propyl, |
| | —(CH₂)₆—, —(CH₂)₈—, —(CH₂)₁₀—, | 1,1,3,3-tetramethyl-butyl, 1,1,3,3,5,5- |
| | —(CH₂)₁₂—, | hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| isopropyl | —(CH₂)₂—, —(CH₂)₃—, —(CH₂)₄—, | isopropyl, t-butyl, 1,1-dimethyl-propyl, |
| | —(CH₂)₆—, —(CH₂)₈—, —(CH₂)₁₀—, | 1,1,3,3-tetramethyl-butyl, 1,1,3,3,5,5- |
| | —(CH₂)₁₂—, | hexamethyl-hexyl |
| | —CH₂CH(CH₃), —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| 2-methyl- | —(CH₂)₂—, —(CH₂)₃—, —(CH₂)₄—, | 2-methyl-propyl, t-butyl, 1,1-dimethyl- |
| propyl | —(CH₂)₆—, —(CH₂)₈—, —(CH₂)₁₀—, | propyl, 1,1,3,3-tetramethyl-butyl, |
| | —(CH₂)₁₂—, | 1,1,3,3,5,5-hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| 3-methyl- | —(CH₂)₂—, —(CH₂)₃—, —(CH₂)₄—, | 3-methyl-butyl, t-butyl, 1,1-dimethyl- |
| butyl | —(CH₂)₆—, —(CH₂)₈—, —(CH₂)₁₀—, | propyl, 1,1,3,3-tetramethyl-butyl, |
| | —(CH₂)₁₂—, | 1,1,3,3,5,5-hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| t-butyl | —(CH₂)₂—, —(CH₂)₃—, —(CH₂)₄—, | t-butyl, 1,1-dimethyl-propyl, 1,1,3,3- |
| | —(CH₂)₆—, —(CH₂)₈—, —(CH₂)₁₀—, | tetramethyl-butyl, 1,1,3,3,5,5- |
| | —(CH₂)₁₂—, | hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| 1,1- | —(CH₂)₂—, —(CH₂)₃—, —(CH₂)₄—, | 1,1-dimethyl-propyl, 1,1,3,3-tetramethyl- |
| dimethyl- | —(CH₂)₆—, —(CH₂)₈—, —(CH₂)₁₀—, | butyl, 1,1,3,3,5,5-hexamethyl-hexyl |
| propyl | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| 1,1,3,3- | —(CH₂)₂—, —(CH₂)₃—, —(CH₂)₄—, | 1,1,3,3-tetramethyl-butyl, |
| tetramethyl- | —CH₂CH(CH₃)—, | |
| butyl | —CH₂CH(CH₂CH₃)—, | |
| | —CH₂CH₂CH(CH₃)—, | |

Therein, it is preferred when the diether compound of the general formula (I) is selected from the group consisting of 2-(2-ethoxyethoxy)-2,4,4-trimethyl-pentane, 2,2,4-trimethyl-4-(2-propoxyethoxy)pentane, 2-(2-butoxyethoxy)-2,4,4-trimethyl-pentane, 2-(2-butoxy-1-methyl-ethoxy)-2,4,4-trimethyl-pentane, 1-(2-tert-butoxypropoxy)butane, 1-(2-tert-butoxyethoxy)butane, 1-(2-tert-butoxyethoxy)hexane, 1-(2-tert-butoxypropoxy)hexane, 2-(2-isopropoxy-1-methyl-ethoxy)-2-methyl-propane, 2-(2-tert-butoxyethoxy)-2-methyl-propane, 2-(3-tert-butoxypropoxy)-2-methyl-propane, 2-(4-tert-butoxybutoxy)-2-methyl-propane, 2-(6-tert-butoxyhexoxy)-2-methyl-propane, 2-(2-tert-butoxypropoxy)-2-methyl-propane, 1,2-di-tert-butoxybutane, 1,2-di-tert-butoxypentane, 1,2-di-tert-butoxyhexane, 1,3-di-tert-butoxybutane, 2-tert-butoxy-1-ethoxy-butane, 2-tert-butoxy-1-propoxy-butane, 2-methyl-2-(1-methyl-2-propoxy-ethoxy)propane, and 2-(2-tert-butoxyethoxy)-2,4,4-trimethyl-pentane.

In a preferred embodiment according to the invention, the compound for use in cosmetic compositions of the formula (I)

R¹—O—R² (I)
as defined above is a di-ether compound (two ether groups (—O—) per molecule),
wherein R¹ is selected from the groups of formula (II)

R¹⁹—O—R²⁰— (II),
and wherein
R² is t-butyl and R¹⁹ and R²⁰ are as defined above.

In another preferred embodiment according to the invention,
the compound for use in cosmetic compositions of the formula (I)

R¹—O—R² (I)
as defined above is a di-ether compound (two ether groups (—O—) per molecule),
wherein R¹ is selected from the groups of formula (II)

R¹⁹—O—R²⁰— (II),
and wherein
R² is t-butyl and R¹⁹ and R²⁰ are selected as follows:

| R¹⁹ | R²⁰ |
|---|---|
| ethyl | —CH₂CH(CH₂CH₃)—, |
| | —CH₂CH(CH₂CH₂CH₃)—, |
| | —CH₂CH(CH₂CH₂CH₂CH₃)— |
| n-propyl | —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)— |
| n-butyl | —(CH₂)₂—, |
| | —CH₂CH(CH₃)—, —CH₂CH(CH₂CH₃)—, |
| | —CH₂CH(CH₂CH₂CH₃)—, |
| | —CH₂CH(CH₂CH₂CH₂CH₃)—, |
| n-hexyl | —(CH₂)₂—, |
| | —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)—, |
| isopropyl | —CH₂CH(CH₃)—, —CH₂CH(CH₂CH₃)—, |
| | —CH₂CH(CH₂CH₂CH₃)—, |
| | —CH₂CH(CH₂CH₂CH₂CH₃)— |
| 2-methyl-propyl | —(CH₂)₂—, |
| | —CH₂CH(CH₃)—, |
| | —CH₂CH(CH₂CH₃)—, —CH₂CH(CH₂CH₂CH₃)—, |
| | —CH₂CH(CH₂CH₂CH₂CH₃)—, |
| | —CH₂CH₂CH(CH₃)—, |
| 3-methyl-butyl | —(CH₂)₂—, |
| | —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)—, |
| t-butyl | —(CH₂)₂—, —(CH₂)₃—, —(CH₂)₄—, |
| | —(CH₂)₆—, —(CH₂)₈—, —(CH₂)₁₀—, |
| | —(CH₂)₁₂—, |
| | —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)—, |
| | —CH₂CH₂CH(CH₃)—, |
| 1,1-dimethyl-propyl | —(CH₂)₂—, —(CH₂)₃—, —(CH₂)₄—, |
| | —(CH₂)₆—, —(CH₂)₈—, —(CH₂)₁₀—, |
| | —CH₂CH(CH₃)—, —CH₂CH(CH₂CH₃)—, |
| | —CH₂CH(CH₂CH₂CH₃)—, |
| | —CH₂CH(CH₂CH₂CH₂CH₃)—, |
| | —CH₂CH₂CH(CH₃)—, |
| 1,1,3,3-tetramethyl- | —(CH₂)₂—, —(CH₂)₃—, —(CH₂)₄—, |
| butyl | —(CH₂)₆—, —(CH₂)₈—, |
| | —CH₂CH(CH₃)—, —CH₂CH(CH₂CH₃)—, |
| | —CH₂CH(CH₂CH₂CH₃)—, |
| | —CH₂CH(CH₂CH₂CH₂CH₃)—, |
| | —CH₂CH₂CH(CH₃)— |

According to this embodiment, it is preferred when R¹⁹ and R²⁰ are selected in such way that the compound for use in cosmetic compositions of the formula (I) is one of the group consisting of 2-methyl-2-(1-methyl-2-propoxy-ethoxy) propane, 1-(2-tert-butoxypropoxy) butane, 1-(2-tert-butoxyethoxy) butane, 1-(2-tert-butoxypropoxy)hexane, 1-(2-tert-butoxyethoxy)hexane, 2-(2-isopropoxy-1-methyl-ethoxy)-2-methyl-propane, 2-(2-tert-butoxyethoxy)-2-methyl-propane, 2-(3-tert-butoxypropoxy)-2-methyl-propane, 1,4-ditert-butoxybutane, 1,6-ditert-butoxyhexane, 2-(2-tert-butoxypropoxy)-2-methyl-propane, 2-(2-tert-butoxyethoxy)-2-methyl-butane, 2-(3-tert-butoxypropoxy)-2-methyl-butane, 2-(4-tert-butoxybutoxy)-2-methyl-butane, 2-(2-tert-butoxyethoxy)-2,4,4-trimethyl-pentane, 2-(3-tert-butoxypropoxy)-2,4,4-trimethyl-pentane and 2-(4-tert-butoxybutoxy)-2,4,4-trimethyl-pentane.

In a further preferred embodiment according to the invention,
the compound for use in cosmetic compositions of the formula (I)

R¹—O—R² (I)
as defined above is a tri-ether compound (three ether groups (—O—) per molecule), wherein
R² is as defined above,
and R¹ is selected from the groups of formula (III)

R²¹—O—CH₂—CH(OR²¹)—CH₂—(III)
wherein
R²¹ are independently selected from C1-C13 linear, branched, cycloaliphatic or aromatic hydrocarbyl residues, containing 0-8, preferred 0-7, more preferred 0-6, even more preferred 0-5, further preferred 0-3, or preferred 1-8, more preferred 1-7, even more preferred 1-6, further preferred 1-5, even further preferred 1-3, and specifically preferred 1, 2, 3, 4, 5, 6, or 7 CH₃ groups,
the total number of carbon atoms of said compounds is 10-20, preferred 10-17, more preferred 10-15, even more preferred 11-15, even further preferred 11-13, and specifically preferred 11, 12, 13, 14, 15,
the total number of ether groups of said compounds is 3,
the total number of methyl groups (—CH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 4, 5, 6, 7, 8, 9, 10, 11, 12, or 13.

According to this embodiment, it is preferred when R²¹ is independently selected from i-propyl and t-butyl, and it is even more preferred when one R²¹ is selected from i-propyl and t-butyl, while R² is selected from t-butyl.

In a further preferred embodiment according to the invention, the compound for use in cosmetic compositions of the formula (I)
R¹—O—R² (I)
as defined above is a tri-ether compound (three ether groups (—O—) per molecule),
wherein R¹ is selected from the groups of formula (III)

R²¹—O—CH₂—CH(OR²¹)—CH₂— (III)
and R²¹ and R² are independently selected from linear, or branched C1-C5 hydrocarbyl residues, preferred from n-propyl, n-butyl, n-pentyl, isopropyl, 2-methyl-propyl, 3-methyl-butyl, t-butyl, and 1,1-dimethyl-propyl,
the total number of carbon atoms of said compounds is 10-18, preferred 10-17, more preferred 10-15, even more preferred 11-15, even further preferred 11-13, and specifically preferred 11, 12, 13, 14, 15,
the total number of ether groups of said compounds is 3,
the total number of methyl groups (—CH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 4, 5, 6, 7, 8, 9, 10, 11, 12, or 13.

According to this embodiment, it is preferred when one or more of the residues R²¹ and R² are independently selected from isopropyl and t-butyl, more preferably two or more of R²¹ and R² are independently selected from isopropyl and t-butyl, even more preferably both R²¹ groups and R² are selected from isopropyl and t-butyl, and most preferably all groups R²¹ and R² are isopropyl or all groups R²¹ and R² are t-butyl.

It is also preferred when one or both groups R²¹ are independently selected from isopropyl or t-butyl, and it is even more preferred when both R²¹ residues are the same residue t-butyl or isopropyl, most preferably both R²¹ groups are t-butyl.

In a further preferred embodiment according to the invention, the compound for use in cosmetic compositions of the formula (I)

R¹—O—R² (I)
as defined above is a tri-ether compound (three ether groups (—O—) per molecule),
R¹ is selected from the groups of formula (IV)

R²¹—O—R²²—O—R²²— (IV)
wherein
R²¹ and R² are independently selected from C1-C15 linear, branched, cycloaliphatic or aromatic hydrocarbyl residues,
containing 0-8, preferred 0-7, more preferred 0-6, even more preferred 0-5, further preferred 0-3, or preferred 1-8, more preferred 1-7, even more preferred 1-6, further preferred 1-5, even further preferred 1-3, and specifically preferred 1, 2, 3, 4, 5, 6, or 7 CH₃ groups, and
R²² are independently selected from C2-C6 divalent linear, branched, or cyclic aliphatic hydrocarbyl residues or aromatic hydrocarbyl residues, such as preferably —(CH₂)₂—, —CH₂CH(CH₃)—, —CH₂CH(CH₂CH₃)—, —CH₂CH(CH₂CH₂CH₃)—, —CH₂CH(CH₂CH₂CH₂CH₃)—, and phenylene, and preferably the R²² groups in formula (IV) are identical,
the total number of carbon atoms of said compounds is 10-18, preferred 10-17, more preferred 10-15, even more preferred 11-15, even further preferred 11-13, and specifically preferred 11, 12, 13, 14, 15, the total number of ether groups of said compounds is 3,
the total number of methyl groups (—CH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 4, 5, 6, 7, 8, 9, 10, 11, 12, or 13.

According to the embodiment, it is preferred when both groups R²² are identical and selected from —(OH₂)₂—, —CH₂CH(CH₃)—, and —CH₂CH(CH₂CH₃)—,
while R²¹ is selected from isopropyl, n-butyl, t-butyl, isobutyl, isopentyl, 2,4,4-trimethylpentane or 3,5,5-trimethylhexyl, and R² is selected from iso-propyl, t-butyl and 1,1,3,3 tetra methyl butyl.

In another preferred embodiment according to the invention,
the compound for use in cosmetic compositions of the formula (I)

R¹—O—R² (I)
as defined above is a tri-ether compound (three ether groups (—O—) per molecule), wherein
R¹ is selected from the groups of formula (IV)

R²¹—O—R²²—O—R²²— (IV),
wherein

| R²¹ | R²² | R² |
|---|---|---|
| ethyl | —(CH₂)₂—, | ethyl, t-butyl, 1,1-dimethyl-propyl, |
| | —CH₂CH(CH₃)—, | 1,1,3,3-tetramethyl-butyl, 1,1,3,3,5,5- |
| | —CH₂CH(CH₂CH₃)—, | hexamethyl-hexyl |
| | —CH₂CH(CH₂CH₂CH₃)—, | |
| | —CH₂CH(CH₂CH₂CH₂CH₃)— | |
| n-propyl | —(CH₂)₂—, | n-propyl, t-butyl, 1,1-dimethyl-propyl, |
| | —CH₂CH(CH₃)—, | 1,1,3,3-tetramethyl-butyl, 1,1,3,3,5,5- |
| | —CH₂CH(CH₂CH₃)—, | hexamethyl-hexyl |
| | —CH₂CH(CH₂CH₂CH₃)—, | |
| | —CH₂CH(CH₂CH₂CH₂CH₃)— | |
| n-butyl | —(CH₂)₂—, | n-butyl, t-butyl, 1,1-dimethyl-propyl, |
| | —CH₂CH(CH₃)—, | 1,1,3,3-tetramethyl-butyl, 1,1,3,3,5,5- |
| | —CH₂CH(CH₂CH₃)—, | hexamethyl-hexyl |
| | —CH₂CH(CH₂CH₂CH₃)—, | |
| | —CH₂CH(CH₂CH₂CH₂CH₃)— | |
| n-hexyl | —(CH₂)₂—, | n-hexyl, t-butyl, 1,1-dimethyl-propyl, |
| | —CH₂CH(CH₃)—, | 1,1,3,3-tetramethyl-butyl |
| | —CH₂CH(CH₂CH₃)—, | |
| | —CH₂CH(CH₂CH₂CH₃)—, | |
| | —CH₂CH(CH₂CH₂CH₂CH₃)— | |
| isopropyl | —(CH₂)₂—, | isopropyl, t-butyl, 1,1-dimethyl-propyl, |
| | —CH₂CH(CH₃)—, | 1,1,3,3-tetramethyl-butyl, 1,1,3,3,5,5- |
| | —CH₂CH(CH₂CH₃)—, | hexamethyl-hexyl |
| | —CH₂CH(CH₂CH₂CH₃)—, | |
| | —CH₂CH(CH₂CH₂CH₂CH₃)— | |
| 2-methyl- | —(CH₂)₂—, | 2-methyl-propyl, t-butyl, 1,1-dimethyl- |
| propyl | —CH₂CH(CH₃)—, | propyl, 1,1,3,3-tetramethyl-butyl, |
| | —CH₂CH(CH₂CH₃)—, | 1,1,3,3,5,5-hexamethyl-hexyl |
| | —CH₂CH(CH₂CH₂CH₃)—, | |
| | —CH₂CH(CH₂CH₂CH₂CH₃)— | |
| 3-methyl- | —(CH₂)₂—, | 3-methyl-butyl, t-butyl, 1,1-dimethyl- |
| butyl | —CH₂CH(CH₃)—, | propyl, 1,1,3,3-tetramethyl-butyl |
| | —CH₂CH(CH₂CH₃)—, | |
| | —CH₂CH(CH₂CH₂CH₃)— | |
| t-butyl | —(CH₂)₂—, | t-butyl, 1,1-dimethyl-propyl, 1,1,3,3- |
| | —CH₂CH(CH₃)—, | tetramethyl-butyl, 1,1,3,3,5,5- |
| | —CH₂CH(CH₂CH₃)—, | hexamethyl-hexyl |
| | —CH₂CH(CH₂CH₂CH₃)—, | |
| | —CH₂CH(CH₂CH₂CH₂CH₃)— | |
| 1,1- | —(CH₂)₂—, | 1,1-dimethyl-propyl, 1,1,3,3-tetramethyl- |
| dimethyl- | —CH₂CH(CH₃)—, | butyl |
| propyl | —CH₂CH(CH₂CH₃)—, | |
| | —CH₂CH(CH₂CH₂CH₃)—, | |
| | —CH₂CH(CH₂CH₂CH₂CH₃)— | |
| 1,1,3,3- | —(CH₂)₂— | 1,1,3,3-tetramethyl-butyl |
| tetramethyl- | | |
| butyl | | |

According to this embodiment, the residues R²² are selected independently.

Preferably, the groups R²² are identical.

According to this embodiment, it is preferred when R²¹, R²² and R² are selected in such way that the compound for use in cosmetic compositions of the formula (I) is one of the group consisting of 2-[2-(2-eth oxyethoxy) eth oxy]-2-methyl-propane, 2-[2-(2-ethoxyethoxy)ethoxy]-2,4,4-trimethyl-pentane, 2-[2-(2-ethoxy-1-methyl-ethoxy)-1-methyl-ethoxy]-2-methyl-propane, 2-[2-(2-methoxy-1-methyl-ethoxy)-1-methyl-ethoxy]-2,4,4-trimethyl-pentane, 2-methyl-2-[2-(2-propoxyethoxy)ethoxy]propane, 2,2,4-trimethyl-4-[2-(2-propoxyethoxy)ethoxy]pentane, 2-methyl-2-[1-methyl-2-(1-methyl-2-propoxy-ethoxy)ethoxy]propane, 2,2,4-trimethyl-4-[1-methyl-2-(1-methyl-2-propoxy-ethoxy)ethoxy]pentane, 1-[2-(2-tert-butoxyethoxy)ethoxy]butane, 1-[2-(2-tert-butoxypropoxy)propoxy]butane, 1-[2-(2-tert-butoxyethoxy)ethoxy]hexane, 2-[2-(2-isopropoxyethoxy)ethoxy]-2-methyl-propane, 2-[2-(2-isopropoxyethoxy)ethoxy]-2,4,4-trimethyl-pentane, 2-[2-(2-isopropoxy-1-methyl-ethoxy)-1-methyl-ethoxy]-2-methyl-propane, 2-[2-(2-isopropoxy-1-methyl-ethoxy)-1-methyl-ethoxy]-2,4,4-trimethyl-pentane, 2-[2-(2-tert-butoxyethoxy)ethoxy]-2-methyl-propane and 2-[2-(2-tert-butoxypropoxy)propoxy]-2-methyl-propane.

In a further preferred embodiment according to the invention (tri-ethers),
the compound for use in cosmetic compositions of the formula (I)

R¹—O—R² (I)
as defined above is a tri-ether compound (three ether groups (—O—) per molecule), wherein
R¹ is selected from the groups of formula (IV)

R²¹—O—R²²—O—R²²— (IV)
wherein R²¹ and R²² are as defined above,
R² is t-butyl,
the total number of carbon atoms of said compounds is 10-18, preferred 10-17, more preferred 10-15, even more preferred 11-15, even further preferred 11-13, and specifically preferred 11, 12, 13, 14, 15, and the total number of ether groups of said compounds is 3.

According to this embodiment, the residues R²¹ and R²² are selected independently. Preferably, the groups R²² are identical.

It is further preferred that the residues R²² are selected from the groups —(CH₂)₂— and —CH₂CH(CH₃)—, and more preferable both residues R²² are the same.

In still a further preferred embodiment according to the invention, the compound for use in cosmetic compositions of the formula (I)

R¹—O—R² (I)
as defined above is a tri-ether compound (three ether groups (—O—) per molecule), wherein
R¹ is selected from the groups of formula (IV)

R²¹—O—R²²—O—R²²— (IV)
wherein
R² is t-butyl,
R²¹ and R²² are defined as follows:

| R²¹ | R²² |
|---|---|
| ethyl | —CH₂CH₂—, —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)— |
| n-propyl | —CH₂CH₂—, —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)— |
| n-butyl | —(CH₂)₂—, |
| | —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)—, |
| n-hexyl | —(CH₂)₂—, |
| | —CH₂CH(CH₃)—, |
| | —CH₂CH(CH₂CH₃)—, |
| | —CH₂CH(CH₂CH₂CH₃)—, |
| isopropyl | —CH₂CH₂—, —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)— |
| 2-methyl- | —(CH₂)₂—, |
| propyl | —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)—, |
| 3-methyl- | —(CH₂)₂—, |
| butyl | —CH₂CH(CH₃)—, |
| | —CH₂CH(CH₂CH₃)—, |
| | —CH₂CH(CH₂CH₂CH₃)—, |
| t-butyl | —(CH₂)₂—, |
| | —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)— |
| 1,1- | —(CH₂)₂—, |
| dimethyl- | —CH₂CH(CH₃)—, |
| propyl | —CH₂CH(CH₂CH₃)—, |
| | —CH₂CH(CH₂CH₂CH₃)— |
| 1,1,3,3- | —(CH₂)₂—, |
| tetramethyl- | —CH₂CH(CH₃)—, |
| butyl | —CH₂CH(CH₂CH₃)— |

According to this embodiment, the residues R²² are selected independently. Preferably, the groups R²² are identical.

Even more preferably, the residues R²² are identical and selected from the groups —CH₂CH₂— and —CH₂CH(CH₃)—.

In still a further preferred embodiment according to the invention, the compound for use in cosmetic compositions of the formula (I)

R¹—O—R² (I)

as defined above is selected from asymmetric mono ether compounds, symmetric and asymmetric diether compounds derived from C2 to C6 diols, and symmetric and asymmetric tri-ether-compounds derived from glycerol.

The term “asymmetric” denotes that the structure of the compound does not contain a mirror plane or an inversion center. In case of the monoether compounds of the formula (I), this means that R¹ and R² are not identical.

The C2 to C6 diols from which the symmetric or asymmetric diether compounds are derived from may be selected from linear, branched, cycloaliphatic alkylene or aromatic diols such as catechol, brenzcatechin and hydroquinone, preferably from linear alkylene diols such as 1,2-ethane diol, 1,3-propane diol, 1,2-butane diol, 1,3-butane diol, 1,4-butane diol, 1,5-pentane diol, 1,6-hexanediol, branched alkylene diols such as 1,2-propane diol, 2-methyl-1,2-propane diol, 2-methyl-1,3-propane diol, 1,2-butane diol, 1,3-butane diol, 2-methyl-1,4-butane diol, or cycloaliphatic alkylene diols such as 1,3-cyclopentyl diol or 1,4-cyclohexyl diol, more preferably from C2-C4 linear alkylene diols and C3-C4 branched alkylene diols, most preferably from 1,2-ethane diol, 1,3-propane diol, 1,4-butane diol, 1,2-propane diol and 1,2-butane diol.

In case the tri-ether compounds of the formula (I) are derived from glycerol, the glycerol may be O-substituted with three identical hydrocarbyl residues, two identical hydrocarbyl residues and one different hydrocarbyl residue, or with three different hydrocarbyl residues. Therein, it is preferred when one or more of the substituents bonded to the O-atoms of the glycerol are selected from isopropyl and tert-butyl residues, more preferably two or more of the residues are selected from isopropyl and t-butyl residues.

Also preferably, all substituents bonded to the O-atoms of the glycerol are selected from linear and branched alkyl residues, and more preferably the linear and branched alkyl residues bonded to the O-atoms of the glycerol consist exclusively of carbon and hydrogen atoms, i.e. they do not contain further ether oxygen atoms.

Most preferably, the substituents bonded to the O-atoms of the glycerol are independently selected from iso-propyl and t-butyl residues, wherein one, two or all substituents may be tert-butyl residues.

In another preferred embodiment according to the invention, the compound for use in cosmetic compositions of the formula (I)

R¹—O—R² (I)

as defined above has a molecular weight in the range of 150 to 350 g/mol.

Having a molecular weight within this range, the compound of the formula (I) displays an optimal volatility for the use in cosmetic compositions according to the invention.

Preferably, the molecular weight of the compound for use in cosmetic compositions of the formula (I) is in the range of 228 to 310, more preferably 244 to 300, wherein the compound most preferably contains 1, 2 or 3 oxygen atoms.

In yet another preferred embodiment according to the invention, the compound for use in cosmetic compositions of the formula (I)

R¹—O—R²(I)

as defined above has a volatility in the range of 0.1 gm⁻²h⁻¹ to 100 gm⁻²h⁻¹.

According to the invention, the volatility of the molecules of the compound of the formula (I) is measured by pouring 2 g of material in a 90 mm diameter glass petri dish which is kept on a hot plate of constant temperature of 37° C. The loss of weight of material from the petri dish is over 4 hours is used to calculate the volatility.

Preferably, the volatility is in the range of 0.5 to 90 gm⁻²h⁻¹, more preferably in the range of 10 to 80 gm⁻²h⁻¹, and most preferably in the range of 30 to 70 gm⁻²h⁻¹.

In yet another preferred embodiment according to the invention, the compound for use in cosmetic compositions of the formula (I)

R¹—O—R² (I)

as defined above is selected from the group consisting of:

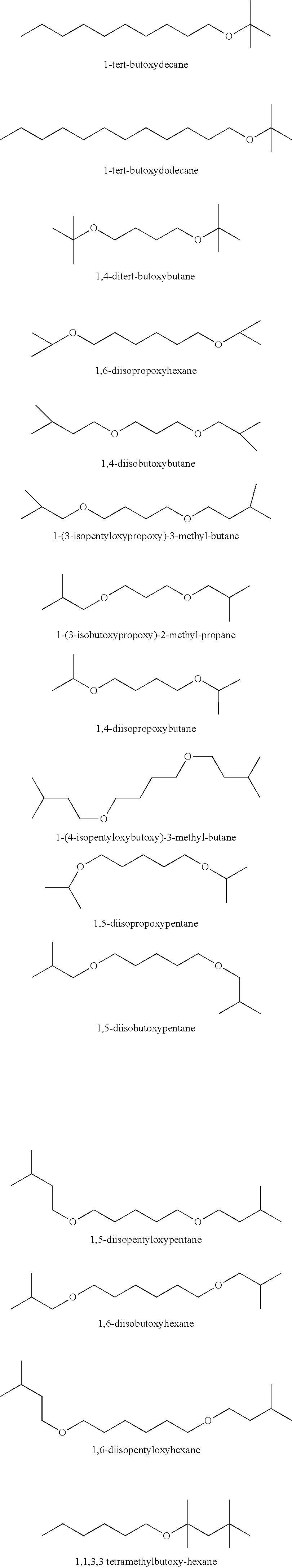

and the further group of mono-ethers consisting of

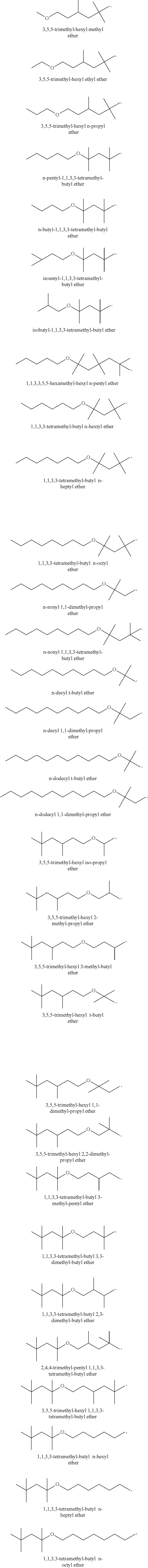

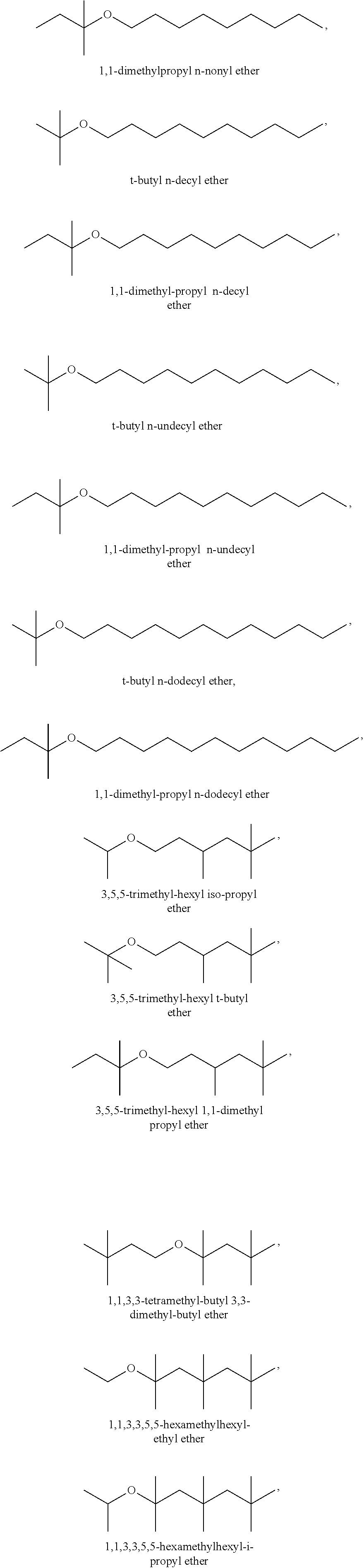

and the further group of di-ethers consisting of

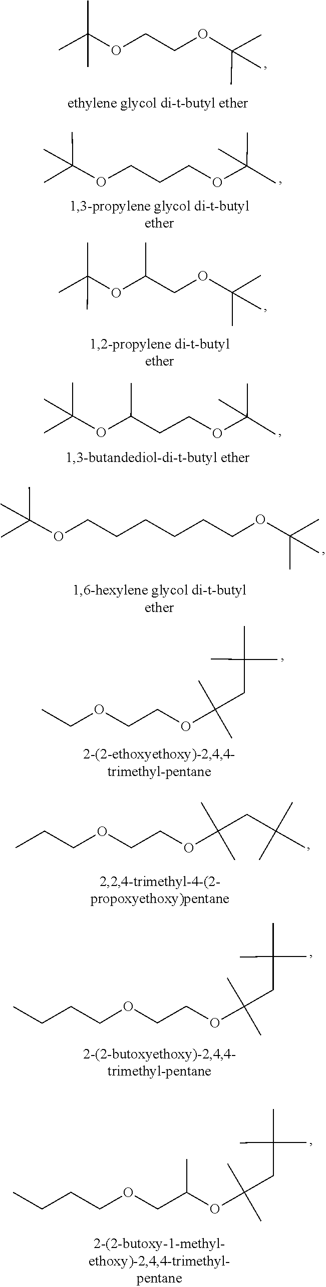

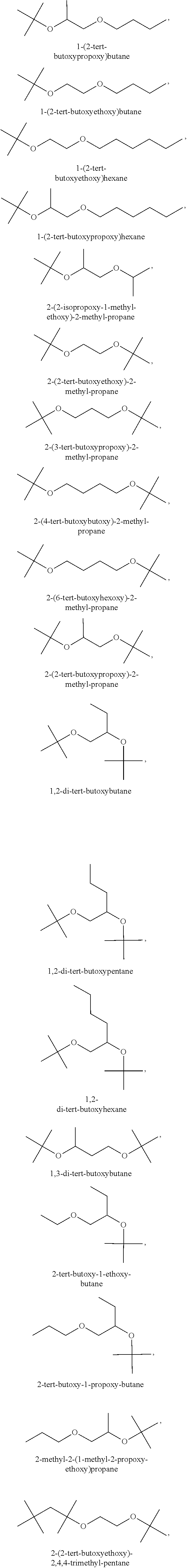

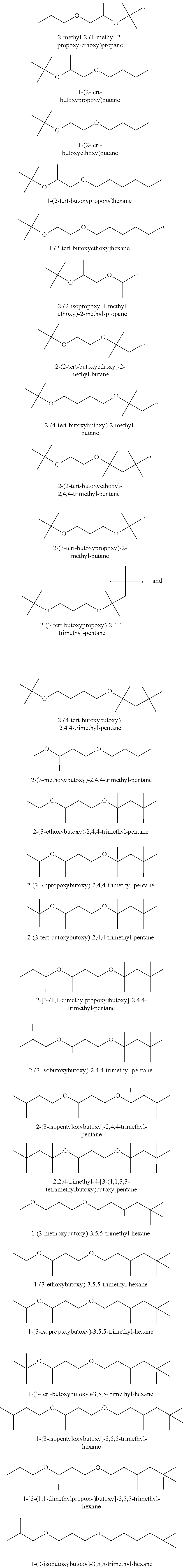

and the further group of tri-ethers consisting of

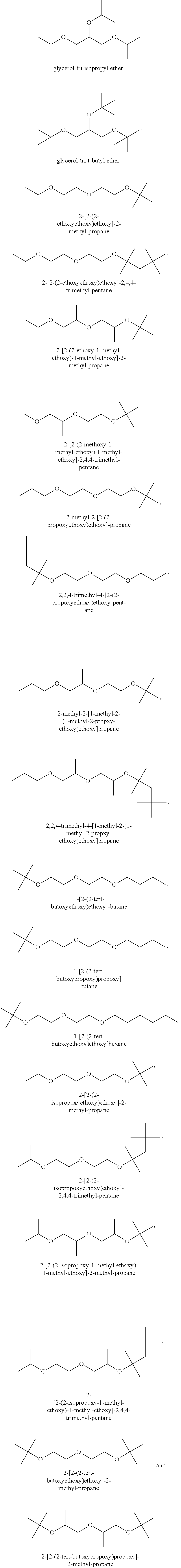

In a preferred embodiment according to the invention, the compound for use in cosmetic compositions of the formula (I)

R¹—O—R² (I)

as defined above is for the preparation of
a. topical compositions to be applied to keratinous substrate such as skin, lip, hair, nail;
b. antiperspirant and deodorant compositions in the form of spray, pencils, sticks, multiphase sticks, pastes, powder, aerosols, creams, cream foams, lotions, self-foaming, foam-like, after-foaming or foamable emulsions, gels, roll-on preparations, foams or depilatories;
c. skin care compositions such as lotions, creams, emulsions and microemulsions, moisturizers, anti-aging products, skin tightening lotions, anti-acne products, day-creams, night-cream, under eye-cream, face mask, face lotion, body lotion, after-shave lotions, cleaning milk, toners
d. color cosmetic compositions such as lip-stick, pencils, lip-color, liquid lip color, lip stain, lip balm, lip-gloss, mascara, eye liner, eye-shadow, foundation, face powder, blush, peel cream, bb cream, cc cream, tinted moisturizer;
e. hair care compositions such as hair treatments, damage repair compositions, hair waxes, hair gels, hair foam, hair styling fluids, hair sprays, hair oil, mousse, shampoo, conditioner, hair remover creams;
f. sun care compositions such as sunscreens, after sun lotions, sun tanner, sunscreen oils, self-tanning products.
g. bath compositions such as bath soap, bath gel, toner, cleansing milk, cleansing solution, make up remover, face wash.

Therein, a topical composition is defined as a composition that is applied directly to a part of the body, in this case to the keratinous substrates such as skin, lip, hair and nail.

A antiperspirant according to the invention is any kind of substance or composition that is put on the skin, especially under the arms, in order to prevent or to reduce sweating. Such antiperspirant according to the invention may be any kind of composition comprising at least one of the volatile compounds according to the invention, however, it is preferably in the form of a spray, stick, multiphase stick, paste, powder, aerosol, cream, cream foam, lotion, self-foaming, foam-like, after-foaming or foamable emulsion, gel, roll-on preparation, foam or depilatories; a deodorant according to the invention is defined as any kind of substance or composition that is applied to the body to prevent or mask body odor due to bacterial breakdown of perspiration in the armpits, groin, and feet, and according to the invention such deodorant may be any kind of composition comprising at least one of the volatile compounds according to the invention, and is preferably in the form of spray, pencils, sticks, multiphase sticks, pastes, powder, aerosols, creams, cream foams, lotions, self-foaming, foam-like, after-foaming or foamable emulsions, gels, roll-on preparations, foams or depilatories. The typical formulations for such products are known to the person skilled in the art.

According to the invention, a skin care composition is defined any composition which may be applied to the skin in order to support skin integrity, enhance its appearance and relieve skin conditions. According to the invention, such skin care composition may be any composition containing at least one of the volatile compounds according to the invention and are typically in the form of lotions, creams, emulsions and microemulsions, moisturizers, anti-aging products, skin tightening lotions, anti-acne products, day-creams, night-cream, under eye-cream, face mask, face lotion, body lotion, after-shave lotions, cleaning milk, toners

According to the invention, a color cosmetic composition denotes any cosmetic composition, i.e. any composition intended to be placed in contact with the various external parts of the human body (epidermis, hair system, nails, lips and external genital organs) or with the teeth and the mucous membranes of the oral cavity with a view exclusively or mainly to cleaning them, perfuming them, changing their appearance and/or correcting body odors and/or protecting them or keeping them in good condition which is primarily directed at the aim of changing the appearance by the provision of color. Typically, these compositions according to the invention containing at least one of the volatile compounds according to the invention are in the form of lip-stick, pencils, lip-color, liquid lip color, lip stain, lip balm, lip-gloss, mascara, eye liner, eye-shadow, foundation, face powder, blush, peel cream, bb cream, cc cream, tinted moisturizer.

According to the invention, a hair care composition is any cosmetic composition applied for care and treatment of hair, in particular on the head.

According to the invention, such hair care composition may be any composition containing at least one of the volatile compounds according to the invention and are typically in the form of hair treatments, damage repair compositions, hair waxes, hair gels, hair foam, hair styling fluids, hair sprays, hair oil, mousse, shampoo, conditioner, hair remover creams.

A sun care composition according to the invention is any kind of skin care, hair care product and nail care product directed at the protection against the effect of sunlight, wherein both the lowering of the amount of radiation interacting with the keratinous parts of the body and alteration of effects of the absorption of radiation, in particular the reduction of skin damage and skin aging, is included. According to the invention, such hair sun care composition may be any such composition containing at least one of the volatile compounds according to the invention and are typically in the form of sunscreens, after sun lotions, sun tanner, sunscreen oils, self-tanning products.

The invention also relates to a cosmetic composition, specifically to a cosmetic composition comprising at least one compound of the formula (I) according to the previous embodiments of the invention, and at least one additional cosmetic ingredient.

Therein, it is preferred when the cosmetic composition contains at least 0.01 weight-% of the compound of the formula (I) based on the total weight of the composition, more preferably 1 to 95 wt-%, even more preferably 2 to 90 weight-%, and most preferably 5 to 80 weight-% based on the total weight of the composition.

According to the invention, a cosmetic ingredient is any compound which may be used in the formulation of products that are used to care for the face and body or to enhance or change the appearance of the face or body.

Therein, oils, waxes, thickeners, humectants, sunscreens, emollients, fats obtained from animals, or minerals, in particular metal oxides, organic compounds acting as colorants, fragrances or preservatives, pigments, natural products and mixtures thereof obtained by extraction of plants, processed plant parts or polymers, emulsifiers and surfactants are preferred.

In a preferred embodiment according to the invention, the cosmetic composition as defined above is an aqueous composition.

According to the invention, a cosmetic composition is considered an aqueous cosmetic composition when it contains at least 10 wt-% of water based on the total weight of the composition. Typically lotions, creams, emulsions are aqueous cosmetic compositions.

It is preferred when the water content of the cosmetic composition as defined above is in the range of 10 to 80 wt-%, more preferably 15 to 70 weight-% of the composition, most preferably 20 to 60 weight-% based on the total weight of the composition.

In a further preferred embodiment according to the invention, the cosmetic composition as defined above comprises from 0.5 to 95 wt-% of the at least one compound according to the previous claims of the formula (I), based on the total weight of the cosmetic composition

When the cosmetic composition is selected from the group of skin care formulation, it is preferred when the composition comprises 0.1 to 90 wt-%, more preferably 2 to 80 wt-%, even more preferably 5 to 70 wt-%, and most preferably 10 to 60 wt % of the at least one compound according to the formula (I) based on the total weight of the cosmetic composition.

When the cosmetic composition is selected from the group of color cosmetics, it is preferred when the composition comprises 1 to 90 wt-%, more preferably 2 to 80 wt-%, even more preferably 3 to 70 wt-%, and most preferably 5 to 60 wt-% of the at least one compound according to the formula (I) based on the total weight of the cosmetic composition.

When the cosmetic composition is selected from the group of hair care formulations, it is preferred when the composition comprises 0.01 to 99 wt-%, more preferably 0.5 to 95 wt-%, even more preferably 1 to 92 wt-%, and most preferably 2 to 90 wt-% of the at least one compound according to the formula (I) based on the total weight of the cosmetic composition.

The invention further relates to one or more compounds of the formula (I′)

R¹—O—R²(I′)
wherein
R¹ and R² are different from each other and are independently selected from the group of C1-C17 hydrocarbyl residues, said hydrocarbyl residues contain up to 8 CH₃ groups and up to 3 ether groups (—O—),
and wherein
the total number of carbon atoms of said compounds is 10 to 20,
the total number of ether groups of said compounds is 1 to 6, and
the total number of methyl groups (—CH₃) of said compounds is 1 to 13,
at least one of R¹ and R² is a branched C3-C17 hydrocarbyl residue,
with the proviso for all monoether compounds containing a total number of one ether group and containing a total number of 10 to 13 carbon atoms that the compounds are selected from the group consisting of 3,5,5-trimethyl-hexyl methyl ether, 3,5,5-trimethyl-hexyl ethyl ether, 3,5,5-trimethyl-hexyl n-propyl ether, 3,5,5-trimethyl-hexyl isopropyl ether, 3,5,5-trimethyl-hexyl 2-butyl ether, 3,5,5-trimethyl-hexyl isobutyl ether and 3,5,5-trimethyl-hexyl tert-butyl ether, n-pentyl-1,1,3,3-tetramethyl-butyl ether, n-butyl-1,1,3,3-tetramethyl-butyl ether, isoamyl-1,1,3,3-tetramethyl-butyl ether, isobutyl-1,1,3,3-tetramethyl-butyl ether,
and with the proviso for all monoether compounds containing a total number of one ether group and a total number of 14 to 20 carbon atoms that
either
both R¹ and R² are independently selected from the group of C5-C9 hydrocarbyl residues, or
R¹ is selected from C5-C17 linear hydrocarbyl residues and R² is selected from the group of branched C3-C9 hydrocarbyl residues, or
one of the groups R¹ and R² is selected from linear C14-C17 hydrocarbyl residues,
and with the exception of all monoether compounds containing at least one of R¹ and R² representing a 2-propyl-heptyl group,
and wherein the one or more compounds are selected from
the monoethers 1-tert-butoxydecane and 1-tert-butoxydodecane,
monoethers derived from di-isobutene, wherein R¹ is a 1,1,3,3-tetramethylbutyl group, and R² is selected from linear C7 to C12 alkyl groups or branched C6-C12 alkyl groups,
monoethers derived from 3,5,5-trimethyl hexanol, wherein R¹ is a 3,5,5-trimethylhexyl group, and R² is selected from linear C2-C11 alkyl groups or branched C3-C11 alkyl groups, preferably a tert-butyl group, in particular the compounds of the structures

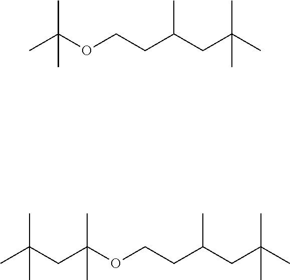

monoethers derived from di-isobutene, wherein R¹ is a 1,1,3,3-tetramethyl-butyl group, and R² is selected from n-butyl, n-pentyl, isobutyl and isoamyl groups,
monoethers derived from tri-isobutene, wherein R¹ is a 1,1,3,3,5,5-hexamethylhexyl group, and R² is selected from linear C2 to C8 alkyl groups or branched C3-C8 alkyl groups, in particular the compounds of the structures

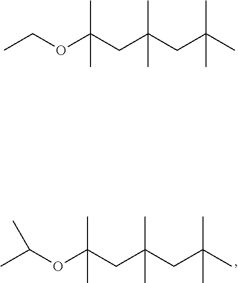

asymmetric diethers derived from ethylene oxide or propylene oxide, i.e. diethers containing ethylene or propylene groups as linker groups between the compounds' two ether —O— atoms, in particular the compounds of the structures

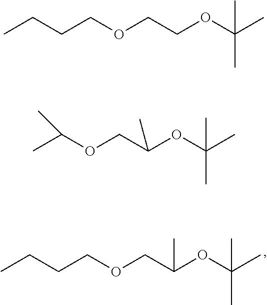

diethers derived from 1,3-butanediol, i.e. diethers containing 1,3-butylene groups as linker groups between the compounds' two ether —O— atoms, in particular the compound of the structure

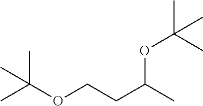

asymmetric diethers derived from 1,3-butanediol, i.e. diethers containing 1,3-butylene groups as linker groups between the compounds' two ether —O— atoms, wherein R¹ is a 1,1,3,3-tetramethylbutyl group, in particular the compounds of the structures

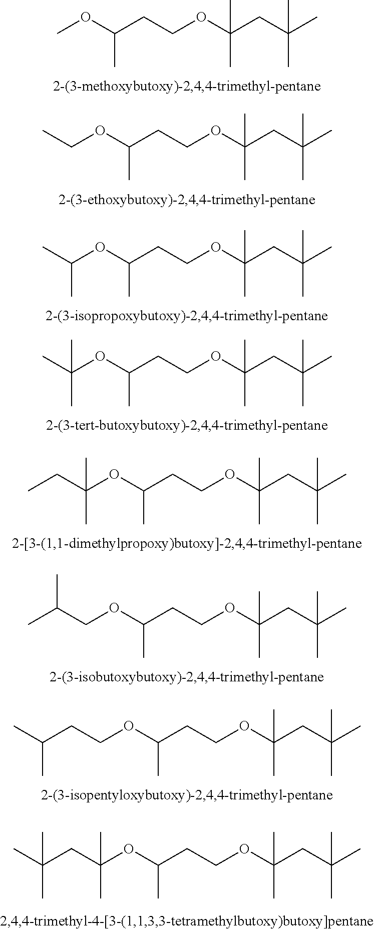

asymmetric diethers derived from 1,3-butanediol, i.e. diethers containing 1,3-butylene groups as linker groups between the compounds' two ether —O— atoms, wherein R¹ is a 3,5,5-trimethylhexyl group, in particular of the structures

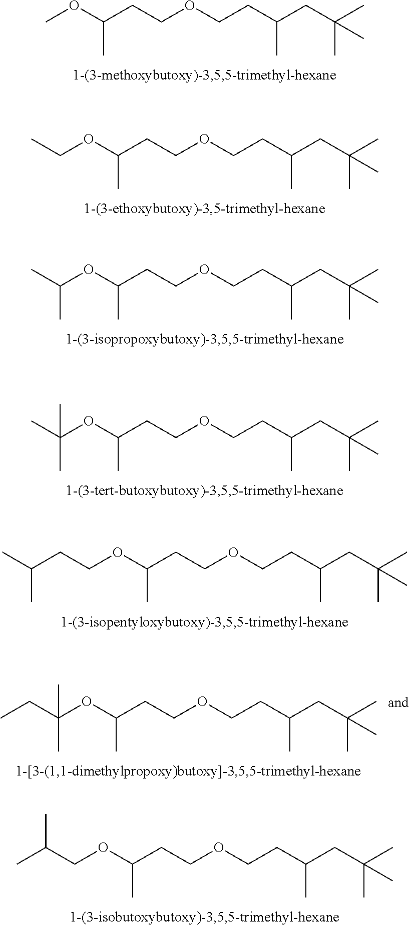

asymmetric triethers derived from ethylene oxide or propylene oxide, i.e. triethers exclusively containing alkylene groups selected from the group consisting of ethylene or propylene groups as linker groups between the compounds' three ether —O— atoms, more preferably containing either two ethylene groups or two propylene groups, in particular the compounds of the structures

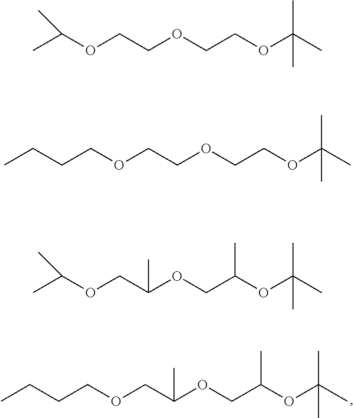

further preferably asymmetric triethers containing either two ethylene groups or two propylene groups as linker groups between the compounds' three ether —O— atoms, wherein one of the two terminal alkyl groups of the compound is a 1,1,3,3-tetramethylbutyl group or a 3,5,5-trimethylhexyl group.

In particular, the invention relates to the following compounds of the formula (I′):

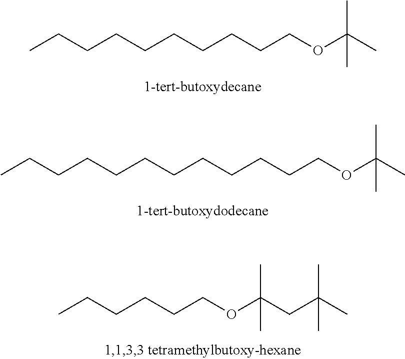

and the further group of mono-ethers consisting of 3,5,5-trimethyl-hexyl methyl ether, 3,5,5-trimethyl-hexyl ethyl ether, 3,5,5-trimethyl-hexyl n-propyl ether, 1,1,3,3,5,5-hexamethyl-hexyl n-pentyl ether, n-pentyl-1,1,3,3-tetramethyl-butyl ether, n-butyl-1,1,3,3-tetramethyl-butyl ether, isoamyl-1,1,3,3-tetramethyl-butyl ether, isobutyl-1,1,3,3-tetramethyl-butyl ether, 1,1,3,3-tetramethyl-butyl n-hexyl ether, 1,1,3,3-tetramethyl-butyl n-heptyl ether, 1,1,3,3-tetramethyl-butyl n-octyl ether, n-nonyl 1,1-dimethyl-propyl ether, n-nonyl 1,1,3,3-tetramethyl-butyl ether, n-decyl t-butyl ether, n-decyl 1,1-dimethyl-propyl ether, n-dodecyl t-butyl ether, n-dodecyl 1,1-dimethyl-propyl ether, 3,5,5-trimethyl-hexyl iso-propyl ether, 3,5,5-trimethyl-hexyl 2-methyl-propyl ether, 3,5,5-trimethyl-hexyl 3-methyl-butyl ether, 3,5,5-trimethyl-hexyl t-butyl ether, 3,5,5-trimethyl-hexyl 1,1-dimethyl-propyl ether, 3,5,5-trimethyl-hexyl 2,2-dimethyl-propyl ether, 1,1,3,3-tetramethyl-butyl 3-methyl-pentyl ether, 1,1,3,3-tetramethyl-butyl 3,3-dimethyl-butyl ether, 1,1,3,3-tetramethyl-butyl 2,3-dimethyl-butyl ether, 2,4,4-trimethyl-pentyl 1,1,3,3-tetramethyl-butyl ether, 3,5,5-trimethyl-hexyl 1,1,3,3-tetramethyl-butyl ether, 1,1,3,3-tetramethyl-butyl n-hexyl ether, 1,1,3,3-tetramethyl-butyl n-heptyl ether, 1,1,3,3-tetramethyl-butyl n-octyl ether, 1,1-dimethylpropyl n-nonyl ether, t-butyl n-decyl ether, 1,1-dimethyl-propyl n-decyl ether, t-butyl n-undecyl ether, 1,1-dimethyl-propyl n-undecyl ether, t-butyl n-dodecyl ether, 1,1-dimethyl-propyl n-dodecyl ether, 3,5,5-trimethyl-hexyl iso-propyl ether, 3,5,5-trimethyl-hexyl t-butyl ether, 3,5,5-trimethyl-hexyl 1,1-dimethyl propyl ether and 1,1,3,3-tetramethyl-butyl 3,3-dimethyl-butyl ether, 1,1,3,3,5,5-hexamethylhexyl-ethyl ether, 1,1,3,3,5,5-hexamethylhexyl-1-propyl ether
the further group of di-ethers consisting of ethylene glycol di-t-butyl ether, 1,3-propylene glycol di-t-butyl ether, 1,2-propylene di-t-butyl ether, 1,3-butandediol-di-t-butyl ether, 1,6-hexylene glycol di-t-butyl ether, 2-(2-ethoxyethoxy)-2,4,4-trimethyl-pentane, 2,2,4-trimethyl-4-(2-propoxyethoxy)pentane, 2-(2-butoxyethoxy)-2,4,4-trimethyl-pentane, 2-(2-butoxy-1-methyl-ethoxy)-2,4,4-trimethyl-pentane, 1-(2-tert-butoxypropoxy)butane, 1-(2-tert-butoxyethoxy)butane, 1-(2-tert-butoxyethoxy)hexane, 1-(2-tert-butoxypropoxy)hexane, 2-(2-isopropoxy-1-methyl-ethoxy)-2-methyl-propane, 2-(2-tert-butoxyethoxy)-2-methyl-propane, 2-(3-tert-butoxypropoxy)-2-methyl-propane, 2-(4-tert-butoxybutoxy)-2-methyl-propane, 2-(6-tert-butoxyhexoxy)-2-methyl-propane, 2-(2-tert-butoxypropoxy)-2-methyl-propane, 1,2-di-tert-butoxybutane, 1,2-di-tert-butoxypentane, 1,2-di-tert-butoxyhexane, 1,3-di-tert-butoxybutane, 2-tert-butoxy-1-ethoxy-butane, 2-tert-butoxy-1-propoxy-butane, 2-methyl-2-(1-methyl-2-propoxy-ethoxy)propane, and 2-(2-tert-butoxyethoxy)-2,4,4-trimethyl-pentane, 2-methyl-2-(1-methyl-2-propoxy-ethoxy)propane, 1-(2-tert-butoxypropoxy)butane, 1-(2-tert-butoxyethoxy)butane, 1-(2-tert-butoxypropoxy)hexane, 1-(2-tert-butoxyethoxy)hexane, 2-(2-isopropoxy-1-methyl-ethoxy)-2-methyl-propane, 2-(2-tert-butoxyethoxy)-2-methyl-propane, 2-(3-tert-butoxypropoxy)-2-methyl-propane, 1,4-ditert-butoxybutane, 1,6-ditert-butoxyhexane, 2-(2-tert-butoxypropoxy)-2-methyl-propane, 2-(2-tert-butoxyethoxy)-2-methyl-butane, 2-(3-tert-butoxypropoxy)-2-methyl-butane, 2-(4-tert-butoxybutoxy)-2-methyl-butane, 2-(2-tert-butoxyethoxy)-2,4,4-trimethyl-pentane, 2-(3-tert-butoxypropoxy)-2,4,4-trimethyl-pentane and 2-(4-tert-butoxybutoxy)-2,4,4-trimethyl-pentane, 2-(3-methoxybutoxy)-2,4,4-trimethyl-pentane, 2-(3-ethoxybutoxy)-2,4,4-trimethyl-pentane, 2-(3-isopropoxybutoxy)-2,4,4-trimethyl-pentane, 2-(3-tert-butoxybutoxy)-2,4,4-trimethyl-pentane, 2-[3-(1,1-dimethylpropoxy)butoxy]-2,4,4-trimethyl-pentane, 2-(3-isobutoxybutoxy)-2,4,4-trimethyl-pentane, 2-(3-isopentyloxybutoxy)-2,4,4-trimethyl-pentane, 2,2,4-trimethyl-4-[3-(1,1,3,3-tetramethylbutoxy)butoxy]pentane, 1-(3-methoxybutoxy)-3,5,5-trimethyl-hexane, 1-(3-ethoxybutoxy)-3,5,5-trimethyl-hexane, 1-(3-isopropoxybutoxy)-3,5,5-trimethyl-hexane, 1-(3-tert-butoxybutoxy)-3,5,5-trimethyl-hexane, 1-(3-isopentyloxybutoxy)-3,5,5-trimethyl-hexane, 1-[3-(1,1-dimethylpropoxy)butoxy]-3,5,5-trimethyl-hexane, and 1-(3-isobutoxybutoxy)-3,5,5-trimethyl-hexane,
and the further group of tri-ethers consisting of glycerol-tri-isopropyl ether, glycerol-tri-t-butyl ether 2-[2-(2-ethoxyethoxy)ethoxy]-2-methyl-propane, 2-[2-(2-ethoxyethoxy)ethoxy]-2,4,4-trimethyl-pentane, 2-[2-(2-ethoxy-1-methyl-ethoxy)-1-methyl-ethoxy]-2-methyl-propane, 2-[2-(2-methoxy-1-methyl-ethoxy)-1-methyl-ethoxy]-2,4,4-trimethyl-pentane, 2-methyl-2-[2-(2-propoxyethoxy)ethoxy]propane, 2,2,4-trimethyl-4-[2-(2-propoxyethoxy)ethoxy]pentane, 2-methyl-2-[1-methyl-2-(1-methyl-2-propoxy-ethoxy)ethoxy]propane, 2,2,4-trimethyl-4-[1-methyl-2-(1-methyl-2-propoxy-ethoxy)ethoxy]pentane, 1-[2-(2-tert-butoxyethoxy)ethoxy]butane, 1-[2-(2-tert-butoxypropoxy)propoxy]butane, 1-[2-(2-tert-butoxyethoxy)ethoxy]hexane, 2-[2-(2-isopropoxyethoxy)ethoxy]-2-methyl-propane, 2-[2-(2-isopropoxyethoxy)ethoxy]-2,4,4-trimethyl-pentane, 2-[2-(2-isopropoxy-1-methyl-ethoxy)-1-methyl-ethoxy]-2-methyl-propane, 2-[2-(2-isopropoxy-1-methyl-ethoxy)-1-methyl-ethoxy]-2,4,4-trimethyl-pentane, 2-[2-(2-tert-butoxyethoxy)ethoxy]-2-methyl-propane and 2-[2-(2-tert-butoxypropoxy)propoxy]-2-methyl-propane.

It is clarified at this point that all compounds of the formula (I′) also fall under the definition of the formula (I).

According to the invention, the compounds of the formula (I′) as described above are preferred compounds for use in cosmetic compositions of the formula (I) as specified in all previous embodiments according to the invention.

The invention further relates to the use of one or more compounds of the formula (I) as defined in any of the previous embodiments in cosmetic compositions.

According to the invention, it is understood that a compound of the formula (I) as defined in any of the previous embodiments, which includes the selection of the compounds of the formula (I′) as defined above, is present in the formulation of a cosmetic composition as defined above and as exemplified in the previous embodiments, in particular in the formulation of
a. topical compositions to be applied to keratinous substrate such as skin, lip, hair, nail;
b. antiperspirant and deodorant compositions in the form of spray, pencils, sticks, multiphase sticks, pastes, powder, aerosols, creams, cream foams, lotions, self-foaming, foam-like, after-foaming or foamable emulsions, gels, roll-on preparations, foams or depilatories;
c. skin care compositions such as lotions, creams, emulsions and microemulsions, moisturizers, anti-aging products, skin tightening lotions, anti-acne products, day-creams, night-cream, under eye-cream, face mask, face lotion, body lotion, after-shave lotions, cleaning milk, toners;
d. color cosmetic compositions such as lip-stick, pencils, lip-color, liquid lip color, lip stain, lip balm, lip-gloss, mascara, eye liner, eye-shadow, foundation, face powder, blush, peel cream, bb cream, cc cream, tinted moisturizer;
e. hair care compositions such as hair treatments, damage repair compositions, hair waxes, hair gels, hair foam, hair styling fluids, hair sprays, hair oil, mousse, shampoo, conditioner, hair remover creams;
f. sun care compositions such as sunscreens, after sun lotions, sun tanner, sunscreen oils, self-tanning products.
g. bath compositions such as bath soap, bath gel, toner, cleansing milk, cleansing lotion.

Therein, the compounds of the formula (I) can be used as solvents, volatiles, spreading agents, emollients, delivery agents, active solubilizers, detackifiers, compatibilizers or diluent. The compounds are preferably used as volatiles. Further preferably, the compounds are used in cosmetic compositions selected from skin care, sun care, color cosmetics, hair care, bath products and AP deo compositions as outlined above.

The invention also relates to the use of one or more compounds of the formula (I″)

R¹—O—R² (I″)
wherein
R¹ and R² are different from each other and are independently selected from the group of C1-C17 hydrocarbyl residues, said hydrocarbyl residues contain up to 8 CH₃ groups and up to 3 ether groups (—O—), and
wherein
the total number of carbon atoms of said compounds is 14 to 20,
the total number of ether groups of said compounds is 1 to 6, and
the total number of methyl groups (—CH₃) of said compounds is 1 to 13,
at least one of R¹ and R² is a branched C3-C17 hydrocarbyl residue,
with the proviso for all monoether compounds that
either
both R¹ and R² are independently selected from the group of C5-C9 hydrocarbyl residues, or
R¹ is selected from C5-C17 linear hydrocarbyl residues and R² is selected from the group of branched C3-C9 hydrocarbyl residues, or
one of the groups R¹ and R² is selected from linear C14-C17 hydrocarbyl residues,
all monoether compounds containing at least one of R¹ and R² representing a 2-propyl-heptyl group are excepted,
and wherein the compounds are used as solvents, volatiles, spreading agents, emollients, delivery agents, active solubilizers, detackifiers or compatibilizers.

It is noted that all compounds of the formula (I″) also fall under the definition of the compound (I) as described in the previous embodiments according to the invention.

The invention thus relates to the use of all specific subgroups of compounds of the formula (I) and the formula (I′) and specific compounds of the formula (I) and (I′) as defined in the embodiments above which also fall under the definition of the compound of formula (I″).

### Synthetic Methods

The preparation of the ether compounds of the formulas (I) and (I′) according to the invention as outlined above is achieved by synthetic methods generally known in the art for ether synthesis.

The synthesis of ethers by alkylation of alcoholates with alkyl halides (Williamson synthesis) is outlined in Houben-Weyl, Methods of Organic Chemistry, vol. VI/3, 4^{th} edition, oxygen compounds I, Georg Thieme Verlag Stuttgart 1965, 24-32.

The synthesis of asymmetric n-octyl-ethers (iso-butyl, iso-amyl, n-nexyl, n-heptyl) by reaction of Na-octanolate with the corresponding branched or linear alkyl bromides is described in L. W. Davaney, JACS, 1953, 75, 4836-4837.

Asymmetric n-octyl- and n-dodecyl-isopropoxy ethers can be synthesized from K-isopropylate and the corresponding n-octyl and n-dodecyl bromides (Y. N. Polivin, Izvestiya Akad. Nauk SSSR, Seriya Khimicheskaya, 1990, 5, 1167-1169).

Alternatively, asymmetric n-octyl and glyceryl ethers (methyl, ethyl, isopropyl and n-butyl) can be synthesized using the corresponding C1 to C4 trialkyl phosphates (FR3049949).

Asymmetric branched ethers can also be synthesized by reaction of primary alcohols with branched aldehydes and ketones in the presence of a Pd catalyst and H₂. Examples for primary alcohols are n-ocatnol, n-decanol, n-dodecanol and butyl diglycol. Examples for branched aldehydes and ketones are 2-methylpropanal, 2,2-dimethylpropanal and 4-methyl-2-pentanone (Y.Fujii et. al., Bull. Chem. Soc. Jpn., 2005, 78, 456-463).

The synthesis of isopopyl di-ethers of diols using acetone as precursor are described in JP 09-316017.

The acid catalyzed etherification of glycerol with alcohols such as 2-propanol can be conducted in the presence of a water removing solvent (Roze et. al, Mat.Sci.and Appl. Chem., 2013, 28, 67-72).

The etherification of glycerol with alcohols such as n-butanol can also be conducted in the presence of a water permeable membrane (C.Canilla et. al, Chem.Eng.J., 2015, 282, 187-193).

The acid catalyzed etherification of alcohols such as glycerol with propene yielding isopropyl ethers is described in C.Saengarum et. al., Hindawi, The Scientific World Journal, 2017, Article ID 4089036, https://doi.org/i0.1155/2017/4089036.

t-Butyl ethers are accessible by acid catalyzed etherification of t-butanol with primary and secondary alcohols (Roze et. al, Mat.Sci.and Appl. Chem., 2013, 28, 67-72. The use of MgSO₄ (S. W. Wright et. al, Tetrahedr. Lett., 1997, 38, 7345-7348) or molecular sieves increases the yield (N.Mallesha et. al, Tetrahedr. Lett., 2012, 53, 641-645).

A broad spectrum of t-butyl ethers can be synthesized by acid catalyzed etherification of isobutene with primary and secondary alcohols.

SU1142465 describes the synthesis of octyl-t-butyl ether using n-octanol and isobutene.

The synthesis of di-t-butyl ethers of di-primary alcohols is described in CS190755 (ethylene glycol) and JPH 0834753 (1,6-hexylene glycol). Secondary alcohol groups containing alcohols such as propylene glycol can also be converted into t-butyl ethers (S. S. Jayadeocar et. al, Reactive Polymers, 1993, 57-67).

The etherification of glycerol with isobutene is described in (J. A. Melero et. al., Appl. Catalysis A General, August 2008, vol. 346, 44-51) and thus known in the art.

The etherification of linear C1-C4 alcohols with di-isobutene was also described (R. S. Karinen et. al, Ind. Eng. Chem. Res., 2001, 40, 6073-6080; R. S. Karinen et. al., Catalysis Letters, 2001, 76(1), 81-87).

Further details on the synthetic protocols yielding mono-ethers and di-ethers are outlined in the example section.

### SUMMARY OF PREFERRED EMBODIMENTS ACCORDING THE INVENTION

In the following, the preferred embodiments according to the invention are summarized:

### Embodiment 1

A compound for use in cosmetic compositions of the formula (I):

R¹—O—R² (I)
wherein
R¹ and R² are different from each other and are independently selected from the group of C1-C17 hydrocarbyl residues, said hydrocarbyl residues contain up to 8 CH₃ groups and up to 3 ether groups (—O—), and wherein
the total number of carbon atoms of said compound is 10 to 20,
the total number of ether groups of said compound is 1 to 6, and
the total number of methyl groups (—CH₃) of said compound is 1 to 13,
at least one of R¹ and R² is a branched C3-C17 hydrocarbyl residue,
with the proviso for all monoether compounds containing a total number of one ether group and containing a total number of 10 to 13 carbon atoms that the compounds are selected from the group consisting of 3,5,5-trimethyl-hexyl methyl ether, 3,5,5-trimethyl-hexyl ethyl ether, 3,5,5-trimethyl-hexyl n-propyl ether, 3,5,5-trimethyl-hexyl isopropyl ether, 3,5,5-trimethyl-hexyl 2-butyl ether, 3,5,5-trimethyl-hexyl isobutyl ether and 3,5,5-trimethyl-hexyl tert-butyl ether, n-pentyl-1,1,3,3-tetramethyl-butyl ether, n-butyl-1,1,3,3-tetramethyl-butyl ether, isoamyl-1,1,3,3-tetramethyl-butyl ether, isobutyl-1,1,3,3-tetramethyl-butyl ether,
and with the proviso for all monoether compounds containing a total number of one ether group and a total number of 14 to 20 carbon atoms that either
both R¹ and R² are independently selected from the groups of C5-C9 hydrocarbyl residues, or
R¹ is selected from C5-C17 linear hydrocarbyl residues and R² is selected from the group of branched C3-C9 hydrocarbyl residues, or
one of the groups R¹ and R² is selected from linear C14-C17 hydrocarbyl residues, and with the exception of all monoether compounds containing at least one of R¹ and R² representing a 2-propyl-heptyl group.

### Embodiment 2

The compound according to the previous embodiment,
wherein the compound has one ether group (—O—) (mono ether compound), and
   at least one of R¹ and R² has at least 6 carbon atoms, and
   at least one of R¹ and R² is a branched alkyl group.

### Embodiment 3

The compound according to the previous embodiments, wherein it is selected from the group of mono-ether compounds (one ether group (—O—) per molecule), di-ether compounds (two ether groups (—O—) per molecule), and tri-ether compounds (three ether groups (—O—) per molecule).

### Embodiment 4

The compound according to the previous embodiments, wherein at least one of R¹ and R² is a branched hydrocarbyl residue and the other residue is selected from linear, branched, cycloaliphatic or aromatic hydrocarbyl groups.

### Embodiment 5

The compound according to the previous embodiments, wherein the number of methyl groups in R¹ and R² is independently 0-7, preferred 0-6, more preferred 0-5, even more preferred 0-3, or preferred 1-7, more preferred 1-6, even more preferred 1-5, further preferred 1-3, specifically preferred 1, 2, 3, 4, 5, 6, or 7.

### Embodiment 6

The compound according to the previous embodiments, wherein the total number of carbon atoms of said compound is 10-17, preferred 10-15, more preferred 11-15, even more preferred 11-13, and specifically preferred 11, 12, 13, 14, or 15.

### Embodiment 7

The compound according to the previous embodiments, wherein the total number of ether groups of said compound is 1 to 5, preferred 1-4, more preferred 1-3, even more preferred 1-2, and specifically preferred 1, 2, or 3.

### Embodiment 8

The compound according to the previous embodiments, wherein the total number of methyl groups (—CH₃) of said compound is 1-12, preferred 1-10, more preferred 1-9, even more preferred 1-7, further preferred 1-6, even further preferred 1-4, or preferred 2-6, more preferred 2-4, and specifically preferred 1, 2, 3, 4, 5, 6, 7, 8, 9, 10, 11, or 12.

### Embodiment 9

The compound according to the previous embodiments, wherein the residues R¹ and R² in formula (I) are independently selected from
alkyl groups, which are selected from the group consisting of linear, branched, or cyclic alkyl groups and aromatic C1-C17 alkyl groups, in particular selected from:
   linear C1-C17 alkyl groups, such as methyl, ethyl, n-propyl, n-butyl, n-pentyl, n-hexyl, n-heptyl or n-octyl, n-nonyl, n-decyl, n-undecyl, n-dodecyl groups,
   branched C3-C17 alkyl groups, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, and 2-ethyl-hexyl,
   cyclic C5-C17 alkyl groups, such as cyclohexyl,
   arylalkyl C7-C13 groups, such as benzyl,
   aromatic C7-C13 alkyl groups, such as, 2-methylphenyl, 3-methylphenyl, 4-methylphenyl,
aromatic C6-C12 groups, such as phenyl groups
one —O— ether moiety containing alkyl groups, which are selected from the group consisting of linear, branched, cyclic and aromatic C3-C17 alkyl groups, in particular, selected from:
   R³—O—CH₂CH₂—,
   wherein R³ is selected from linear C1-C15 alkyl groups, preferably C1-C12 alkyl groups, such as methyl, ethyl, n-propyl, n-butyl, n-pentyl, n-hexyl, n-heptyl or n-octyl, n-nonyl, n-decyl, n-undecyl, and n-dodecyl,
   R⁴—O—CH₂CH(CH₃)—,
   wherein R⁴ is selected from linear C1-C14 alkyl groups, preferably C1-C12 alkyl groups, such as methyl, ethyl, n-propyl, n-butyl, n-pentyl, n-hexyl, n-heptyl or n-octyl, n-nonyl, n-decyl, n-undecyl, and n-dodecyl
   R⁵—O—CH₂CH(CH₂CH₃)—, wherein R⁵ is selected from linear C1-C13 alkyl groups, preferably C1-C12 alkyl groups, such as methyl, ethyl, n-propyl, n-butyl, n-pentyl, n-hexyl, n-heptyl or n-octyl, n-nonyl, n-decyl, n-undecyl, and n-dodecyl,
   R⁶—O—CH₂CH₂CH(CH₃)—, wherein R⁶ is selected from is selected from linear C1-C13 alkyl groups, preferably C1-C12 alkyl groups, such as methyl, ethyl, n-propyl, n-butyl, n-pentyl, n-hexyl, n-heptyl or n-octyl, n-nonyl, n-decyl, n-undecyl, and n-dodecyl,
   R⁷—O—CH₂CH₂—, wherein R⁷ is selected from C3-C15 branched or aromatic residues, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, 2-ethyl-hexyl, C5-C15 cyclic alkyl residues, such as cyclohexyl, and C6-C12 aromatic residues, such as phenyl, 2-methylphenyl, 3-methylphenyl, 4-methylphenyl, and benzyl,
   R⁸—O—CH₂CH(CH₃)—, wherein R⁸ is selected from C3-C14 branched or aromatic residues, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, 2-ethyl-hexyl, C5-C14 cyclic alkyl residues, such as cyclohexyl, and C6-C12 aromatic residues, such as phenyl, 2-methylphenyl, 3-methylphenyl, 4-methylphenyl, and benzyl,
R⁹—O—CH₂CH(CH₂CH₃)—, wherein R⁹ is selected from C3-C13 branched or aromatic residues, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, 2-ethyl-hexyl, C5-C13 cyclic alkyl residues, such as cyclohexyl, and C6-C12 aromatic residues, such as phenyl, 2-methylphenyl, 3-methylphenyl, 4-methylphenyl, and benzyl,
   R¹⁰—O—CH₂CH₂CH(CH₃)—, wherein R¹⁰ is selected from C3-C13 branched or aromatic residues, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, 2-ethyl-hexyl, C5-C13 cyclic alkyl residues, such as cyclohexyl, and C6-C12 aromatic residues, such as phenyl, 2-methylphenyl, 3-methylphenyl, 4-methylphenyl, and benzyl,
two —O— ether moieties containing alkyl groups, which may be selected from the group consisting of linear, branched, cyclic and aromatic C5-C17 alkyl groups, selected in particular from:
   R¹¹—O—CH₂CH₂—O—CH₂CH₂—, wherein R¹¹ is selected from linear C1-C13 alkyl groups, preferably C1-C12 alkyl groups, such as methyl, ethyl, n-propyl, n-butyl, n-pentyl, n-hexyl, n-heptyl or n-octyl, n-nonyl, n-decyl, n-undecyl, and n-dodecyl
   R¹²—O—CH₂CH(CH₃)—O—CH₂CH(CH₃)—, wherein R¹² is selected from linear C1-C11 alkyl groups, such as methyl, ethyl, n-propyl, n-butyl, n-pentyl, n-hexyl, n-heptyl, n-octyl, n-nonyl, n-decyl or n-undecyl,
   R¹³—O—CH₂CH(CH₂CH₃)—O—CH₂CH(CH₂CH₃)—, wherein R¹³ is selected from linear C1-C9 alkyl groups, such as methyl, ethyl, n-propyl, n-butyl, n-pentyl, n-hexyl, n-heptyl and n-octyl derivatives,
   R¹⁴—O—CH₂CH₂—O—CH₂CH₂—, wherein R¹⁴ is selected from C3-C13 branched or aromatic residues, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, 2-ethyl-hexyl, a C5-C13 cyclic alkyl residue, such as cyclohexyl, and a C6-C12 aromatic residue, such as phenyl, 2-methylphenyl, 3-methylphenyl, 4-methylphenyl, and benzyl,
   R¹⁵—O—CH₂CH(CH₃)—O—CH₂CH(CH₃)—, wherein R¹⁵ is selected from C3-C11 branched or aromatic residues, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 2-ethyl-hexyl, a C5-C11 cyclic alkyl residue, such as cyclohexyl, and a C6-C11 aromatic residue, such as phenyl, 2-methylphenyl, 3-methylphenyl, 4-methylphenyl, and benzyl,
   R¹⁶—O—CH₂CH(CH₂CH₃)—O—CH₂CH(CH₂CH₃)—, wherein R¹⁶ is selected from C3-C9 branched or aromatic residues, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 2-ethyl-hexyl, a C5-C9 cyclic alkyl residue, such as cyclohexyl, and a C6-C9 aromatic residue, such as phenyl, 2-methylphenyl, 3-methylphenyl, 4-methylphenyl, and benzyl,
   R¹⁷—O—CH₂CH(—OR¹⁸)CH₂—, wherein R¹⁷ and R¹⁸ are independently selected from linear C1-C13 residues, such as methyl, ethyl, n-propyl, n-butyl, n-pentyl, n-hexyl, n-heptyl or n-octyl, n-nonyl, n-decyl, n-undecyl, n-dodecyl groups, branched C3-C13 alkyl groups, such as iso-propyl, 2-methyl-propyl, 3-methyl-butyl, t-butyl, 1,1-dimethyl-propyl, 2,2-dimethyl-propyl, 3-methyl-pentyl, 3,3-dimethyl-butyl, 2,3-dimethyl-butyl, 1,3-dimethyl-butyl, 1,1,3,3-tetramethyl-butyl, 2,4,4-trimethyl-pentyl, 3,5,5-trimethyl-hexyl, 1,1,3,3,5,5-hexamethyl-hexyl, 2,4,4,6,6-pentamethyl-heptyl, 3,5,5,7,7-pentamethyl-octyl, 2-ethyl-hexyl, cyclic C5-C13 alkyl groups, such as cyclohexyl, and aromatic C6-C12 groups, such as phenyl, 2-methylphenyl, 3-methylphenyl, 4-methylphenyl, and benzyl.

### Embodiment 10

The compound according to the previous embodiments, which is selected from the group of mono ether compounds (one ether group (—O—) per molecule) of the formula (I),
wherein
   R¹ and R² are different from each other and independently selected from C1-C17 linear, branched cycloaliphatic or aromatic hydrocarbon residues, which contain 0-8, preferred 0-7, more preferred 0-6, even more preferred 0-5, even further preferred 0-3, or preferred 1-8, more preferred 1-7, even more preferred 1-6, further preferred 1-5, even further preferred 1-3, and specifically preferred 1, 2, 3, 4, 5, 6, 7 CH₃ groups,
   the total number of carbon atoms of said compounds is 10-20, preferred 10-17, more preferred 10-15, or preferred 11-15, even more preferred 11-13, and specifically preferred 11, 12, 13, 14, 15,
   the total number of methyl groups (—CH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 4, 5, 6, 7, 8, 9, 10, 11, 12, or 13.

### Embodiment 11

The compound according to the previous embodiments 1 and 3-9, wherein it is selected from the group of di-ether compounds (two ether groups (—O—) per molecule) of the formula (I),
wherein
   R¹ and R² are different from each other and independently selected from C1-C17 linear, branched cycloaliphatic or aromatic hydrocarbon residues which contain 0-8, preferred 0-7, more preferred 0-6, even more preferred 0-5, further preferred 0-3, or preferred 1-8, more preferred 1-7, even more preferred 1-6, further preferred 1-5, even further preferred 1-3, and specifically preferred 1, 2, 3, 4, 5, 6, 7 CH₃ groups,
   the total number of carbon atoms of said compounds is 10-20, preferred 10-17, more preferred 10-15, even more preferred 11-15, even further preferred 11-13, and specifically preferred 11, 12, 13, 14, 15,
   the total number of methyl groups (—CH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 4, 5, 6, 7, 8, 9, 10, 11, 12, or 13.

### Embodiment 12

The compound according to the previous embodiments 1 and 3-9, which is selected from the group of tri-ether compounds (three ether groups (—O—) per molecule) of the formula (I),
wherein
   R¹ and R² are different from each other and independently selected from C1-C17 linear, branched, cycloaliphatic or aromatic hydrocarbon residues which contain 0-8, preferred 0-7, more preferred 0-6, even more preferred 0-5, further preferred 0-3, or preferred 1-8, more preferred 1-7, even more preferred 1-6, further preferred 1-5, even further preferred 1-3, and specifically preferred 1, 2, 3, 4, 5, 6, 7 OH₃ groups,
   the total number of carbon atoms of said compounds is 10-20, preferred 10-17, more preferred 10-15, even more preferred 11-15, even further preferred 11-13, and specifically preferred 11, 12, 13, 14, 15, the total number of methyl groups (—OH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 3, 5, 6, 7, 8, 9, 10, 11, 12, or 13.

### Embodiment 13

The compound according to the previous embodiments 1-10 of the formula (I), which is a mono-ether compound, wherein R¹ and R² are selected as follows:

| R¹ | R² |
|---|---|
| methyl | 3,5,5-trimethyl-hexyl |
| ethyl | 3,5,5-trimethyl-hexyl |
| n-propyl | 3,5,5-trimethyl-hexyl |
| n-butyl | 3,5,5-trimethyl-hexyl, 1,1,3,3-tetramethyl-butyl |
| n-pentyl | 3,5,5-trimethyl-hexyl, 1,1,3,3,5,5-hexamethyl-hexyl, 2,4,4,6,6- |
| | pentamethyl-heptyl, 3,5,5,7,7-pentamethyl-octyl, 1,1,3,3- |
| | tetramethyl-butyl |
| n-hexyl | 1,1,3,3-tetramethyl-butyl, 2,4,4-trimethyl-pentyl, 3,5,5-trimethyl- |
| | hexyl, 2-ethyl-hexyl |
| n-heptyl | 1,1,3,3-tetramethyl-butyl, 2,4,4-trimethyl-pentyl, 3,5,5-trimethyl- |
| | hexyl, 2-ethyl-hexyl |
| n-octyl | 3-methyl-pentyl, 3,3-dimethyl-butyl, 2,3-dimethyl-butyl, 1,3- |
| | dimethyl-butyl, 1,1,3,3-tetramethyl-butyl, 2,4,4-trimethyl-pentyl, |
| | 3,5,5-trimethyl-hexyl, 2-ethyl-hexyl |
| n-nonyl | 3-methyl-butyl, 1,1-dimethyl-propyl, 2,2-dimethyl-propyl, 3- |
| | methyl-pentyl, 3,3-dimethyl-butyl, 2,3-dimethyl-butyl, 1,3- |
| | dimethyl-butyl, 1,1,3,3-tetramethyl-butyl, 2,4,4-trimethyl-pentyl, |
| | 3,5,5-trimethyl-hexyl, 2-ethyl-hexyl |
| n-decyl | 2-methyl-propyl, 3-methyl-butyl, t-butyl, 1,1-dimethyl-propyl, 2,2- |
| | dimethyl-propyl, 3-methyl-pentyl, 3,3-dimethyl-butyl, 2,3- |
| | dimethyl-butyl, 1,3-dimethyl-butyl, 1,1,3,3-tetramethyl-butyl, |
| | 2,4,4-trimethyl-pentyl, 3,5,5-trimethyl-hexyl, 2-ethyl-hexyl |
| n-undecyl | iso-propyl, 2-methyl-propyl, 3-methyl-butyl, t-butyl, 1,1-dimethyl- |
| | propyl, 2,2-dimethyl-propyl, 3-methyl-pentyl, 3,3-dimethyl-butyl, |
| | 2,3-dimethyl-butyl, 1,3-dimethyl-butyl, 1,1,3,3-tetramethyl-butyl, |
| | 2,4,4-trimethyl-pentyl, 3,5,5-trimethyl-hexyl, 2-ethyl-hexyl |
| n-dodecyl | iso-propyl, 2-methyl-propyl, 3-methyl-butyl, t-butyl, 1,1-dimethyl- |
| | propyl, 2,2-dimethyl-propyl, 3-methyl-pentyl, 3,3-dimethyl-butyl, |
| | 2,3-dimethyl-butyl, 1,3-dimethyl-butyl, 1,1,3,3-tetramethyl-butyl, |
| | 2,4,4-trimethyl-pentyl, 2-ethyl-hexyl |
| iso-propyl | 3,5,5-trimethyl-hexyl, 2-methylphenyl, 3-methylphenyl, 4- |
| | methylphenyl, benzyl |
| 2-methyl-propyl | 3,5,5-trimethyl-hexyl, cyclohexyl, phenyl, 2-methylphenyl, 3- |
| | methylphenyl, 4-methylphenyl, benzyl |
| 3-methyl-butyl | 3,5,5-trimethyl-hexyl, cyclopentyl, cyclohexyl, phenyl, 2- |
| | methylphenyl, 3-methylphenyl, 4-methylphenyl, benzyl, 1,1,3,3- |
| | tetramethyl-butyl |
| t-butyl | 3,5,5-trimethyl-hexyl, cyclohexyl, phenyl, 2-methylphenyl, 3- |
| | methylphenyl, 4-methylphenyl, benzyl |
| 1,1-dimethyl- | 3,5,5-trimethyl-hexyl, cyclopentyl, cyclohexyl, phenyl, 2- |
| propyl | methylphenyl, 3-methylphenyl, 4-methylphenyl, benzyl |
| 2,2-dimethyl- | 3,5,5-trimethyl-hexyl, cyclopentyl, cyclohexyl, phenyl, 2- |
| propyl | methylphenyl, 3-methylphenyl, 4-methylphenyl, benzyl |
| 3-methyl-pentyl | 1,1,3,3-tetramethyl-butyl, 2,4,4-trimethyl-pentyl, 3,5,5-trimethyl- |
| | hexyl, 2-ethyl-hexyl, cyclopentyl, cyclohexyl, phenyl, 2- |
| | methylphenyl, 3-methylphenyl, 4-methylphenyl, benzyl |
| 3,3-dimethyl-butyl | 1,1,3,3-tetramethyl-butyl, 2,4,4-trimethyl-pentyl, 3,5,5-trimethyl- |
| | hexyl, 2-ethyl-hexyl, cyclopentyl, cyclohexyl, phenyl, 2- |
| | methylphenyl, 3-methylphenyl, 4-methylphenyl, benzyl |
| 2,3-dimethyl-butyl | 1,1,3,3-tetramethyl-butyl, 2,4,4-trimethyl-pentyl, 3,5,5-trimethyl- |
| | hexyl, 2-ethyl-hexyl, cyclopentyl, cyclohexyl, phenyl, 2- |
| | methylphenyl, 3-methylphenyl, 4-methylphenyl, benzyl |
| 1,3-dimethyl-butyl | 1,1,3,3-tetramethyl-butyl, 2,4,4-trimethyl-pentyl, 3,5,5-trimethyl- |
| | hexyl, 2-ethyl-hexyl, cyclopentyl, cyclohexyl, phenyl, 2- |
| | methylphenyl, 3-methylphenyl, 4-methylphenyl, benzyl |
| 1,1,3,3- | 2,4,4-trimethyl-pentyl, 3,5,5-trimethyl-hexyl, 2-ethyl-hexyl, |
| tetramethyl-butyl | cyclopentyl, cyclohexyl, phenyl, 2-methylphenyl, 3-methylphenyl, |
| | 4-methylphenyl, benzyl, n-pentyl, n-butyl, isoamyl, isobutyl |
| 2,4,4-trimethyl- | 3,5,5-trimethyl-hexyl, 2-ethyl-hexyl, cyclopentyl, cyclohexyl, |
| pentyl | phenyl, 2-methylphenyl, 3-methylphenyl, 4-methylphenyl, benzyl |
| 3,5,5-trimethyl- | 2-ethyl-hexyl, cyclopentyl, cyclohexyl, phenyl, 2-methylphenyl, |
| hexyl | 3-methylphenyl, 4-methylphenyl, benzyl |
| 1,1,3,3,5,5- | cyclopentyl, cyclohexyl, phenyl, 2-methylphenyl, 3-methylphenyl, |
| hexamethyl-hexyl | 4-methylphenyl, benzyl |
| 2,4,4,6,6- | cyclopentyl, cyclohexyl, phenyl, 2-methylphenyl, 3-methylphenyl, |
| pentamethyl- | 4-methylphenyl, benzyl |
| heptyl | |
| 3,5,5,7,7- | cyclopentyl, cyclohexyl, phenyl, 2-methylphenyl, 3-methylphenyl, |
| pentamethyl-octyl | 4-methylphenyl, benzyl |

### Embodiment 14

The compound according to the previous embodiments 1, 3-9 and 11 of the formula (I), which is a di-ether compound,
wherein R² is as defined above, and R¹ is selected from the groups of formula (II)

R¹⁹—O—R²⁰— (II),
and
wherein R¹⁹ is selected from C1-C15 linear, branched, cycloaliphatic or aromatic hydrocarbyl residues,
containing 0-8, preferred 0-7, more preferred 0-6, even more preferred 0-5, further preferred 0-3, or preferred 1-8, more preferred 1-7, even more preferred 1-6, further preferred 1-5, even further preferred 1-3, and specifically preferred 1, 2, 3, 4, 5, 6, or 7 CH₃ groups, and
R²⁰ is selected from C2-C16, preferred C2-C12, more preferred C2-C10, even more preferred C2-C8, even further preferred C2-C6, divalent linear, branched, cyclic, aromatic hydrocarbon residues, such as most preferably —(CH₂)₂—, —(CH₂)₃—, —(CH₂)₄—, —(CH₂)₆—, —(CH₂)₈—, —(CH₂)₁₀—, —(CH₂)₁₂—, —CH₂CH(CH₃)—, —CH₂CH(CH₂CH₃)—, —CH₂CH(CH₂CH₂CH₃)—, —CH₂CH(CH₂CH₂CH₂CH₃)—, and —CH₂CH₂CH(CH₃)—,
the total number of carbon atoms of said compounds is 10-20, preferred 10-17, more preferred 10-15, even more preferred 11-15, even further preferred 11-13, and specifically preferred 11, 12, 13, 14, 15, the total number of ether groups of said compounds is 2,
the total number of methyl groups (—OH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 4, 5, 6, 7, 8, 9, 10, 11, 12, or 13.

### Embodiment 15

The compound according to the previous embodiments 1, 3-9, 11 and 14 of the formula (I), which is a di-ether compound,
wherein R¹ is selected from the groups of formula (II)

R¹⁹—O—R²⁰— (II),
and wherein
R², R¹⁹ and R²⁰ are selected as follows:

| R¹⁹ | R²⁰ | R² |
|---|---|---|
| ethyl | —(CH₂)₂—, —(CH₂)₃—, —(CH₂)₄—, | ethyl, t-butyl, 1,1-dimethyl-propyl, |
| | —(CH₂)₆—, —(CH₂)₈—, —(CH₂)₁₀—, | 1,1,3,3-tetramethyl-butyl, 1,1,3,3,5,5- |
| | —(CH₂)₁₂—, | hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| n-propyl | —(CH₂)₂—, —(CH₂)₃—, —(CH₂)₄—, | n-propyl, t-butyl, 1,1-dimethyl-propyl, |
| | —(CH₂)₆—, —(CH₂)₈—, —(CH₂)₁₀—, | 1,1,3,3-tetramethyl-butyl, 1,1,3,3,5,5- |
| | —(CH₂)₁₂—, | hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| n-butyl | —(CH₂)₂—, —(CH₂)₃—, —(CH₂)₄—, | n-butyl, t-butyl, 1,1-dimethyl-propyl, |
| | —(CH₂)₆—, —(CH₂)₈—, —(CH₂)₁₀—, | 1,1,3,3-tetramethyl-butyl, 1,1,3,3,5,5- |
| | —(CH₂)₁₂—, | hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| n-hexyl | —(CH₂)₂—, —(CH₂)₃—, —(CH₂)₄—, | n-hexyl, t-butyl, 1,1-dimethyl-propyl, |
| | —(CH₂)₆—, —(CH₂)₈—, —(CH₂)₁₀—, | 1,1,3,3-tetramethyl-butyl, 1,1,3,3,5,5- |
| | —(CH₂)₁₂—, | hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| isopropyl | —(CH₂)₂—, —(CH₂)₃—, —(CH₂)₄—, | isopropyl, t-butyl, 1,1-dimethyl-propyl, |
| | —(CH₂)₆—, —(CH₂)₈—, —(CH₂)₁₀—, | 1,1,3,3-tetramethyl-butyl, 1,1,3,3,5,5- |
| | —(CH₂)₁₂—, | hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| 2-methyl- | —(CH₂)₂—, —(CH₂)₃—, —(CH₂)₄—, | 2-methyl-propyl, t-butyl, 1,1-dimethyl- |
| propyl | —(CH₂)₆—, —(CH₂)₈—, —(CH₂)₁₀—, | propyl, 1,1,3,3-tetramethyl-butyl, |
| | —(CH₂)₁₂—, | 1,1,3,3,5,5-hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| 3-methyl- | —(CH₂)₂—, —(CH₂)₃—, —(CH₂)₄—, | 3-methyl-butyl, t-butyl, 1,1-dimethyl- |
| butyl | —(CH₂)₆—, —(CH₂)₈—, —(CH₂)₁₀—, | propyl, 1,1,3,3-tetramethyl-butyl, |
| | —(CH₂)₁₂—, | 1,1,3,3,5,5-hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| t-butyl | —(CH₂)₂—, —(CH₂)₃—, —(CH₂)₄—, | t-butyl, 1,1-dimethyl-propyl, 1,1,3,3- |
| | —(CH₂)₆—, —(CH₂)₈—, —(CH₂)₁₀—, | tetramethyl-butyl 1,1,3,3,5,5- |
| | —(CH₂)₁₂—, | hexamethyl-hexyl |
| | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| 1,1- | —(CH₂)₂—, —(CH₂)₃—, —(CH₂)₄—, | 1,1-dimethyl-propyl, 1,1,3,3-tetramethyl- |
| dimethyl- | —(CH₂)₆—, —(CH₂)₈—, —(CH₂)₁₀—, | butyl, 1,1,3,3,5,5-hexamethyl-hexyl |
| propyl | —CH₂CH(CH₃)—, | |
| | —CH₂CH(CH₂CH₃)—, | |
| | —CH₂CH(CH₂CH₂CH₃)—, | |
| | —CH₂CH(CH₂CH₂CH₂CH₃)—, | |
| | —CH₂CH₂CH(CH₃)—, | |
| 1,1,3,3- | —(CH₂)₂—, —(CH₂)₃—, —(CH₂)₄—, | 1,1,3,3-tetramethyl-butyl, |
| tetramethyl- | —CH₂CH(CH₃)—, | |
| butyl | —CH₂CH(CH₂CH₃)—, | |
| | —CH₂CH₂CH(CH₃)—, | |

### Embodiment 16

The compound according to the previous embodiments 1, 3-9, 11 and 14-15 of the formula (I), which is a di-ether compound,
wherein R¹ is selected from the groups of formula (II)
R¹⁹—O—R²⁰—(II),
and wherein
R² is t-butyl and R¹⁹ and R²⁰ are as defined above.

### Embodiment 17

The compound according to the previous embodiments 1, 3-9, 11 and 14-16 of the formula (I), which is a di-ether compound,
wherein R¹ is selected from the groups of formula (II)

R¹⁹—O—R²⁰— (II),
and wherein
R² is t-butyl and R¹⁹ and R²⁰ are selected as follows:

| R¹⁹ | R²⁰ |
|---|---|
| Ethyl | —CH₂CH(CH₂CH₃)—, |
| | —CH₂CH(CH₂CH₂CH₃)—, |
| | —CH₂CH(CH₂CH₂CH₂CH₃)— |
| n-propyl | —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)— |
| n-butyl | —(CH₂)₂—, |
| | —CH₂CH(CH₃)—, —CH₂CH(CH₂CH₃)—, |
| | —CH₂CH(CH₂CH₂CH₃)—, |
| | —CH₂CH(CH₂CH₂CH₂CH₃)—, |
| n-hexyl | —(CH₂)₂—, |
| | —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)—, |
| Isopropyl | —CH₂CH(CH₃)—, —CH₂CH(CH₂CH₃)—, |
| | —CH₂CH(CH₂CH₂CH₃)—, |
| | —CH₂CH(CH₂CH₂CH₂CH₃)— |
| 2-methyl-propyl | —(CH₂)₂—, |
| | —CH₂CH(CH₃)—, |
| | —CH₂CH(CH₂CH₃)—, —CH₂CH(CH₂CH₂CH₃)—, |
| | —CH₂CH(CH₂CH₂CH₂CH₃)—, |
| | —CH₂CH₂CH(CH₃)—, |
| 3-methyl-butyl | —(CH₂)₂—, |
| | —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)—, |
| t-butyl | —(CH₂)₂—, —(CH₂)₃—, —(CH₂)₄—, |
| | —(CH₂)₆—, —(CH₂)₈—, —(CH₂)₁₀—, |
| | —(CH₂)₁₂—, |
| | —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)—, |
| | —CH₂CH₂CH(CH₃)—, |
| 1,1-dimethyl-propyl | —(CH₂)₂—, —(CH₂)₃—, —(CH₂)₄—, |
| | —(CH₂)₆—, —(CH₂)₈—, —(CH₂)₁₀—, |
| | —CH₂CH(CH₃)—, —CH₂CH(CH₂CH₃)—, |
| | —CH₂CH(CH₂CH₂CH₃)—, |
| | —CH₂CH(CH₂CH₂CH₂CH₃)—, |
| | —CH₂CH₂CH(CH₃)—, |
| 1,1,3,3-tetramethyl- | —(CH₂)₂—, —(CH₂)₃—, —(CH₂)₄—, |
| butyl | —(CH₂)₆—, —(CH₂)₈—, |
| | —CH₂CH(CH₃)—, —CH₂CH(CH₂CH₃)—, |
| | —CH₂CH(CH₂CH₂CH₃)—, |
| | —CH₂CH(CH₂CH₂CH₂CH₃)—, |
| | —CH₂CH₂CH(CH₃)— |

### Embodiment 18

The compound according to the previous embodiments 1, 3-9 and 12 of the formula (I), which is a tri-ether compound, wherein
R² is as defined above, and
R¹ is selected from the groups of formula (III)

R²¹—O—CH₂—CH(OR²¹)—CH₂— (III)
wherein
R²¹ are independently selected from C1-C13 linear, branched, cycloaliphatic or aromatic hydrocarbyl residues, containing 0-8, preferred 0-7, more preferred 0-6, even more preferred 0-5, further preferred 0-3, or preferred 1-8, more preferred 1-7, even more preferred 1-6, further preferred 1-5, even further preferred 1-3, and specifically preferred 1, 2, 3, 4, 5, 6, or 7 CH₃ groups,
the total number of carbon atoms of said compounds is 10-20, preferred 10-17 more preferred 10-15, even more preferred 11-15, even further preferred 11-13, and specifically preferred 11, 12, 13, 14, 15, the total number of ether groups of said compounds is 3,
the total number of methyl groups (—CH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 4, 5, 6, 7, 8, 9, 10, 11, 12, or 13.

### Embodiment 19

The compound according to the previous embodiments 1, 3-9, 12 and 18 of the formula (I), which is a tri-ether compound, wherein
R¹ is selected from the groups of formula (III)

R²¹—O—CH₂—CH(OR²¹)—CH₂— (III)

and R²¹ and R² are independently selected from linear, or branched C1-C5 hydrocarbyl residues, preferred from n-propyl, n-butyl, n-pentyl, isopropyl, 2-methyl-propyl, 3-methyl-butyl, t-butyl, and 1,1-dimethyl-propyl,
the total number of carbon atoms of said compounds is 10-18, preferred 10-17, more preferred 10-15, even more preferred 11-15, even further preferred 11-13, and specifically preferred 11, 12, 13, 14, 15, the total number of ether groups of said compounds is 3,
the total number of methyl groups (—CH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 4, 5, 6, 7, 8, 9, 10, 11, 12, or 13.

### Embodiment 20

The compound according to the previous embodiments 1, 3-9, 12 and 18-19 of the formula (I), which is a tri-ether compound, wherein
R¹ is selected from the groups of formula (IV)

R²¹—O—R²²—O—R²²— (IV)
wherein
R²¹ and R² are independently selected from C1-C15 linear, branched, cyclic, aromatic hydrocarbyl residues,
containing 0-8, preferred 0-7, more preferred 0-6, even more preferred 0-5, further preferred 0-3, or preferred 1-8, more preferred 1-7, even more preferred 1-6, further preferred 1-5, even further preferred 1-3, and specifically preferred 1, 2, 3, 4, 5, 6, or 7 CH₃ groups, and
R²² are independently selected from C2-C6 divalent linear, branched, or cyclic aliphatic hydrocarbyl residues or aromatic hydrocarbyl residues, such as preferably —(CH₂)₂—, —CH₂CH(CH₃)—, —CH₂CH(CH₂CH₃)—, —CH₂CH(CH₂CH₂CH₃)—, —CH₂CH(CH₂CH₂CH₂CH₃)—, and phenylene, and preferably the R²² groups in formula (IV) are identical,
the total number of carbon atoms of said compounds is 10-18, preferred 10-17, more preferred 10-15, even more preferred 11-15, even further preferred 11-13, and specifically preferred 11, 12, 13, 14, 15, the total number of ether groups of said compounds is 3,
the total number of methyl groups (—CH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 4, 5, 6, 7, 8, 9, 10, 11, 12, or 13.

### Embodiment 21

The compound according to the previous embodiments 1, 3-9, 12 and 18-20 of the formula (I), which is a tri-ether compound, wherein
R¹ is selected from the groups of formula (IV)

R²¹—O—R²²—O—R²²— (IV),

wherein

| R²¹ | R²² | R² |
|---|---|---|
| ethyl | —(CH₂)₂—, | ethyl, t-butyl, 1,1-dimethyl-propyl, |
| | —CH₂CH(CH₃)—, | 1,1,3,3-tetramethyl-butyl, 1,1,3,3,5,5- |
| | —CH₂CH(CH₂CH₃)—, | hexamethyl-hexyl |
| | —CH₂CH(CH₂CH₂CH₃)—, | |
| | —CH₂CH(CH₂CH₂CH₂CH₃)— | |
| n-propyl | —(CH₂)₂—, | n-propyl, t-butyl, 1,1-dimethyl-propyl, |
| | —CH₂CH(CH₃)—, | 1,1,3,3-tetramethyl-butyl, 1,1,3,3,5,5- |
| | —CH₂CH(CH₂CH₃)—, | hexamethyl-hexyl |
| | —CH₂CH(CH₂CH₂CH₃)—, | |
| | —CH₂CH(CH₂CH₂CH₂CH₃)— | |
| n-butyl | —(CH₂)₂—, | n-butyl, t-butyl, 1,1-dimethyl-propyl, |
| | —CH₂CH(CH₃)—, | 1,1,3,3-tetramethyl-butyl, 1,1,3,3,5,5- |
| | —CH₂CH(CH₂CH₃)—, | hexamethyl-hexyl |
| | —CH₂CH(CH₂CH₂CH₃)—, | |
| | —CH₂CH(CH₂CH₂CH₂CH₃)— | |
| n-hexyl | —(CH₂)₂—, | n-hexyl, t-butyl, 1,1-dimethyl-propyl, |
| | —CH₂CH(CH₃)—, | 1,1,3,3-tetramethyl-butyl |
| | —CH₂CH(CH₂CH₃)—, | |
| | —CH₂CH(CH₂CH₂CH₃)—, | |
| | —CH₂CH(CH₂CH₂CH₂CH₃)— | |
| isopropyl | —(CH₂)₂—, | isopropyl, t-butyl, 1,1-dimethyl-propyl, |
| | —CH₂CH(CH₃)—, | 1,1,3,3-tetramethyl-butyl, 1,1,3,3,5,5- |
| | —CH₂CH(CH₂CH₃)—, | hexamethyl-hexyl |
| | —CH₂CH(CH₂CH₂CH₃)—, | |
| | —CH₂CH(CH₂CH₂CH₂CH₃)— | |
| 2-methyl- | —(CH₂)₂—, | 2-methyl-propyl, t-butyl, 1,1-dimethyl- |
| propyl | —CH₂CH(CH₃)—, | propyl, 1,1,3,3-tetramethyl-butyl, |
| | —CH₂CH(CH₂CH₃)—, | 1,1,3,3,5,5-hexamethyl-hexyl |
| | —CH₂CH(CH₂CH₂CH₃)—, | |
| | —CH₂CH(CH₂CH₂CH₂CH₃)— | |
| 3-methyl- | —(CH₂)₂—, | 3-methyl-butyl, t-butyl, 1,1-dimethyl- |
| butyl | —CH₂CH(CH₃)—, | propyl, 1,1,3,3-tetramethyl-butyl |
| | —CH₂CH(CH₂CH₃)—, | |
| | —CH₂CH(CH₂CH₂CH₃)— | |
| t-butyl | —(CH₂)₂—, | t-butyl, 1,1-dimethyl-propyl, 1,1,3,3- |
| | —CH₂CH(CH₃)—, | tetramethyl-butyl, 1,1,3,3,5,5- |
| | —CH₂CH(CH₂CH₃)—, | hexamethyl-hexyl |
| | —CH₂CH(CH₂CH₂CH₃)—, | |
| | —CH₂CH(CH₂CH₂CH₂CH₃)— | |
| 1,1- | —(CH₂)₂— | 1,1-dimethyl-propyl, 1,1,3,3-tetramethyl- |
| dimethyl- | —CH₂CH(CH₃)—, | butyl |
| propyl | —CH₂CH(CH₂CH₃)—, | |
| | —CH₂CH(CH₂CH₂CH₃)—, | |
| | —CH₂CH(CH₂CH₂CH₂CH₃)— | |
| 1,1,3,3- | —(CH₂)₂— | 1,1,3,3-tetramethyl-butyl |
| tetramethyl- | | |
| butyl | | |

### Embodiment 22

The compound according to the previous embodiments 1, 3-9, 12 and 18-21 of the formula (I), which is a tri-ether compound, wherein
R¹ is selected from the groups of formula (IV)

R²¹—O—R²²—O—R²²— (IV)
wherein R²¹ and R²² are as defined above
R² is t-butyl,
the total number of carbon atoms of said compounds is 10-18, preferred 10-17, more preferred 10-15, even more preferred 11-15, even further preferred 11-13, and specifically preferred 11, 12, 13, 14, 15, and
the total number of ether groups of said compounds is 3.

### Embodiment 23

The compound according to the previous embodiments 1, 3-9, 12 and 18-22 of the formula (I), which is a tri-ether compound, wherein
R¹ is selected from the groups of formula (IV)

R²¹—O—R²²—O—R²²— (IV)
wherein
R² is t-butyl,
R²¹ and R²² are defined as follows:

| R²¹ | R²² |
|---|---|
| ethyl | —CH₂CH₂—, —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)— |
| n-propyl | —CH₂CH₂—, —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)— |
| n-butyl | —(CH₂)₂—, |
| | —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)—, |
| n-hexyl | —(CH₂)₂—, |
| | —CH₂CH(CH₃)—, |
| | —CH₂CH(CH₂CH₃)—, |
| | —CH₂CH(CH₂CH₂CH₃)—, |
| isopropyl | —CH₂CH₂—, —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)— |
| 2-methyl- | —(CH₂)₂—, |
| propyl | —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)—, |
| 3-methyl- | —(CH₂)₂—, |
| butyl | —CH₂CH(CH₃)—, |
| | —CH₂CH(CH₂CH₃)—, |
| | —CH₂CH(CH₂CH₂CH₃)—, |
| t-butyl | —(CH₂)₂—, |
| | —CH₂CH(CH₃)—, |
| | —CH₂CH(CH₂CH₃)—, |
| | —CH₂CH(CH₂CH₂CH₃)—, |
| | —CH₂CH(CH₂CH₂CH₂CH₃)— |
| 1,1- | —(CH₂)₂—, |
| dimethyl- | —CH₂CH(CH₃)—, |
| propyl | —CH₂CH(CH₂CH₃)—, |
| | —CH₂CH(CH₂CH₂CH₃)— |
| 1,1,3,3- | —(CH₂)₂—, |
| tetramethyl- | —CH₂CH(CH₃)—, |
| butyl | —CH₂CH(CH₂CH₃)— |

### Embodiment 24

The compound according to the previous embodiments of the formula (I), which is selected from asymmetric mono ether compounds, symmetric and asymmetric diether compounds derived from C2 to C6 diols, and symmetric and asymmetric tri-ether-compounds derived from glycerol.

### Embodiment 25

The compound according to the previous embodiments of the formula (I), having a molecular weight in the range of 150 to 350 g/mol.

### Embodiment 26

The compound according to the previous embodiments of the formula (I), having a volatility in the range of 0.1 gm⁻²h⁻¹ to 100 gm⁻²h⁻¹ as measured by pouring 2 g of material in a 90 mm diameter glass petri dish which is kept on a hot plate of constant temperature of 37° C., and calculating the volatility by determination of the loss of weight of material from the petri dish over 2 hours.

### Embodiment 27

A compound according to the previous embodiments of the formula (I), which is selected from the group consisting of:

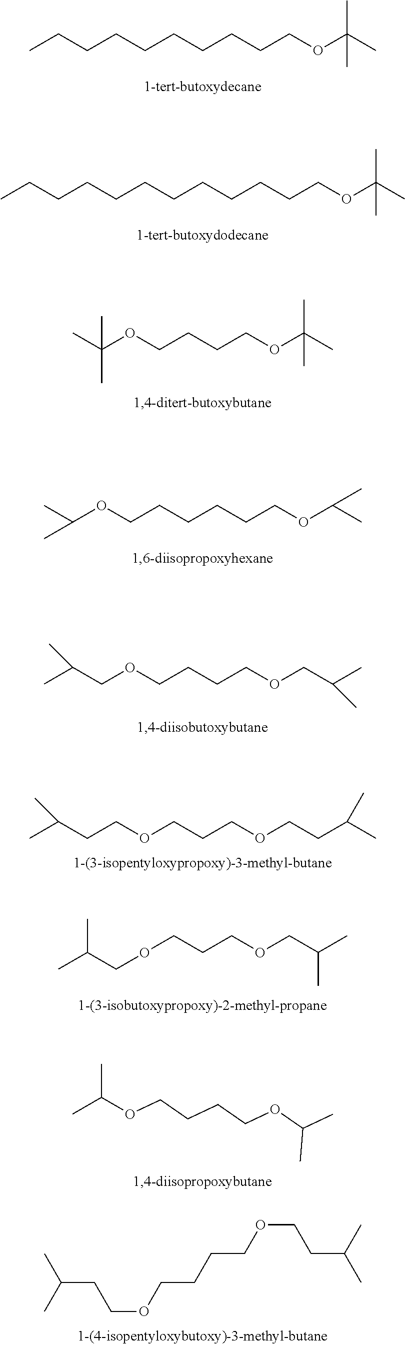

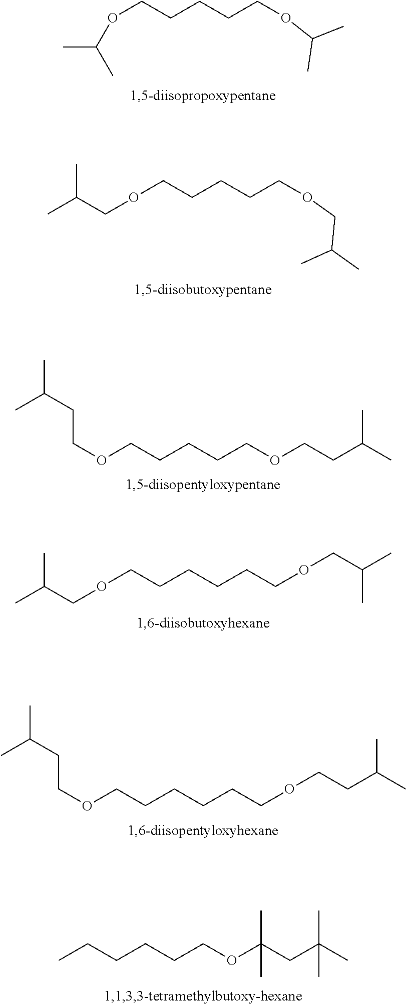

and the further group of mono-ethers consisting of

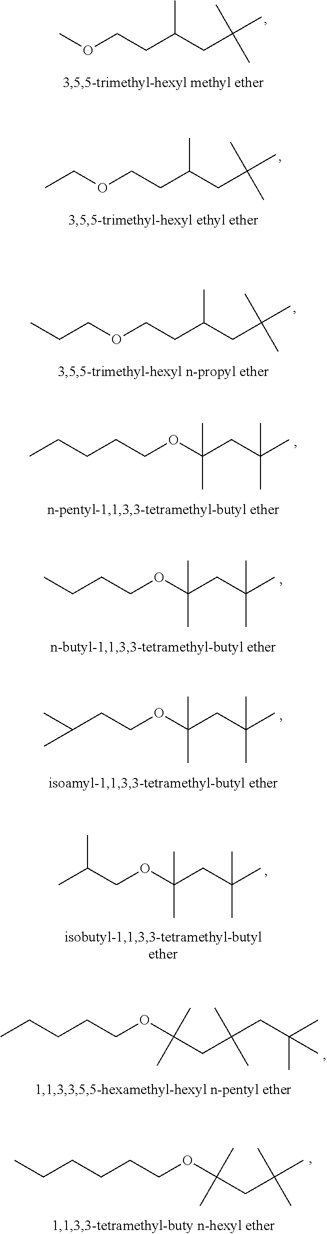

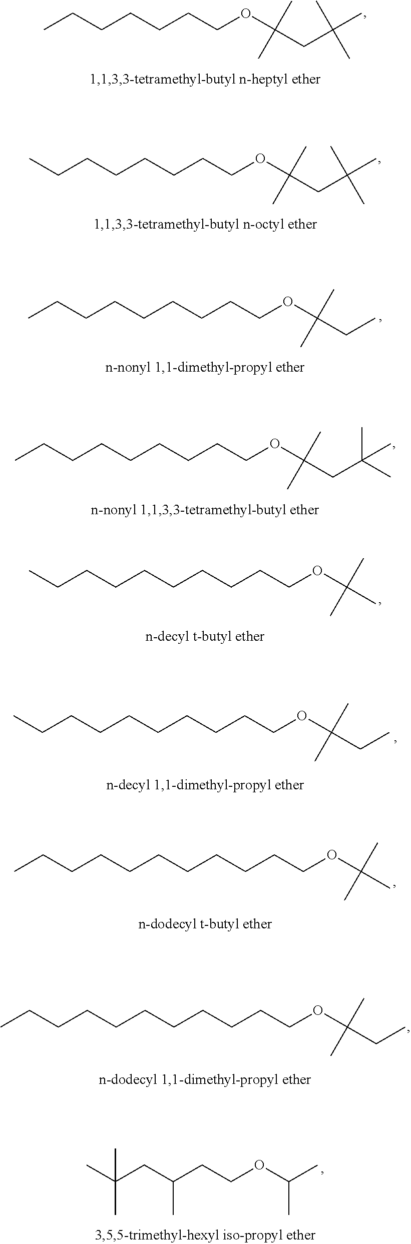

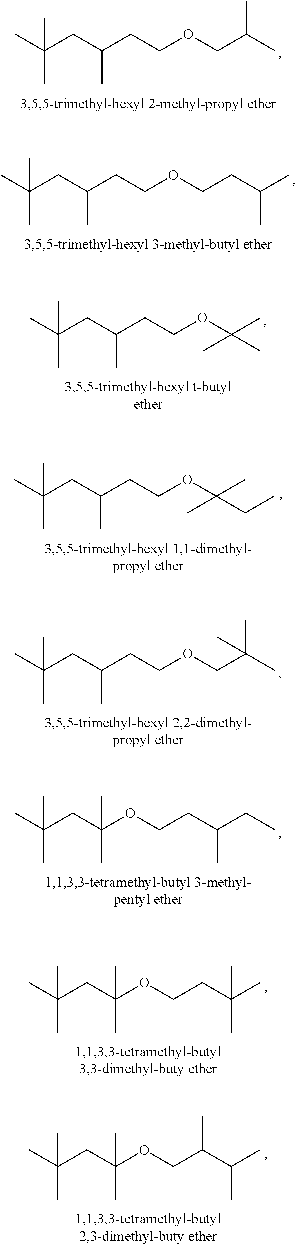

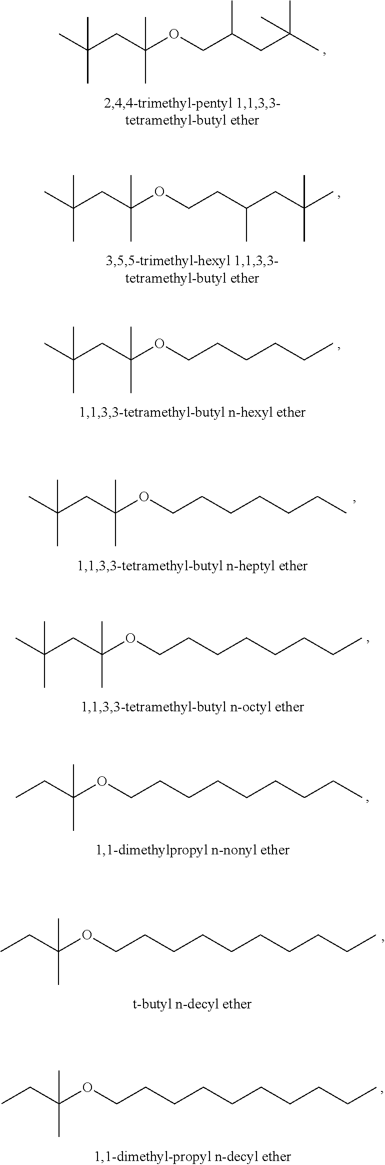

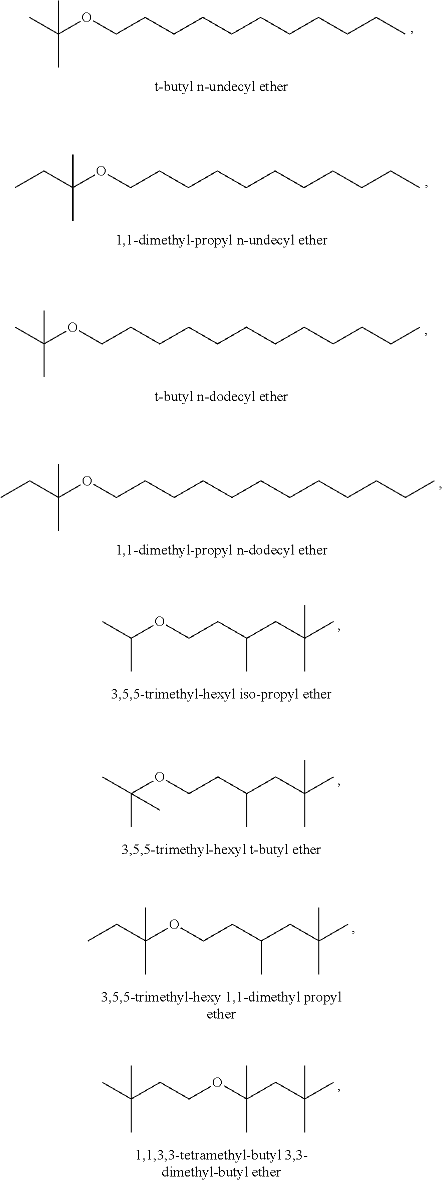

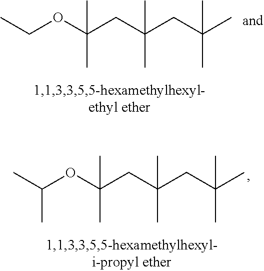

the further group of di-ethers consisting of

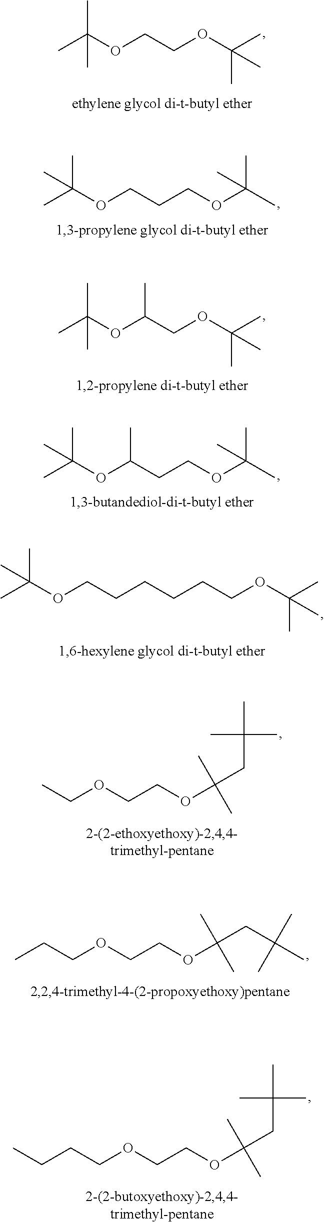

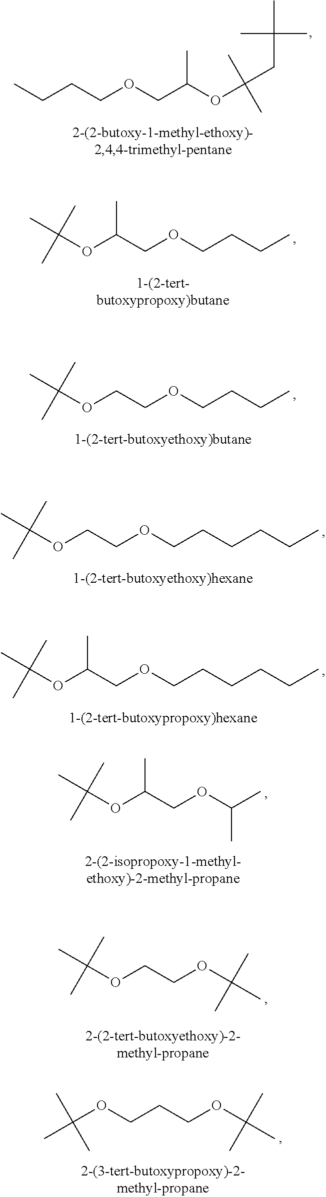

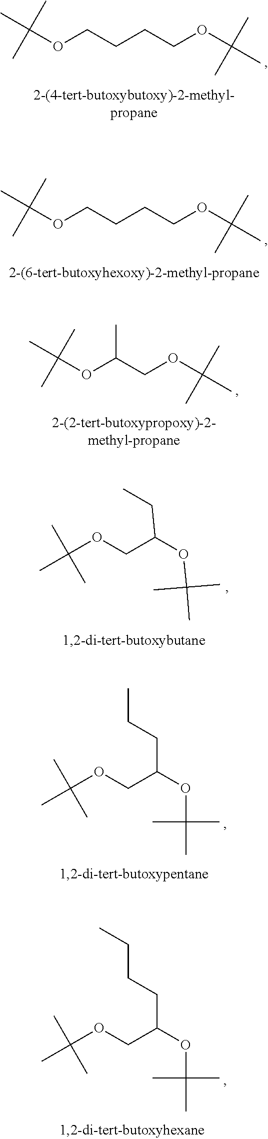

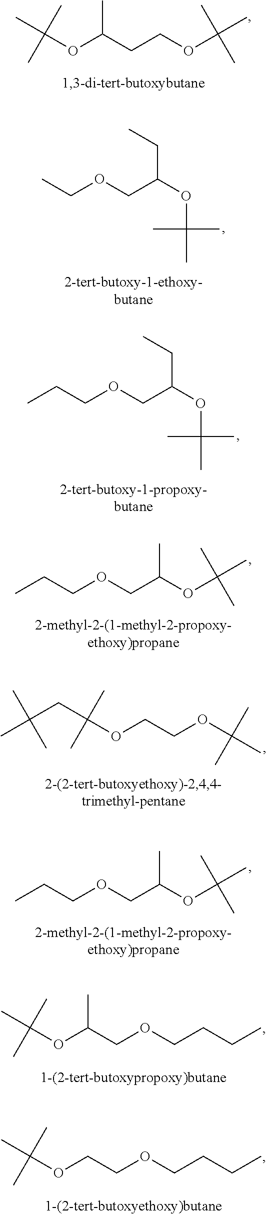

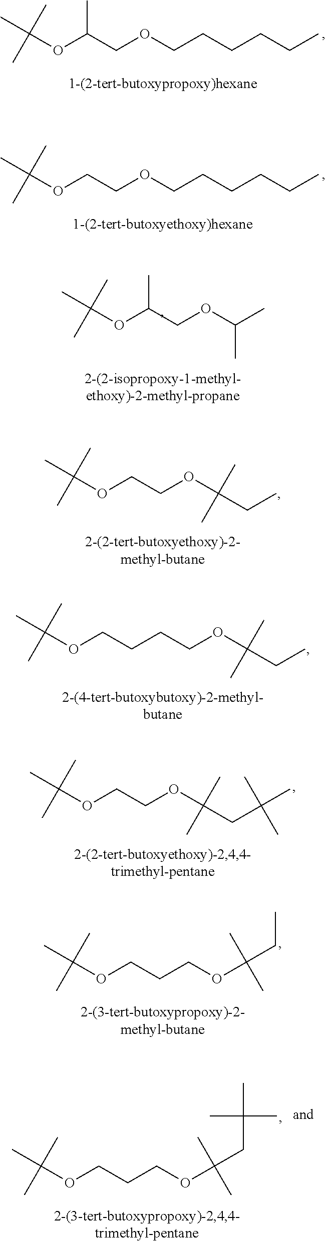

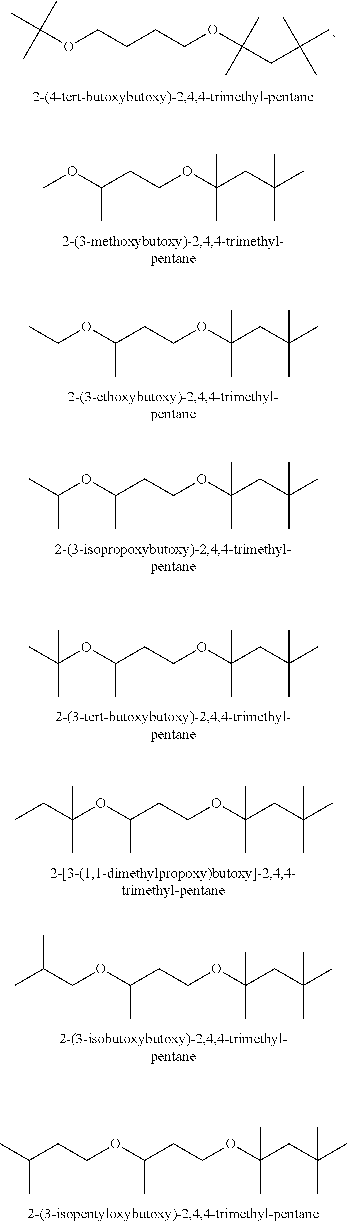

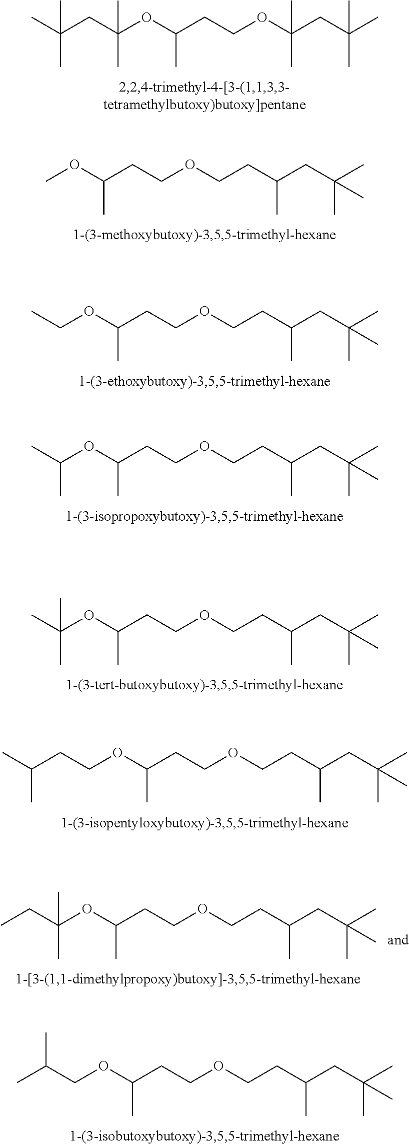

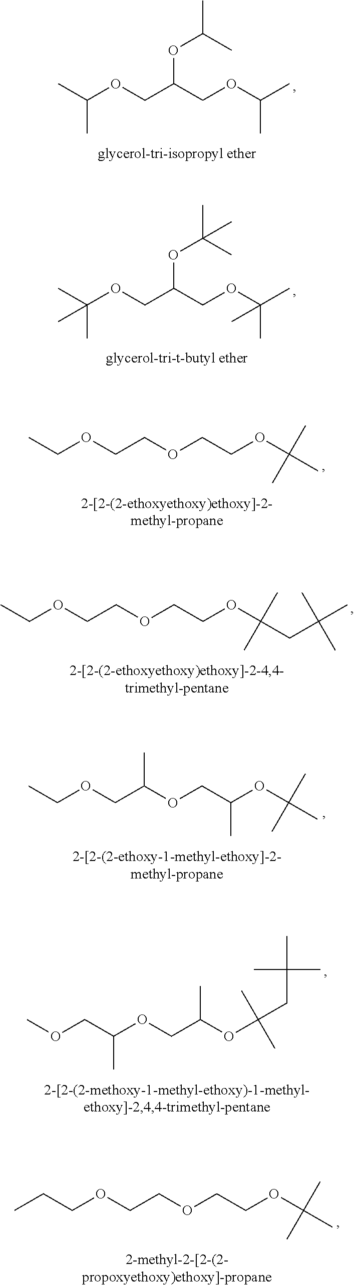

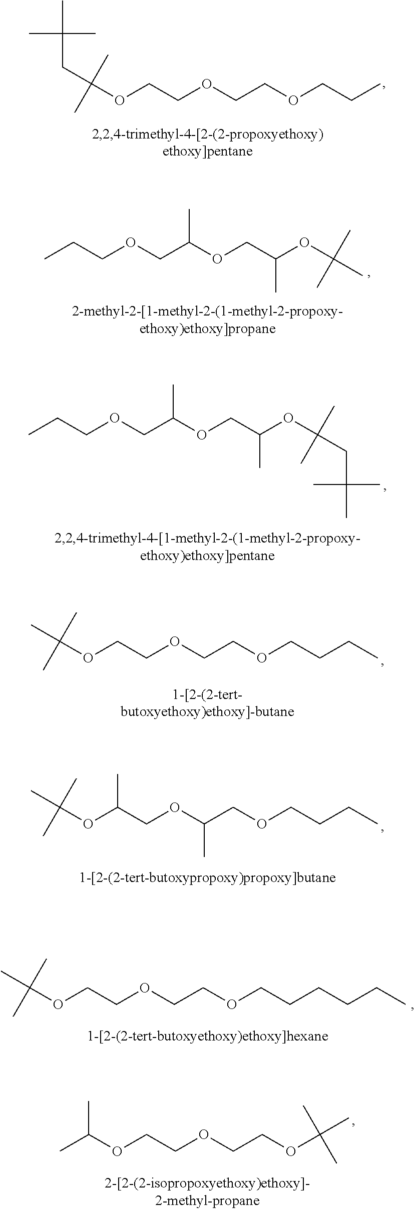

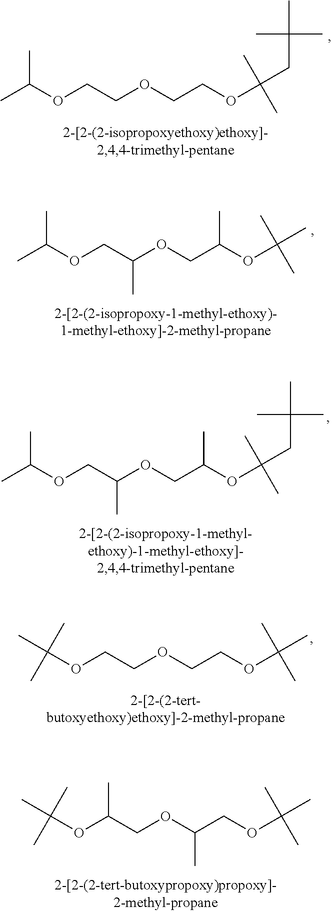

### Embodiment 28

The compound for use in cosmetic compositions as defined in any of the previous embodiments which is for the preparation of
a. topical compositions to be applied to keratinous substrate such as skin, lip, hair, nail;
b. antiperspirant and deodorant compositions in the form of spray, pencils, sticks, multiphase sticks, pastes, powder, aerosols, creams, cream foams, lotions, self-foaming, foam-like, after-foaming or foamable emulsions, gels, roll-on preparations, foams or depilatories;
c. skin care compositions such as lotions, creams, emulsions and microemulsions, moisturizers, anti-aging products, skin tightening lotions, anti-acne products, day-creams, night-cream, under eye-cream, face mask, face lotion, body lotion, after-shave lotions, cleaning milk, toners;
d. color cosmetic compositions such as lip-stick, pencils, lip-color, liquid lip color, lip stain, lip balm, lip-gloss, mascara, eye liner, eye-shadow, foundation, face powder, blush, peel cream, bb cream, cc cream, tinted moisturizer;
e. hair care compositions such as hair treatments, damage repair compositions, hair waxes, hair gels, hair foam, hair styling fluids, hair sprays, hair oil, mousse, shampoo, conditioner, hair remover creams;
f. sun care compositions such as sunscreens, after sun lotions, sun tanner, sunscreen oils, self-tanning products.
g. bath products such as bath soap, bath gel, cleansing milk, cleansing solution, make up remover, face wash.

### Embodiment 29

A cosmetic composition, comprising at least one compound of the formula (I) according to the previous embodiments, and at least one additional cosmetic ingredient.

### Embodiment 30

The cosmetic composition according to the previous embodiment, wherein the cosmetic composition is an aqueous composition.

### Embodiment 31

The cosmetic composition according to the previous embodiments, which comprises from 0.5 to 95 wt.-% of the at least one compound according to the previous embodiments of the formula (I), based on the total weight of the cosmetic composition.

### Embodiment 32

One or more compounds of the formula (I′):

R¹—O—R²(I′)
wherein
R¹ and R² are different from each other and are independently selected from the group of C1-C17 hydrocarbyl residues, said hydrocarbyl residues contain up to 8 CH₃ groups and up to 3 ether groups (—O—),
and wherein
the total number of carbon atoms of said compounds is 10 to 20,
the total number of ether groups of said compounds is 1 to 6, and
the total number of methyl groups (—CH₃) of said compounds is 1 to 13,
at least one of R¹ and R² is a branched C3-C17 hydrocarbyl residue,
with the proviso for all monoether compounds containing a total number of one ether group and containing a total number of 10 to 13 carbon atoms that the compounds are selected from the group consisting of 3,5,5-trimethyl-hexyl methyl ether, 3,5,5-trimethyl-hexyl ethyl ether, 3,5,5-trimethyl-hexyl n-propyl ether, 3,5,5-trimethyl-hexyl isopropyl ether, 3,5,5-trimethyl-hexyl 2-butyl ether, 3,5,5-trimethyl-hexyl isobutyl ether and 3,5,5-trimethyl-hexyl tert-butyl ether, n-pentyl-1,1,3,3-tetramethyl-butyl ether, n-butyl-1,1,3,3-tetramethyl-butyl ether, isoamyl-1,1,3,3-tetramethyl-butyl ether, isobutyl-1,1,3,3-tetramethyl-butyl ether,
and with the proviso for all monoether compounds containing a total number of one ether group and a total number of 14 to 20 carbon atoms that
either
   both R¹ and R² are independently selected from the group of C5-C9 hydrocarbyl residues, or
   R¹ is selected from C5-C17 linear hydrocarbyl residues and R² is selected from the group of branched C3-C9 hydrocarbyl residues, or
   one of the groups R¹ and R² is selected from linear C14-C17 hydrocarbyl residues, and with the exception of all monoether compounds containing at least one of R¹ and R² representing a 2-propyl-heptyl group,
and wherein the one or more compounds are selected from
   the monoethers 1-tert-butoxydecane and 1-tert-butoxydodecane,
   monoethers derived from di-isobutene, wherein R¹ is a 1,1,3,3-tetramethylbutyl group, and R² is selected from linear C7 to C12 alkyl groups or branched C6-C12 alkyl groups,
      monoethers derived from 3,5,5-trimethyl hexanol, wherein R¹ is a 3,5,5-trimethylhexyl group, and R² is selected from linear C2-C11 alkyl groups or branched C3-C11 alkyl groups, preferably a tert-butyl group, in particular the compounds of the structures

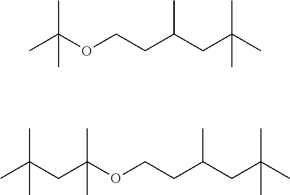

monoethers derived from di-isobutene, wherein R¹ is a 1,1,3,3-tetramethyl-butyl group, and R² is selected from n-butyl, n-pentyl, isobutyl and isoamyl groups,
monoethers derived from tri-isobutene, wherein R¹ is a 1,1,3,3,5,5-hexamethylhexyl group, and R² is selected from linear C2 to C8 alkyl groups or branched C3-C8 alkyl groups, in particular the compounds of the structures

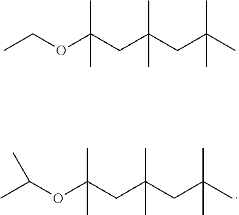

asymmetric diethers derived from ethylene oxide or propylene oxide, i.e. diethers containing ethylene or propylene groups as linker groups between the compounds' two ether —O— atoms, in particular the compounds of the structures

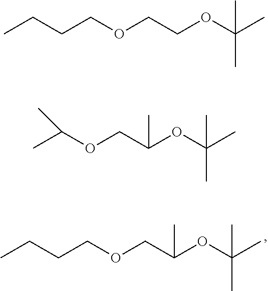

diethers derived from 1,3-butanediol, i.e. diethers containing 1,3-butylene groups as linker groups between the compounds' two ether —O— atoms, in particular the compound of the structure

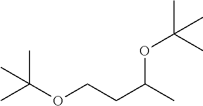

asymmetric diethers derived from 1,3-butanediol, i.e. diethers containing 1,3-butylene groups as linker groups between the compounds' two ether —O— atoms, wherein R¹ is a 1,1,3,3-tetramethylbutyl group, in particular the compounds of the structures

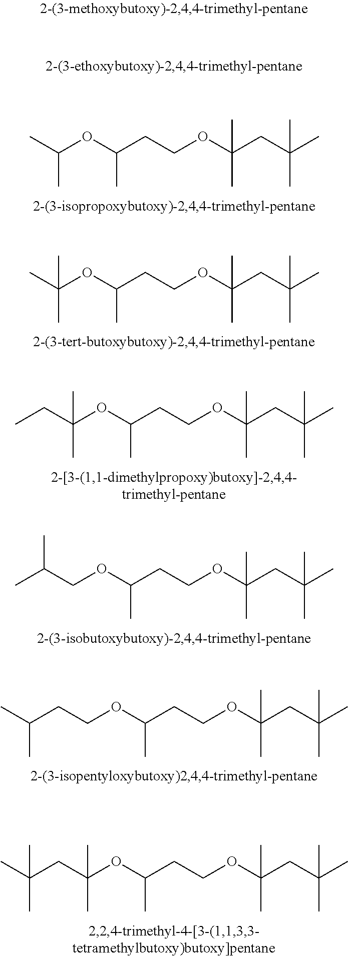

asymmetric diethers derived from 1,3-butanediol, i.e. diethers containing 1,3-butylene groups as linker groups between the compounds' two ether —O— atoms, wherein R¹ is a 3,5,5-trimethylhexyl group, in particular of the structures

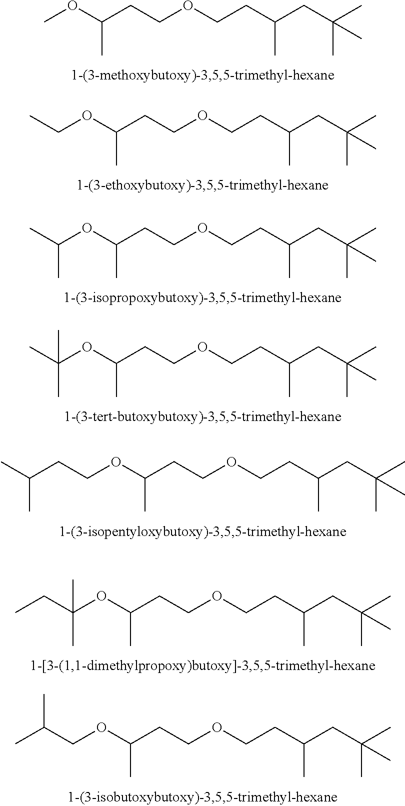

asymmetric triethers derived from ethylene oxide or propylene oxide, i.e. triethers exclusively containing alkylene groups selected from the group consisting of ethylene or propylene groups as linker groups between the compounds' three ether —O— atoms, more preferably containing either two ethylene groups or two propylene groups, in particular the compounds of the structures

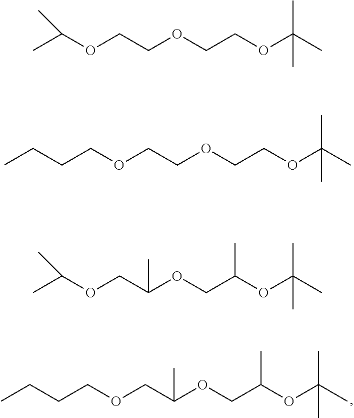

further preferably asymmetric triethers containing either two ethylene groups or two propylene groups as linker groups between the compounds' three ether —O— atoms, wherein one of the two terminal alkyl groups of the compound is a 1,1,3,3-tetramethylbutyl group or a 3,5,5-trimethylhexyl group.

### Embodiment 33

Use of one or more compounds of the formula (I) as defined above in cosmetic compositions.

### Embodiment 34

Use of one or more compounds of the formula (I″)

R¹—O—R²(I″)
wherein
R¹ and R² are different from each other and are independently selected from the group of C1-C17 hydrocarbyl residues, said hydrocarbyl residues contain up to 8 CH₃ groups and up to 3 ether groups (—O—), and
wherein
the total number of carbon atoms of said compounds is 14 to 20,
the total number of ether groups of said compounds is 1 to 6, and
the total number of methyl groups (—CH₃) of said compounds is 1 to 13,
at least one of R¹ and R² is a branched C3-C17 hydrocarbyl residue,
with the proviso for all monoether compounds that
either
   both R¹ and R² are independently selected from the group of C5-C9 hydrocarbyl residues, or
   R¹ is selected from C5-C17 linear hydrocarbyl residues and R² is selected from the group of branched C3-C9 hydrocarbyl residues, or
   one of the groups R¹ and R² is selected from linear C14-C17 hydrocarbyl residues,
   all monoether compounds containing at least one of R¹ and R² representing a 2-propyl-heptyl group are excepted,
and wherein the compounds are used as solvents, volatiles, spreading agents, emollients, delivery agents, active solubilizers, detackifiers or compatibilizers.

### Further Specific Aspects of the Invention

Another specific aspect of the present invention is related to cosmetic compositions comprising at least one compound of the formula (I):
R¹—O—R² (I)
   wherein
   R¹ and R² are different from each other and are independently selected from the group of C1-C17 hydrocarbyl residues,
   said hydrocarbyl residues contain up to 8 CH₃ groups, and wherein
   the total number of carbon atoms of said compound is 10 to 20,
   the total number of ether groups of said compound is 1, and
   the total number of methyl groups (—CH₃) of said compound is 1 to 13,
   at least one of R¹ and R² is a branched C3-C17 hydrocarbyl residue, with the proviso for all monoether compounds containing a total number of 10 to 13 carbon atoms that the compounds are selected from the group consisting of 1,1,3,3,-tetramethyl butyl ethers, preferred n-pentyl-1,1,3,3-tetramethyl-butyl ether, cyclopentyl 1,1,3,3-tetramethyl-butyl ether, n-butyl-1,1,3,3-tetramethyl-butyl ether, isoamyl-1,1,3,3-tetramethyl-butyl ether, isobutyl-1,1,3,3-tetramethyl-butyl ether,
   3,5,5-trimethyl-hexyl ethers, preferred 3,5,5-trimethyl-hexyl methyl ether, 3,5,5-trimethyl-hexyl ethyl ether, 3,5,5-trimethyl-hexyl n-propyl ether, 3,5,5-trimethyl-hexyl isopropyl ether, 3,5,5-trimethyl-hexyl n-butyl ether, 3,5,5-trimethyl-hexyl 2-butyl ether, 3,5,5-trimethyl-hexyl isobutyl ether and 3,5,5-trimethyl-hexyl tert-butyl ether,
   1,1,3,3,5,5-hexamethylhexyl-methyl ether,
   and the compounds 2-methylphenyl isopropyl ether, 3-methylphenyl isopropyl ether, 4-methylphenyl isopropyl ether, benzyl isopropyl ether, 3,5,5-trimethyl-hexyl 2-methyl-propyl ether, cyclohexyl 2-methyl-propyl ether, phenyl 2-methyl-propyl ether, 2-methylphenyl 2-methyl-propyl ether, 2-methylphenyl 2-methyl-propyl ether, 4-methylphenyl 2-methyl-propyl ether, benzyl 2-methyl-propyl ether, 3-methyl-butyl cyclopentyl ether, 3-methyl-butyl cyclohexyl ether, 3-methyl-butyl phenyl ether, 2-methylphenyl 3-methyl-butyl ether, 3-methylphenyl 3-methyl-butyl ether, 4-methylphenyl 3-methyl-butyl ether, benzyl 3-methyl-butyl ether, 1,1,3,3-tetramethyl-butyl 3-methyl-butyl ether, cyclohexyl tert-butyl ether, phenyl tert-butyl ether, 2-methylphenyl tert-butyl ether, 3-methylphenyl tert-butyl ether, 4-methylphenyl tert-butyl ether, cyclopentyl 1,1-dimethyl-propyl ether, cyclohexyl 1,1-dimethyl-propyl ether, phenyl 1,1-dimethyl-propyl ether, 2-methylphenyl 1,1-dimethyl-propyl ether, 3-methylphenyl 1,1-dimethyl-propyl ether, 4-methylphenyl 1,1-dimethyl-propyl ether, benzyl 1,1-dimethyl-propyl ether, cyclopentyl 2,2-dimethyl-propyl ether, cyclohexyl 2,2-dimethyl-propyl ether, phenyl 2,2-dimethyl-propyl ether, 2-methylphenyl 2,2-dimethyl-propyl ether, 3-methylphenyl 2,2-dimethyl-propyl ether, 4-methylphenyl 2,2-dimethyl-propyl ether, benzyl 2,2-dimethyl-propyl ether, cyclopentyl 3-methyl-pentyl ether, cyclohexyl 3-methyl-pentyl ether, phenyl 3-methyl-pentyl ether, 2-methylphenyl 3-methyl-pentyl ether, 3-methylphenyl 3-methyl-pentyl ether, 4-methylphenyl 3-methyl-pentyl ether, benzyl 3-methyl-pentyl ether, cyclopentyl 3,3-dimethyl-butyl ether, cyclohexyl 3,3-dimethyl-butyl ether, phenyl 3,3-dimethyl-butyl ether, 2-methylphenyl 3,3-dimethyl-butyl ether, 3-methylphenyl 3,3-dimethyl-butyl ether, 4-methylphenyl 3,3-dimethyl-butyl ether, benzyl 3,3-dimethyl-butyl ether, cyclopentyl 2,3-dimethyl-butyl ether, cyclohexyl 2,3-dimethyl-butyl ether, phenyl 2,3-dimethyl-butyl ether, 2-methylphenyl 2,3-dimethyl-butyl ether, 3-methylphenyl 2,3-dimethyl-butyl ether, 4-methylphenyl 2,3-dimethyl-butyl ether, benzyl 2,3-dimethyl-butyl ether, cyclopentyl 1,3-dimethyl-butyl ether, cyclohexyl 1,3-dimethyl-butyl ether, phenyl 1,3-dimethyl-butyl ether, 2-methylphenyl 1,3-dimethyl-butyl ether, 3-methylphenyl 1,3-dimethyl-butyl ether, 4-methylphenyl 1,3-dimethyl-butyl ether, benzyl 1,3-dimethyl-butyl ether,
   and with the proviso for all monoether compounds containing a total number of 14 to 20 carbon atoms that the compounds are selected from the group consisting of
   1,1,3,3,-tetramethyl butyl ethers, preferred n-hexyl 1,1,3,3-tetramethyl-butyl ether, n-heptyl 1,1,3,3-tetramethyl-butyl ether, n-octyl 1,1,3,3-tetramethyl-butyl ether, n-nonyl 1,1,3,3-tetramethyl-butyl ether, n-decyl 1,1,3,3-tetramethyl-butyl ether, n-undecyl 1,1,3,3-tetramethyl-butyl ether, n-dodecyl 1,1,3,3-tetramethyl-butyl ether, 3-methyl-pentyl 1,1,3,3-tetramethyl-butyl ether, 3,3-dimethyl-butyl 1,1,3,3-tetramethyl-butyl ether, 2,3-dimethyl-butyl 1,1,3,3-tetramethyl-butyl ether, 1,3-dimethyl-butyl 1,1,3,3-tetramethyl-butyl ether, 2,4,4-trimethyl-pentyl 1,1,3,3-tetramethyl-butyl ether, 3,5,5-trimethyl-hexyl 1,1,3,3-tetramethyl-butyl ether, 2-ethyl-hexyl 1,1,3,3-tetramethyl-butyl ether, cyclohexyl 1,1,3,3-tetramethyl-butyl ether, phenyl
   1,1,3,3-tetramethyl-butyl ether, 2-methylphenyl 1,1,3,3-tetramethyl-butyl ether, 3-methylphenyl 1,1,3,3-tetramethyl-butyl ether, 4-methylphenyl 1,1,3,3-tetramethyl-butyl ether, benzyl 1,1,3,3-tetramethyl-butyl ether, 1,1,3,3,5,5-hexamethylhexyl ethers, preferred n-pentyl 1,1,3,3,5,5-hexamethyl-hexyl ether, cyclopentyl 1,1,3,3,5,5-hexamethyl-hexyl ether, cyclohexyl 1,1,3,3,5,5-hexamethyl-hexyl ether, phenyl 1,1,3,3,5,5-hexamethyl-hexyl ether, 2-methylphenyl 1,1,3,3,5,5-hexamethyl-hexyl ether, 3-methylphenyl 1,1,3,3,5,5-hexamethyl-hexyl ether, 4-methylphenyl 1,1,3,3,5,5-hexamethyl-hexyl ether, benzyl 1,1,3,3,5,5-hexamethyl-hexyl ether, 1,1,3,3,5,5-hexamethylhexyl ethyl ether, 1,1,3,3,5,5-hexamethylhexyl-1-propyl ether, 1,1,3,3,5,5-hexamethylhexyl-n-propyl ether, 1,1,3,3,5,5-hexamethylhexyl-n-butyl ether, 1,1,3,3,5,5-hexamethylhexyl-1-butyl ether, 1,1,3,3,5,5-hexamethylhexyl-t-butyl ether, and 3,5,5-trimethyl hexyl ethers, preferred 3,5,5-trimethyl-hexyl n-pentyl ether, 3,5,5-trimethyl-hexyl n-hexyl ether, 3,5,5-trimethyl-hexyl n-heptyl ether, 3,5,5-trimethyl-hexyl n-octyl ether, 3,5,5-trimethyl-hexyl n-nonyl ether, 3,5,5-trimethyl-hexyl n-decyl ether, 3,5,5-trimethyl-hexyl n-undecyl ether,
   3,5,5-trimethyl-hexyl 3-methyl-butyl ether, 3,5,5-trimethyl-hexyl 1,1-dimethylpropyl ether, 3,5,5-trimethyl-hexyl 2,2-dimethylpropyl ether, 3,5,5-trimethyl-hexyl 3-methyl-pentyl ether, 3,5,5-trimethyl-hexyl 3,3-dimethyl-butyl ether, 3,5,5-trimethyl-hexyl 2,3-dimethyl-butyl ether, 3,5,5-trimethyl-hexyl 1,3-dimethyl-butyl ether, 3,5,5-trimethyl-hexyl 2,4,4-trimethyl-pentyl ether, 3,5,5-trimethyl-hexyl 2-ethyl-hexyl ether, 3,5,5-trimethyl-hexyl cyclopentyl ether, 3,5,5-trimethyl-hexyl cyclohexyl ether, 3,5,5-trimethyl-hexyl phenyl ether, 3,5,5-trimethyl-hexyl 2-methylphenyl ether, 3,5,5-trimethyl-hexyl 3-methylphenyl ether, 3,5,5-trimethyl-hexyl 4-methylphenyl ether, and 3,5,5-trimethyl-hexyl benzyl ether,
   and at least one additional cosmetic ingredient.

The compounds comprised by the cosmetic composition of this aspect of the invention can be applied in a beneficial way due to their volatility properties and sensory profile.

According to the invention, the cosmetic compositions according to the invention preferably comprise the monoethers containing a 1,1,3,3-tetramethylbutyl group or a 1,1,3,3,5,5-hexamethylhexyl group due to the volatility properties and sensory profile of these compounds.

In a preferred specific embodiment according to the invention, the monoether compounds containing a total number of 10 to 13 carbon atoms are selected from the group consisting of the 1,1,3,3,-tetramethyl butyl ethers n-pentyl-1,1,3,3-tetramethyl-butyl ether, cyclopentyl 1,1,3,3-tetramethyl-butyl ether, n-butyl-1,1,3,3-tetramethyl-butyl ether, isoamyl-1,1,3,3-tetramethyl-butyl ether, isobutyl-1,1,3,3-tetramethyl-butyl ether,
the 3,5,5-trimethyl-hexyl ethers 3,5,5-trimethyl-hexyl methyl ether, 3,5,5-trimethyl-hexyl ethyl ether, 3,5,5-trimethyl-hexyl n-propyl ether, 3,5,5-trimethyl-hexyl isopropyl ether, 3,5,5-trimethyl-hexyl n-butyl ether, 3,5,5-trimethyl-hexyl 2-butyl ether, 3,5,5-trimethyl-hexyl isobutyl ether and 3,5,5-trimethyl-hexyl tert-butyl ether,
1,1,3,3,5,5-hexamethylhexyl-methyl ether,
and the compounds 2-methylphenyl isopropyl ether, 3-methylphenyl isopropyl ether, 4-methylphenyl isopropyl ether, benzyl isopropyl ether, 3,5,5-trimethyl-hexyl 2-methyl-propyl ether, cyclohexyl 2-methyl-propyl ether, phenyl 2-methyl-propyl ether, 2-methylphenyl 2-methyl-propyl ether, 2-methylphenyl 2-methyl-propyl ether, 4-methylphenyl 2-methyl-propyl ether, benzyl 2-methyl-propyl ether, 3-methyl-butyl cyclopentyl ether, 3-methyl-butyl cyclohexyl ether, 3-methyl-butyl phenyl ether, 2-methylphenyl 3-methyl-butyl ether, 3-methylphenyl 3-methyl-butyl ether, 4-methylphenyl 3-methyl-butyl ether, benzyl 3-methyl-butyl ether, 1,1,3,3-tetramethyl-butyl 3-methyl-butyl ether, cyclohexyl tert-butyl ether, phenyl tert-butyl ether, 2-methylphenyl tert-butyl ether, 3-methylphenyl tert-butyl ether, 4-methylphenyl tert-butyl ether, cyclopentyl 1,1-dimethyl-propyl ether, cyclohexyl 1,1-dimethyl-propyl ether, phenyl 1,1-dimethyl-propyl ether, 2-methylphenyl 1,1-dimethyl-propyl ether, 3-methylphenyl 1,1-dimethyl-propyl ether, 4-methylphenyl 1,1-dimethyl-propyl ether, benzyl 1,1-dimethyl-propyl ether, cyclopentyl 2,2-dimethyl-propyl ether, cyclohexyl 2,2-dimethyl-propyl ether, phenyl 2,2-dimethyl-propyl ether, 2-methylphenyl 2,2-dimethyl-propyl ether, 3-methylphenyl 2,2-dimethyl-propyl ether, 4-methylphenyl 2,2-dimethyl-propyl ether, benzyl 2,2-dimethyl-propyl ether, cyclopentyl 3-methyl-pentyl ether, cyclohexyl 3-methyl-pentyl ether, phenyl 3-methyl-pentyl ether, 2-methylphenyl 3-methyl-pentyl ether, 3-methylphenyl 3-methyl-pentyl ether, 4-methylphenyl 3-methyl-pentyl ether, benzyl 3-methyl-pentyl ether, cyclopentyl 3,3-dimethyl-butyl ether, cyclohexyl 3,3-dimethyl-butyl ether, phenyl 3,3-dimethyl-butyl ether, 2-methylphenyl 3,3-dimethyl-butyl ether, 3-methylphenyl 3,3-dimethyl-butyl ether, 4-methylphenyl 3,3-dimethyl-butyl ether, benzyl 3,3-dimethyl-butyl ether, cyclopentyl 2,3-dimethyl-butyl ether, cyclohexyl 2,3-dimethyl-butyl ether, phenyl 2,3-dimethyl-butyl ether, 2-methylphenyl 2,3-dimethyl-butyl ether, 3-methylphenyl 2,3-dimethyl-butyl ether, 4-methylphenyl 2,3-dimethyl-butyl ether, benzyl 2,3-dimethyl-butyl ether, cyclopentyl 1,3-dimethyl-butyl ether, cyclohexyl 1,3-dimethyl-butyl ether, phenyl 1,3-dimethyl-butyl ether, 2-methylphenyl 1,3-dimethyl-butyl ether, 3-methylphenyl 1,3-dimethyl-butyl ether, 4-methylphenyl 1,3-dimethyl-butyl ether, benzyl 1,3-dimethyl-butyl ether,
and the monoether compounds containing a total number of 14 to 20 carbon atoms are selected from the group consisting of
the 1,1,3,3,-tetramethyl butyl ethers n-hexyl 1,1,3,3-tetramethyl-butyl ether, n-heptyl 1,1,3,3-tetramethyl-butyl ether, n-octyl 1,1,3,3-tetramethyl-butyl ether, n-nonyl 1,1,3,3-tetramethyl-butyl ether, n-decyl 1,1,3,3-tetramethyl-butyl ether, n-undecyl 1,1,3,3-tetramethyl-butyl ether, n-dodecyl 1,1,3,3-tetramethyl-butyl ether, 3-methyl-pentyl 1,1,3,3-tetramethyl-butyl ether, 3,3-dimethyl-butyl 1,1,3,3-tetramethyl-butyl ether, 2,3-dimethyl-butyl 1,1,3,3-tetramethyl-butyl ether, 1,3-dimethyl-butyl 1,1,3,3-tetramethyl-butyl ether, 2,4,4-trimethyl-pentyl 1,1,3,3-tetramethyl-butyl ether, 3,5,5-trimethyl-hexyl 1,1,3,3-tetramethyl-butyl ether, 2-ethyl-hexyl 1,1,3,3-tetramethyl-butyl ether, cyclohexyl 1,1,3,3-tetramethyl-butyl ether, phenyl 1,1,3,3-tetramethyl-butyl ether, 2-methylphenyl 1,1,3,3-tetramethyl-butyl ether, 3-methylphenyl 1,1,3,3-tetramethyl-butyl ether, 4-methylphenyl 1,1,3,3-tetramethyl-butyl ether, benzyl 1,1,3,3-tetramethyl-butyl ether,
the 1,1,3,3,5,5-hexamethylhexyl ethers n-pentyl 1,1,3,3,5,5-hexamethyl-hexyl ether,
cyclopentyl 1,1,3,3,5,5-hexamethyl-hexyl ether, cyclohexyl 1,1,3,3,5,5-hexamethyl-hexyl ether, phenyl 1,1,3,3,5,5-hexamethyl-hexyl ether, 2-methylphenyl 1,1,3,3,5,5-hexamethyl-hexyl ether, 3-methylphenyl 1,1,3,3,5,5-hexamethyl-hexyl ether, 4-methylphenyl 1,1,3,3,5,5-hexamethyl-hexyl ether, benzyl 1,1,3,3,5,5-hexamethyl-hexyl ether, 1,1,3,3,5,5-hexamethylhexyl ethyl ether, 1,1,3,3,5,5-hexamethylhexyl-1-propyl ether, 1,1,3,3,5,5-hexamethylhexyl-n-propyl ether, 1,1,3,3,5,5-hexamethylhexyl-n-butyl ether, 1,1,3,3,5,5-hexamethylhexyl-1-butyl ether, 1,1,3,3,5,5-hexamethylhexyl-t-butyl ether,
and the 3,5,5-trimethyl hexyl ethers 3,5,5-trimethyl-hexyl n-pentyl ether, 3,5,5-trimethyl-hexyl n-hexyl ether, 3,5,5-trimethyl-hexyl n-heptyl ether, 3,5,5-trimethyl-hexyl n-octyl ether, 3,5,5-trimethyl-hexyl n-nonyl ether, 3,5,5-trimethyl-hexyl n-decyl ether, 3,5,5-trimethyl-hexyl n-undecyl ether, 3,5,5-trimethyl-hexyl 3-methyl-butyl ether, 3,5,5-trimethyl-hexyl 1,1-dimethylpropyl ether, 3,5,5-trimethyl-hexyl 2,2-dimethylpropyl ether, 3,5,5-trimethyl-hexyl 3-methyl-pentyl ether, 3,5,5-trimethyl-hexyl 3,3-dimethyl-butyl ether, 3,5,5-trimethyl-hexyl 2,3-dimethyl-butyl ether, 3,5,5-trimethyl-hexyl 1,3-dimethyl-butyl ether, 3,5,5-trimethyl-hexyl 2,4,4-trimethyl-pentyl ether, 3,5,5-trimethyl-hexyl 2-ethyl-hexyl ether, 3,5,5-trimethyl-hexyl cyclopentyl ether, 3,5,5-trimethyl-hexyl cyclohexyl ether, 3,5,5-trimethyl-hexyl phenyl ether, 3,5,5-trimethyl-hexyl 2-methylphenyl ether, 3,5,5-trimethyl-hexyl 3-methylphenyl ether, 3,5,5-trimethyl-hexyl 4-methylphenyl ether, and 3,5,5-trimethyl-hexyl benzyl ether.

Further preferably, the the monoether compounds containing a total number of 10 to 13 carbon atoms are selected from the group consisting of
the 1,1,3,3,-tetramethyl butyl ethers n-pentyl-1,1,3,3-tetramethyl-butyl ether, cyclopentyl 1,1,3,3-tetramethyl-butyl ether, n-butyl-1,1,3,3-tetramethyl-butyl ether, isoamyl-1,1,3,3-tetramethyl-butyl ether, isobutyl-1,1,3,3-tetramethyl-butyl ether,
and the monoether compounds containing a total number of 14 to 20 carbon atoms are selected from the group consisting of
the 1,1,3,3,-tetramethyl butyl ethers n-hexyl 1,1,3,3-tetramethyl-butyl ether, n-heptyl 1,1,3,3-tetramethyl-butyl ether, n-octyl 1,1,3,3-tetramethyl-butyl ether, n-nonyl 1,1,3,3-tetramethyl-butyl ether, n-decyl 1,1,3,3-tetramethyl-butyl ether, n-undecyl 1,1,3,3-tetramethyl-butyl ether, n-dodecyl 1,1,3,3-tetramethyl-butyl ether, 3-methyl-pentyl 1,1,3,3-tetramethyl-butyl ether, 3,3-dimethyl-butyl 1,1,3,3-tetramethyl-butyl ether, 2,3-dimethyl-butyl 1,1,3,3-tetramethyl-butyl ether, 1,3-dimethyl-butyl 1,1,3,3-tetramethyl-butyl ether, 2,4,4-trimethyl-pentyl 1,1,3,3-tetramethyl-butyl ether, 3,5,5-trimethyl-hexyl 1,1,3,3-tetramethyl-butyl ether, 2-ethyl-hexyl 1,1,3,3-tetramethyl-butyl ether, cyclohexyl 1,1,3,3-tetramethyl-butyl ether, phenyl 1,1,3,3-tetramethyl-butyl ether, 2-methylphenyl 1,1,3,3-tetramethyl-butyl ether, 3-methylphenyl 1,1,3,3-tetramethyl-butyl ether, 4-methylphenyl 1,1,3,3-tetramethyl-butyl ether, and benzyl 1,1,3,3-tetramethyl-butyl ether.

Also preferably, the monoether compounds containing a total number of 10 to 13 carbon atoms are selected from 1,1,3,3,5,5-hexamethylhexyl-methyl ether,
and the monoether compounds containing a total number of 14 to 20 carbon atoms are selected from the group consisting of the 1,1,3,3,5,5-hexamethylhexyl ethers n-pentyl 1,1,3,3,5,5-hexamethyl-hexyl ether, cyclopentyl 1,1,3,3,5,5-hexamethyl-hexyl ether, cyclohexyl 1,1,3,3,5,5-hexamethyl-hexyl ether, phenyl 1,1,3,3,5,5-hexamethyl-hexyl ether, 2-methylphenyl 1,1,3,3,5,5-hexamethyl-hexyl ether, 3-methylphenyl 1,1,3,3,5,5-hexamethyl-hexyl ether, 4-methylphenyl 1,1,3,3,5,5-hexamethyl-hexyl ether, benzyl 1,1,3,3,5,5-hexamethyl-hexyl ether, 1,1,3,3,5,5-hexamethylhexyl ethyl ether, 1,1,3,3,5,5-hexamethylhexyl-1-propyl ether, 1,1,3,3,5,5-hexamethylhexyl-n-propyl ether, 1,1,3,3,5,5-hexamethylhexyl-n-butyl ether, 1,1,3,3,5,5-hexamethylhexyl-1-butyl ether and 1,1,3,3,5,5-hexamethylhexyl-t-butyl ether.

Also further preferably, the monoether compounds containing a total number of 10 to 13 carbon atoms are selected from the group consisting of the 3,5,5-trimethyl-hexyl ethers 3,5,5-trimethyl-hexyl methyl ether, 3,5,5-trimethyl-hexyl ethyl ether, 3,5,5-trimethyl-hexyl n-propyl ether, 3,5,5-trimethyl-hexyl isopropyl ether, 3,5,5-trimethyl-hexyl n-butyl ether, 3,5,5-trimethyl-hexyl 2-butyl ether, 3,5,5-trimethyl-hexyl isobutyl ether and 3,5,5-trimethyl-hexyl tert-butyl ether,
and the monoether compounds containing a total number of 14 to 20 carbon atoms are selected from the group consisting of the 3,5,5-trimethyl hexyl ethers 3,5,5-trimethyl-hexyl n-pentyl ether, 3,5,5-trimethyl-hexyl n-hexyl ether, 3,5,5-trimethyl-hexyl n-heptyl ether, 3,5,5-trimethyl-hexyl n-octyl ether, 3,5,5-trimethyl-hexyl n-nonyl ether, 3,5,5-trimethyl-hexyl n-decyl ether, 3,5,5-trimethyl-hexyl n-undecyl ether,
3,5,5-trimethyl-hexyl 3-methyl-butyl ether, 3,5,5-trimethyl-hexyl 1,1-dimethylpropyl ether, 3,5,5-trimethyl-hexyl 2,2-dimethylpropyl ether, 3,5,5-trimethyl-hexyl 3-methyl-pentyl ether, 3,5,5-trimethyl-hexyl 3,3-dimethyl-butyl ether, 3,5,5-trimethyl-hexyl 2,3-dimethyl-butyl ether, 3,5,5-trimethyl-hexyl 1,3-dimethyl-butyl ether, 3,5,5-trimethyl-hexyl 2,4,4-trimethyl-pentyl ether, 3,5,5-trimethyl-hexyl 2-ethyl-hexyl ether, 3,5,5-trimethyl-hexyl cyclopentyl ether, 3,5,5-trimethyl-hexyl cyclohexyl ether, 3,5,5-trimethyl-hexyl phenyl ether, 3,5,5-trimethyl-hexyl 2-methylphenyl ether, 3,5,5-trimethyl-hexyl 3-methylphenyl ether, 3,5,5-trimethyl-hexyl 4-methylphenyl ether, and 3,5,5-trimethyl-hexyl benzyl ether.

In a preferred embodiment according to the invention, the cosmetic composition according to the invention comprises at least one compound of the formula (I) wherein
R¹ and R² are different from each other and independently selected from C1-C17 linear, branched cycloaliphatic or aromatic hydrocarbon residues, which contain 0-8, preferred 0-7, more preferred 0-6, even more preferred 0-5, even further preferred 0-3, or preferred 1-8, more preferred 1-7, even more preferred 1-6, further preferred 1-5, even further preferred 1-3, and specifically preferred 1, 2, 3, 4, 5, 6, 7 CH₃ groups,
the total number of carbon atoms of said compounds is 10-20, preferred 10-17, more preferred 10-15, or preferred 11-15, even more preferred 11-13, and specifically preferred 11, 12, 13, 14, 15,
the total number of methyl groups (—CH₃) of said compounds is 1-13, preferred 1-12, more preferred 1-10, even more preferred 1-9, further preferred 1-7, even further preferred 1-6, even more further preferred 1-4, or preferred 2-6, more preferred 2-4, and specifically preferred 1, 2, 3, 4, 5, 6, 7, 8, 9, 10, 11, 12, or 13.

In another preferred embodiment according to the invention, the at least one compound of the formula (I) comprised by the cosmetic composition according to the invention is selected from the group consisting of:

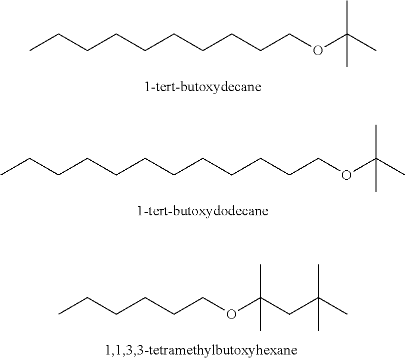

and the further group of mono-ethers consisting of

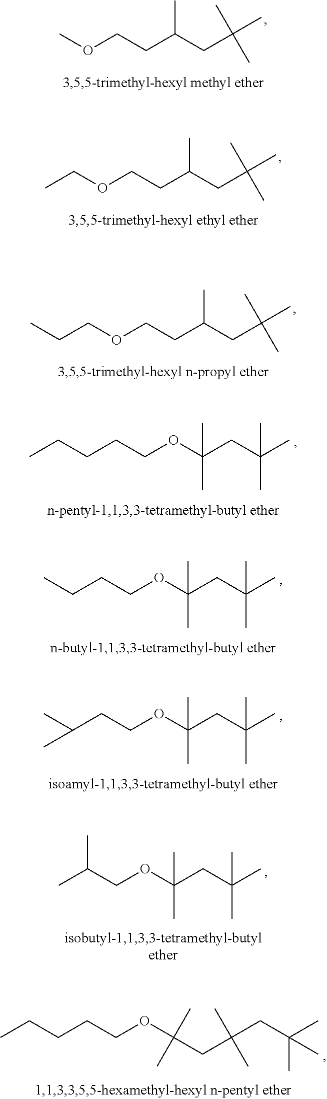

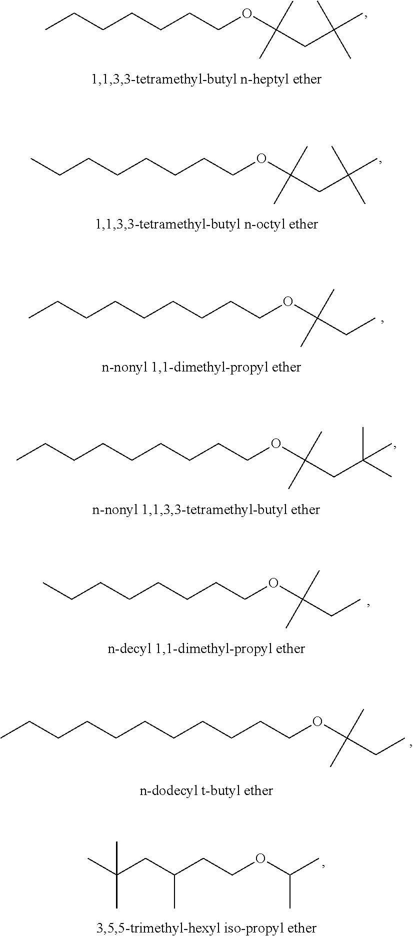

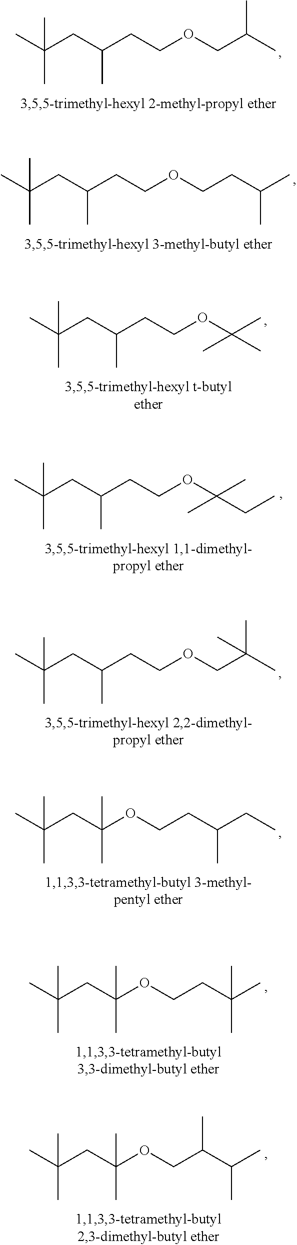

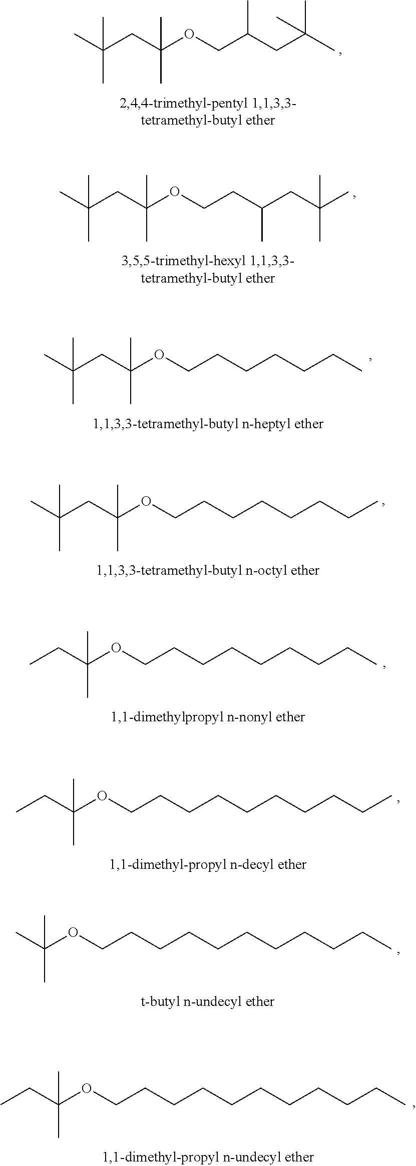

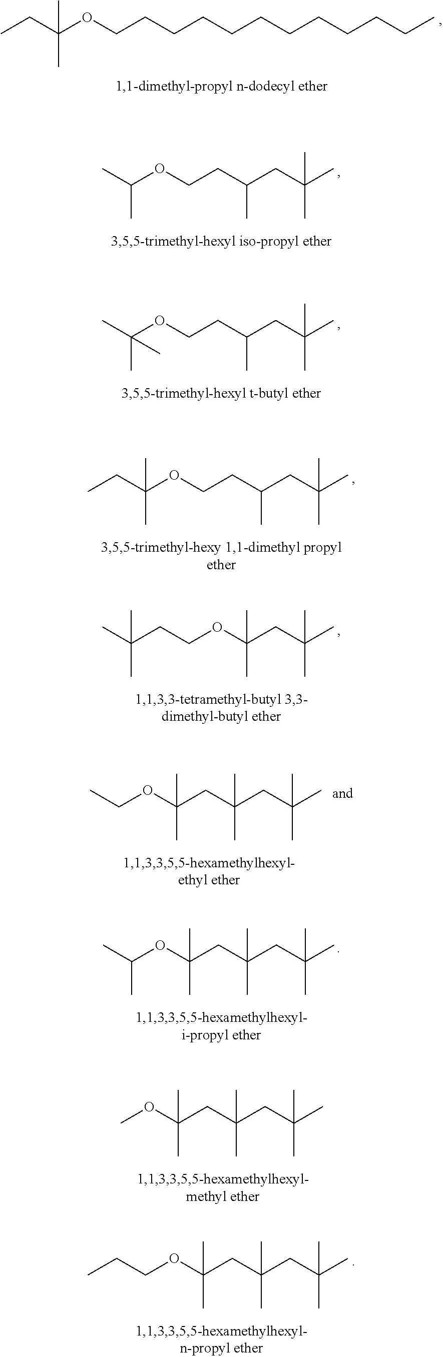

In a further embodiment according to the invention, the at least one compound of the formula (I) comprised by the cosmetic composition according to the invention is selected from the group consisting of:

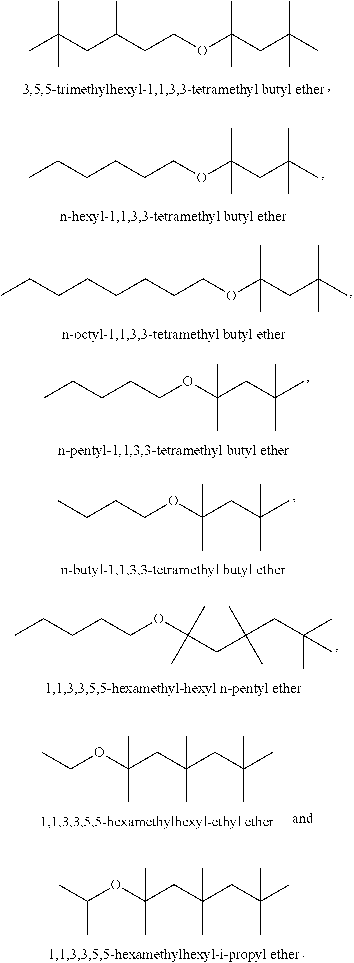

According to the embodiment, the compound of the formula (I) is preferably selected from n-pentyl-1,1,3,3-tetramethyl butyl ether or n-butyl-1,1,3,3-tetramethyl butyl ether.

The compounds according to the embodiment have superior properties, in particular regarding regarding volatility, contact angle, surface tension, preading properties and sensory properties, and are thus beneficially applied in the formulation of cosmetic compositions, improving the sensory properties and rheological properties of the chemical compositions as well.

In another preferred embodiment according to the invention, the cosmetic composition according to the invention contains at least 0.01 weight-% of the compound of the formula (I) based on the total weight of the composition, more preferably 1 to 95 wt-%, even more preferably 2 to 90 weight-%, and most preferably 5 to 80 weight-% based on the total weight of the composition.

The preferred ranges of the content of the compound of the formula (I) as described above depend on the type of cosmetic formulation. In the following table, the preferred ranges of the content of the compound of the formula (I) are displayed for several specific types of cosmetic compositions:

| | Typical range % |
|---|---|
| Eye makeup | |
| Eyebrow pencil | 10-30 |
| Eyeliners | 10-60 |
| Eye shadow | 1-50 |
| Eye lotion | 5-60 |
| Eye make-up remover | 5-85 |
| Mascara | 2-50 |
| Hair Care | |
| Conditioners | 0.1-90 |
| Sprays/aerosol fixatives | 0.1-50 |
| Straighteners | 0.1-50 |
| Shampoos | 0.1-50 |
| Dyes and colors | 0.1-20 |
| Hair oils | 10-80 |
| Makeup | |
| Foundations | 1-60 |
| Makeup base, primer | 5-60 |
| Lipstick | 10-50 |
| Makeup fixatives | 1-50 |
| Personal hygiene products | |
| Deodorants | 2-90 |
| Aftershave lotion | 2-50 |
| Preshave lotions | 2-50 |
| Skin care products | |
| Face and neck lotions | 1-90 |
| Body and hand lotions | 1-80 |
| Moisturizers | 1-80 |
| Night creams and lotions | 1-90 |
| Skin fresheners | 1-60 |
| Sun Care products | |
| Suntan gels, creams | 10-60 |
| Sunscreens | 1-50 |

In still another preferred embodiment according to the invention, the cosmetic composition according to the invention is an aqueous composition, and wherein preferably the water content of the cosmetic composition is in the range of 10 to 80 wt-%, more preferably 15 to 70 weight-% of the composition, most preferably 20 to 60 weight-% based on the total weight of the composition. As described above, the water content of the cosmetic composition strongly depends on the type of the cosmetic composition.

In a further preferred embodiment according to the invention, the cosmetic composition is a skin care formulation.

According to this embodiment, the skin care formulation is preferably a lotion, a cream or an emulsion, more preferably selected from under eye cream, day cream, night cream, and makeup removal gel cream.

The skin care formulation according to this embodiment preferably is a skin care formulation comprising 0.1 to 90 wt-%, more preferably 2 to 80 wt-%, even more preferably 5 to 70 wt-%, and most preferably 10 to 60 wt % of the at least one compound according to the formula (I) based on the total weight of the cosmetic composition.

Further, in the skin care formulation according to this embodiment, the at least one compound of the formula (I) is preferably selected from n-pentyl-1,1,3,3-tetramethyl butyl ether or n-butyl-1,1,3,3-tetramethyl butyl ether.

In another preferred embodiment according to the invention, the cosmetic composition is a color cosmetic composition.

According to this embodiment, the color cosmetic composition is preferably a lip stick, mascara, bb cream, and eye liner.

The color cosmetic composition according to this embodiment preferably is a color cosmetic composition comprising 1 to 90 wt-%, more preferably 2 to 80 wt-%, even more preferably 3 to 70 wt-%, and most preferably 5 to 60 wt-% of the at least one compound according to the formula (I) based on the total weight of the cosmetic composition.

Further, in the color cosmetic composition according to this embodiment, the at least one compound of the formula (I) is preferably selected from n-pentyl-1,1,3,3-tetramethyl butyl ether or n-butyl-1,1,3,3-tetramethyl butyl ether.

In yet another preferred embodiment according to the invention, the cosmetic composition is a hair care formulation.

According to this embodiment, the hair care formulation is preferably selected from hair oils, and hair sprays, in particular leave-in-hair conditioner spray.

The hair care formulation according to this embodiment preferably is a hair care formulation comprising 0.01 to 99 wt-%, more preferably 0.5 to 95 wt-%, even more preferably 1 to 92 wt-%, and most preferably 2 to 90 wt-% of the at least one compound according to the formula (I) based on the total weight of the cosmetic composition

Further, in the hair care formulation according to this embodiment, the at least one compound of the formula (I) is preferably selected from n-pentyl-1,1,3,3-tetramethyl butyl ether or n-butyl-1,1,3,3-tetramethyl butyl ether.

In a further preferred embodiment according to the invention, the cosmetic composition according to the invention is selected from the following table and comprises at least one compound of the formula (I), preferably selected from n-pentyl-1,1,3,3-tetramethyl butyl ether or n-butyl-1,1,3,3-tetramethyl butyl ether, in the following quantities indicated therein:

| | Typical range % |
|---|---|
| Eye makeup | |
| Eyebrow pencil | 10-30, preferred 10-20 and 20-30 |
| Eyeliners | 10-60, preferred 10-35 and 35-60 |
| Eye shadow | 1-50, preferred 1-25 and 25-50 |
| Eye lotion | 5-60, preferred 5-30 and 30-60 |
| Eye make-up remover | 5-85, preferred 5-40 and 40-85 |
| Mascara | 2-50, preferred 2-25 and 25-50 |
| Hair Care | |
| Conditioners | 0.1-90, preferred 0, 1-45 and 45-90 |
| Sprays/aerosol fixatives | 0.1-50, preferred 0, 1-25 and 25-50 |
| Straighteners | 0.1-50, preferred 0, 1-25 and 25-50 |
| Shampoos | 0.1-50, preferred 0, 1-25 and 25-50 |
| Dyes and colors | 0.1-20, preferred 0, 1-10 and 10-20 |
| Hair oils | 10-80, preferred 10-40 and 40-80 |
| Makeup | |
| Foundations | 1-60, preferred 1-30 and 30-60 |
| Makeup base, primer | 5-60, preferred 5-30 and 30-60 |
| Lipstick | 10-50, preferred 10-30 and 30-50 |
| Makeup fixatives | 1-50, preferred 1-25 and 25-50 |
| Personal hygiene products | |
| Deodorants | 2-90, preferred 2-45 and 45-90 |
| Aftershave lotion | 2-50, preferred 2-25 and 25-50 |
| Preshave lotions | 2-50, preferred 2-25 and 25-50 |
| Skin care products | |
| Face and neck lotions | 1-90, preferred 1-45 and 45-90 |
| Body and hand lotions | 1-80, preferred 1-40 and 40-80 |
| Moisturizers | 1-80, preferred 1-40 and 40-80 |
| Night creams and lotions | 1-90, preferred 1-45 and 45-90 |
| Skin fresheners | 1-60, preferred 1-30 and 30-60 |
| Sun Care products | |
| Suntan gels, creams | 10-60, preferred 10-35 and 35-60 |
| Sunscreens | 1-50, preferred 1-25 and 25-50 |

In a preferred embodiment according to the invention, the cosmetic composition according to the invention comprises at least one further ingredient, preferably at least two further ingredients, more preferably at least three further ingredients selected from oils, waxes, thickeners, humectants, sunscreens, emollients, fats obtained from animals, or minerals, in particular metal oxides, organic compounds acting as colorants, fragrances or preservatives, pigments, natural products and mixtures thereof obtained by extraction of plants, processed plant parts or polymers, emulsifiers and surfactants.

The present invention also relates to one or more compounds of the formula (I′):

R¹—O—R² (I′)
wherein
R¹ and R² are different from each other and are independently selected from the group of C1-C17 hydrocarbyl residues,
said hydrocarbyl residues contain up to 8 CH₃ groups and up to 3 ether groups (—O—),
and wherein
the total number of carbon atoms of said compounds is 10 to 20,
the total number of ether groups of said compounds is 1, and
the total number of methyl groups (—CH₃) of said compounds is 1 to 13,
at least one of R¹ and R² is a branched C3-C17 hydrocarbyl residue,
and wherein the one or more compounds are selected from
monoethers derived from tri-isobutene, wherein R¹ is a 1,1,3,3,5,5-hexamethylhexyl group, and R² is selected from linear C2 to C8 alkyl groups or branched C3-C8 alkyl groups, in particular the compounds of the structure

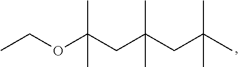

wherein the compound

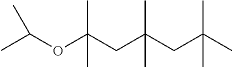 [SMILES: CC(C)OC(C)(C)CC(C)(C)CC(C)(C)C]

is excluded.

Due to their beneficial properties, the above-cited compounds can be beneficially applied as solvents, spreading agents, emollients, delivery agents, active solubilizers, detackifiers or compatibilizers.

Preferred examples of the compounds of the formula (I′) of this embodiment of the invention are pentyl 1,1,3,3,5,5-hexamethyl-hexyl ether, cyclopentyl 1,1,3,3,5,5-hexamethyl-hexyl ether, cyclohexyl 1,1,3,3,5,5-hexamethyl-hexyl ether, phenyl 1,1,3,3,5,5-hexamethyl-hexyl ether, 2-methylphenyl 1,1,3,3,5,5-hexamethyl-hexyl ether, 3-methylphenyl 1,1,3,3,5,5-hexamethyl-hexyl ether, 4-methylphenyl 1,1,3,3,5,5-hexamethyl-hexyl ether, benzyl 1,1,3,3,5,5-hexamethyl-hexyl ether, and 1,1,3,3,5,5-hexamethylhexyl ethyl ether, wherein 1,1,3,3,5,5-hexamethyl-hexyl n-pentyl ether, 1,1,3,3,5,5-hexamethylhexyl-ethyl ether and 1,1,3,3,5,5-hexamethylhexyl hexyl ether are most preferred.

The present invention further relates to the use of one or more compounds of the formula (I) according to the invention for cosmetic compositions.

Therein, the one or more compounds of the formula (I) according to the invention are preferably n-pentyl-1,1,3,3-tetramethyl butyl ether or n-butyl-1,1,3,3-tetramethyl butyl ether.

In a preferred embodiment of the invention, the one or more compounds of the formula (I) according to the invention as described above is used in a cosmetic composition which is a is a cosmetic formulation for skin care, hair care, sun care, an antiperspirant or deodorant, or a color cosmetic composition.

In a further preferred embodiment of the invention, the one or more compounds of the formula (I) according to the invention as described above is used in a cosmetic composition, wherein the cosmetic composition contains at least 0.01 weight-% of the compound of the formula (I) based on the total weight of the composition, more preferably 1 to 95 wt-%, even more preferably 2 to 90 weight-%, and most preferably 5 to 80 weight-% based on the total weight of the composition.

In a further preferred embodiment of the invention, the one or more compounds of the formula (I) according to the invention as described above is used in a cosmetic composition, wherein the compound is used as a solvent, volatile, spreading agent, emollient, delivery agent, active solubilizer, detackifier or compatibilizer.

Preferably, according to this embodiment of the invention, the one or more compounds of the formula (I) is used as a volatile in a cosmetic composition.

Further, the present invention relates to the use of one or more compounds of the formula (I″)

R¹—O—R² (I″)
wherein
R¹ and R² are different from each other and are independently selected from the group of C1-C17 hydrocarbyl residues, said hydrocarbyl residues contain up to 8 CH₃ groups and up to 3 ether groups (—O—), and
wherein
the total number of carbon atoms of said compounds is 14 to 20,
the total number of ether groups of said compounds is 1, and
the total number of methyl groups (—CH₃) of said compounds is 1 to 13,
at least one of R¹ and R² is a branched C3-C17 hydrocarbyl residue,
with the proviso for all monoether compounds that
either
   both R¹ and R² are independently selected from the group of C5-C9 hydrocarbyl residues, or
   R¹ is selected from C5-C17 linear hydrocarbyl residues and R² is selected from the group of branched C3-C9 hydrocarbyl residues, or
   one of the groups R¹ and R² is selected from linear C14-C17 hydrocarbyl residues,
   all monoether compounds containing at least one of R¹ and R² representing a 2-propyl-heptyl group are excepted, and wherein the compounds are used as solvents, spreading agents, emollients, delivery agents, active solubilizers, detackifiers or compatibilizers.

### EXAMPLES

The following Tab. 1 summarizes the ether derivatives synthesized in the Examples.

TABLE 1
Derivatives synthesized

| | | terminal | | | 2,4,4-trimethyl- |
|---|---|---|---|---|---|
| | | alcohol | | | pentan-2-ol |
| alcohol | isopropyl alcohol | isobutyl alcohol | isoamyl alcohol | t-butyl alcohol | (di-isobutene |
| core | C3 | C4 | C5 | C4 | based) C8 |
| 1,3 propanediol | | Example 7 | Example 6 | | |
| C3 | | | | | |
| 1,4 butanediol | Example 8 | Example 5 | Example 9 | Example 3 | |
| C4 | | | | | |
| 1,5 propanediol | Example 10 | Example 11 | Example 12 | | |
| C5 | | | | | |
| 1,6 hexanediol | Example 4 | Example 13 | Example 14 | | |
| C6 | | | | | |
| 1-butanol C4 | | | | | Example 21 |
| 1-pentanol C5 | | | | | Example 20 |
| 1-hexanol C6 | | | | | Example 18 |
| 1-octanol C8 | | | | | Example 19 |
| 1-decanol C10 | | Example 15 | | Example 1 | |
| 1-dodecanol | Example 16 | | | Example 2 | |
| C12 | | | | | |
| Isoamyl alcohol | | | | | Example 22 |
| C5 | | | | | |
| 3,5,5-trimethyl- | | | | | Example 17 |
| hexan-1-ol C9 | | | | | |

The following Tab. 2 summarizes the boiling points of the ethers (° C./mbar) as obtained in the Examples according to the invention

TABLE 2
Boiling points of the Example compounds

| | | terminal | | | 2,4,4-trimethyl- |
|---|---|---|---|---|---|
| | | alcohol | | | pentan-2-ol |
| alcohol | isopropyl alcohol | isobutyl alcohol | isoamyl alcohol | t-butyl alcohol | (di-isobutene |
| core | C3 | C4 | C5 | C4 | based) C8 |
| 1,3 propanediol | | 90-92/22 | 123-124/22 | | |
| C3 | | | | | |
| 1,4 butanediol | 82-83/22 | 106-108/22 | 137-139/22 | 77-78/6 | |
| C4 | | | | | |
| 1,5 propanediol | 96/22 | 128/26 | 150-153/22 | | |
| C5 | | | | | |
| 1,6 hexanediol | 110-111/22 | 135-136/22 | 165/22 | | |
| C6 | | | | | |
| 1-butanol C4 | | | | | 62/6 |
| 1-pentanol C5 | | | | | 78-80/7 |
| 1-hexanol C6 | | | | | 90-91/5 |
| 1-octanol C8 | | | | | 125/9 |
| 1-decanol C10 | | 134-136/7 | | 118-120/11 | |
| 1-dodecanol | 149-151/9 | | | 130-132/6 | |
| C12 | | | | | |
| Isoamyl alcohol | | | | | 70-72/6 |
| C5 | | | | | |
| 3,5,5- | | | | | 122-127/4 |
| trimethylhexan- | | | | | |
| 1-ol C9 | | | | | |

### Example 1

### Synthesis of n-decyl-t-butyl ether

In a 500 ml three-necked bottle, equipped with refluxing condenser, thermometer, mechanical stirrer, dropping funnel and gas outlet tube 100 g (0.63 mol) 1-decanol, 234 g (3.16 mol) t-butanol, 12.4 g (0.13 mol) concentrated sulfuric acid and 76 g (0.63 mol) anhydrous MgSO₄ were mixed at room temperature under a nitrogen atmosphere and heated to 80° C. for 7 h. In the course of this reaction a second portion of t-butanol (150 g) was added after 5 hrs. The MgSO₄ was removed by filtration. Afterwards, 112 g NaHCO₃ were added to neutralize the batch, the solid was removed by filtration and the liquid phase subjected to a vacuum distillation. 71 g of a fraction boiling at 121-126° C. at 20 mbar and consisting of n-decanol and n-decyl-t-butyl ether in a ratio 61.4:38.6 (as determined by GC analysis) were obtained. 42 g of this fraction were placed in a 250 ml three necked bottle equipped with refluxing condenser, thermometer, magnetic stirrer, dropping funnel, vacuum valve and heated to 60° C. at 560 mbar. 53.87 g oleic acid chloride were added and the reaction continued for 2 h. The complete esterification was determined by means of GC. After neutralization with NaHCO₃ and filtration, fractional distillation yielded 6.5 g of n-decyl-t-butyl ether (b.p. 118-120° C. at 11 mbar; 99.8% purity as determined by GC analysis).

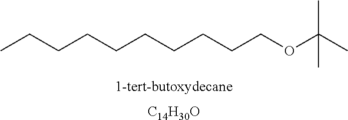

### Example 2

### Synthesis of n-dodecyl-t-butyl ether

In a 1000 ml three-necked bottle, equipped with refluxing condenser, thermometer, mechanical stirrer, dropping funnel and gas outlet tube 100 g (0.54 mol) 1-decanol, 199 g (2.68 mol) t-butanol, 10.5 g (0.11 mol) concentrated sulfuric acid and 64.6 g (0.54 mol) anhydrous MgSO₄ were mixed at room temperature under a nitrogen atmosphere and heated to 80° C. for 7 h. After cooling to room temperature, the MgSO₄ was removed by filtration and 2 g concentrated sulfuric acid, 228 g t-butanol and 64.4 g anhydrous MgSO₄ were added. The mixture was heated to 80° C. and maintained at this temperature for 7 h. The MgSO₄ was removed by filtration. Afterwards, 150 g NaHCO₃ were added to neutralize the batch, the solid was removed by filtration and the liquid phase was subjected to a distillation under reduced pressure. 32.3 g of a fraction boiling at 125-130° C./5 mbar and consisting of n-dodecanol and n-dodecyl-t-butyl ether in a ratio 31.9:68.1 (as determined by GC analysis) were obtained. The fraction was placed in a 250 ml three necked bottle equipped with refluxing condenser, thermometer, magnetic stirrer, dropping funnel, vacuum valve and heated to 60° C. at 580 mbar. 18.3 g oleic acid chloride were added and the reaction was continued for 3 h. The complete esterification of the alcohol starting material was determined by means of GC. After neutralization with NaHCO₃ and filtration, fractional distillation yielded 6.4 g of n-dodecyl-t-butyl ether (b.p. 130-132° C. at 6 mbar; 100% purity as determined by GC analysis).

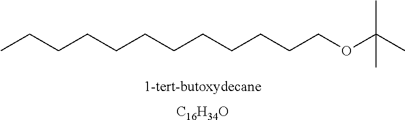 [SMILES: CCCCCCCCCCCCOC(C)(C)C ; "C16H34O"]

### Example 3

### Synthesis of 1,4-butanediol-di-t-butyl ether

Example 3a Synthesis of 1,4-butanediol-mono-t-butyl ether (Synthesis intermediate, not according to the invention) In a 1000 ml three-necked bottle, equipped with refluxing condenser, thermometer, magnetic stirrer, dropping funnel and gas outlet tube 100 g (1.11 mol) 1,4-butanediol, 500 g (6.75 mol) t-butanol and 18 g (0.22 mol) Amberlyst 15 (sulphonic acid modified ion exchange resin) were mixed at room temperature under a nitrogen atmosphere and heated to 63° C. for 2 h. The mixture was cooled to room temperature and afterwards the Amberlyst 15 ion exchange resin was filtered off.

The above-described procedure was repeated on identical scale.

Both batches were combined and neutralized with NaHCO₃.

Distillation under reduced pressure yielded 138.7 g of a mono-t-butyl ether rich fraction boiling at 80-85° C./6 mbar.

Composition of the fraction (as determined by GC analysis): 1,4-butanediol:mono-t-butyl ether:di-t-butyl ether=3:95:2

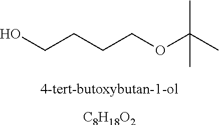

### Example 3b Synthesis of 1,4-butanediol-di-t-butyl ether

In a 1000 ml three-necked bottle, equipped with refluxing condenser, thermometer, mechanical stirrer, dropping funnel and gas outlet tube 136.3 g (0.93 mol) of the 1,4-butanediol-mono-t-butyl ether fraction from example 4a 411 g (5.55 mol) t-butanol, 21.8 g (0.22 mol) concentrated sulfuric acid and 160 g (1.34 mol) anhydrous MgSO₄ were mixed at room temperature under a nitrogen atmosphere and heated to 76° C. for 7 h. In the course of this reaction, a second portion of t-butanol (120 g) was added after 3 h. The ratio 1,4-butanediol-mono-t-butyl ether:di-t-butyl ether was 52.5:47.5 (as determined by GC analysis). The MgSO₄ was removed by filtration. Afterwards, 227 g NaHCO₃ were added to neutralize the batch, and the solid was removed by filtration. Low boiling compounds, such as water and t-butanol, were removed at 30° C. at 20 mbar.

Yield: 91.8 g of a high boiling fraction having the composition (as determined by GC analysis) 1,4-butanediol:mono-t-butyl ether:di-t-butyl ether=8.1:46.4:45.5

In a 500 ml three-necked bottle, equipped with refluxing condenser, thermometer, magnetic stirrer, dropping funnel and vacuum valve the 91.8 g (0.46 mol OH) of the 1,4-butanediol-di-t-butyl ether enriched fraction and 152.3 g (0.51 mol) oleic acid chloride were placed at room temperature. The pressure was adjusted to 500 mbar, and the reaction was continued for 7 h. The completion of the esterification reaction was determined by means of GC. After neutralization with NaHCO₃ (70 g) and filtration, fractional distillation yielded 8.3 g of 1,4-butanediol-di-t-butyl ether (b.p. 77-78° C. at 6 mbar; 97.56% as determined to GC analysis).

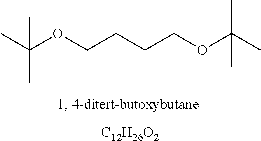

### Example 4

### Synthesis of 1,6-hexanediol-di-isopropyl ether

In a 500 ml three-necked bottle, equipped with refluxing condenser, thermostat, thermometer, magnetic stirrer, dropping funnel and gas outlet tube 300 g (4.99 mol) 2-propanol were placed and heated to 60° C. 7.29 g (0.32 mol) sodium was added in thin slices over 10 minutes. The mixture was heated to 80° C. and maintained at this temperature until the sodium was dissolved (2 h). The mixture was cooled to 60° C. Afterwards, 30.18 g (0.12 mol) 1,6-dibromohexane and 0.05 g KI were added, and the mixture was heated to 80° C. for 4 h. After a few minutes, a solid started to precipitate.

The solid was filtered off and the liquid phase was mixed with 1400 ml DI water, 370 g NaCl and 100 ml n-hexane. The upper organic layer was separated and the water layer once again extracted with 100 ml n-hexane. The organic layers were combined (300 ml) and dried over NaCl.

This reaction sequence was repeated two times in identical scale (in total three batches). Distillation of the combined organic phases of the three batches under reduced pressure yielded 26 g 1,6-hexanediol-di-isopropyl ether (b.p. 110-111° C. at 22 mbar; purity 99.5% as determined by GC analysis)

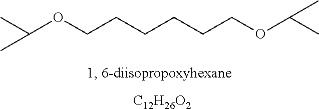

### Example 5

### Synthesis of 1,4-butanediol-di-isobutyl ether

In a 500 ml three-necked bottle, equipped with refluxing condenser, thermostat, thermometer, magnetic stirrer, dropping funnel and gas outlet tube 300 g (4.05 mol) iso-butyl alcohol were placed and heated to 60° C. 7.29 g (0.32 mol) sodium was added in thin slices over 15 minutes. The temperature increased to 90° C. during the addition, and the reaction mixture was maintained at this temperature until the sodium was dissolved (1 h). The mixture was cooled to 60° C. Then, 26.28 g (0.12 mol) 1,4-dibromobutane and 0.05 g KI were added, and the mixture was heated to 80° C. for 4 h. After a few minutes a solid started to precipitate.

The solid was filtered off and the liquid phase was mixed with 1400 ml DI water, 185 g NaCl and 100 ml n-hexane. The upper organic layer was separated, and the aqueous layer was extracted once more with 100 ml n-hexane. The organic layers were combined (500 ml) and dried over NaCl.

This reaction sequence was repeated two times in identical scale (in total three batches). Distillation of the combined organic phases of the three batches under reduced pressure yielded 24.5 g 1,4-butanediol-di-isobutyl ether (b.p. 106-108° C. at 22 mbar; purity of 99.5% as determined by GC analysis).

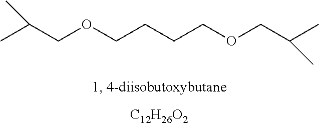

### Example 6

### Synthesis of 1,3-propanediol-di-isopentyl ether

In a 500 ml three-necked bottle, equipped with refluxing condenser, thermostat, thermometer, magnetic stirrer, dropping funnel and gas outlet tube 300 g (3.40 mol) iso-pentyl alcohol were placed and heated to 60° C. 7.29 g (0.32 mol) sodium was added in thin slices over 15 minutes. The temperature increased to 84° C. during the addition, was increased to 105° C. and then maintained at this temperature until the sodium was dissolved (1 h). The mixture was cooled to 60° C. Afterwards, 22.47 g (0.12 mol) 1,3-dibromopropane and 0.05 g KI were added, and the mixture was heated to 80° C. for 4 h. After a few minutes a solid started to precipitate.

The solid was filtered off, and liquid phase mixed with 1400 ml DI water, 420 g NaCl and 100 ml n-hexane. The upper organic layer was separated and the water layer once again extracted with 100 ml n-hexane. The organic layers were combined (520 ml) and dried over NaCl.

This reaction sequence was repeated five times on identical scale (in total six batches). Distillation of the combined organic phases of the six batches under reduced pressure yielded 11.3 g 1,3-propanediol-di-isopentyl ether (b.p. 123-124° C. at 22 mbar; purity of 97.9% as determined by GC analysis).

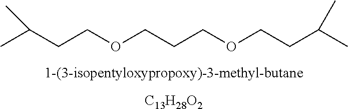

### Examples 7 to 14

The following tables 3 and 4 summarize the structures and the key reaction data for the synthesis of additional di-ethers derived from 1,3-propanediol, 1,4-butanediol, 1,5-pentanediol and 1,6-hexanediol having isopropyl, isobutyl and isopentyl terminal groups.

TABLE 3
Structures of the products of Examples 7-14

| ex- | |
|---|---|
| ample | structure |
| 7 | 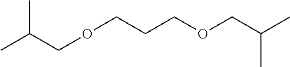 |
| | 1-(3-isobutoxypropoxy)-2-methyl-propane |
| 8 | 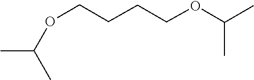 |
| | 1,4-diisopropoxybutane |
| 9 | 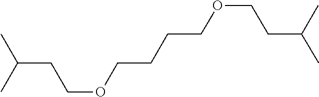 [SMILES: CC(C)CCOCCCCOCCC(C)C] |
| | 1-(4-isopentyloxybutoxy)-3-methyl-butane |
| 10 | 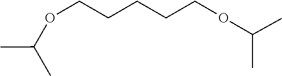 |
| | 1,5-diisopropoxypentane |
| 11 | 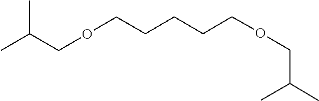 |
| | 1,5-diisobutoxypentane |
| 12 | 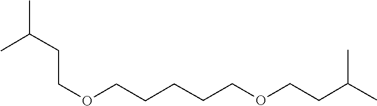 [SMILES: CC(C)CCOCCCCCOCCC(C)C] |
| | 1,5-diisopentyloxypentane |
| 13 | 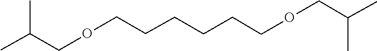 [SMILES: CC(C)COCCCCCCOCC(C)C] |
| | 1,6-diisobutoxyhexane |
| 14 | 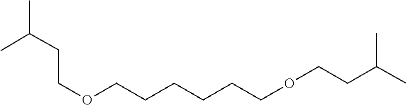 [SMILES: CC(C)CCOCCCCCCOCCC(C)C] |
| | 1,6-diisopentyloxyhexane |

TABLE 4
Key reaction data of Examples 7 to 14

| | | Repeats | | | | | |
|---|---|---|---|---|---|---|---|
| | reaction | (number of | di-bromo | | yield | b.p. | purity |
| example | protocol | batches) | compound | alcohol | [g] | ° C./mbar | [% GC] |
| 7 | In full | 0 | di-Br-propane | isobutyl | 4.0 | 90-92/22 | 92.3 |
| | analogy to | | | | | | |
| | example 5 | | | | | | |
| 8 | In full | 3 | di-Br-butane | isopropyl | 20.0 | 82-83/22 | 100 |
| | analogy to | | | | | | |
| | example 4 | | | | | | |
| 9 | example 6 | 3 | di-Br-butane | isopentyl | 31.9 | 137-139/22 | 100 |
| 10 | example 4 | 3 | di-Br-pentane | isopropyl | 19.1 | 96/22 | 99.7 |
| 11 | example 5 | 3 | di-Br-pentane | isobutyl | 35.7 | 128/26 | 99.0 |
| 12 | example 6 | 3 | di-Br-pentane | isopentyl | 49.7 | 150-153/22 | 99.1 |
| 13 | example 5 | 3 | di-Br-hexane | isobutyl | 41.5 | 135-136/22 | 98.3 |
| 14 | example 6 | 3 | di-Br-hexane | isopentyl | 33.0 | 165/22 | 98.0 |

### Example 15

### Synthesis of n-decyl-isobutyl ether

In a 500 ml three-necked bottle, equipped with refluxing condenser, thermostat, thermometer, magnetic stirrer, dropping funnel and gas outlet tube 259 g (3.48 mol) iso-butyl alcohol were placed and heated to 60° C. 7.29 g (0.32 mol) sodium was added in thin slices over 15 minutes.

The temperature increased to 90° 0 during the addition, and the reaction mixture was maintained at this temperature until the sodium was dissolved (1 h). The mixture was cooled to 60° C. Then, 53 g (0.12 mol) 1-bromodecane and 0.05 g KI were added, and the mixture was heated to 80° C. for 12 h. After a few minutes a solid started to precipitate.

The solid was filtered off and the liquid phase was mixed with 1400 ml DI water, 500 g NaCl and 100 ml n-hexane. The upper organic layer was separated, and the aqueous layer was extracted once more with 100 ml n-hexane. The organic layers were combined (500 ml) and dried over NaCl. This reaction sequence was repeated two times in identical scale (in total three batches).

Distillation of the combined organic phases of the three batches under reduced pressure yielded 116.6 g n-Decyl-isobutyl ether (b.p. 134-136° C. at 7 mbar; purity of 99.9% as determined by GO analysis).

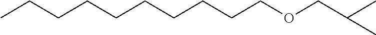

### Example 16

### Synthesis of n-dodecyl-isopropyl ether

In a 500 ml three-necked bottle, equipped with refluxing condenser, thermostat, thermometer, magnetic stirrer, dropping funnel and gas outlet tube 208 g (3.48 mol) iso-propyl alcohol were placed and heated to 60° C. 7.29 g (0.32 mol) sodium was added in thin slices over 15 minutes. The temperature increased to 90° C. during the addition, and the reaction mixture was maintained at this temperature until the sodium was dissolved (1 h). The mixture was cooled to 60° C. Then, 59.8 g (0.12 mol) 1-bromododecane and 0.05 g KI were added, and the mixture was heated to 80° C. for 12 h. After a few minutes a solid started to precipitate.

The solid was filtered off and the liquid phase was mixed with 1400 ml DI water, 500 g NaCl and 100 ml n-hexane. The upper organic layer was separated, and the aqueous layer was extracted once more with 100 ml n-hexane. The organic layers were combined (500 ml) and dried over NaCl. This reaction sequence was repeated two times in identical scale (in total three batches).

Distillation of the combined organic phases of the three batches under reduced pressure yielded 120.3 g n-Dodecyl-isopropyl ether (b.p. 149-151° C. at 9 mbar; purity of 99.5% as determined by GC analysis).

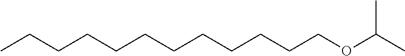 [SMILES: CCCCCCCCCCCCOC(C)C]

### Example 17

### Synthesis of 3,5,5-trimethylhexyl-1,1,3,3-tetramethyl butyl ether

In a 250 ml three-necked bottle, equipped with refluxing condenser, thermometer, magnetic stirrer 40 g (0.2772 mol) 3,5,5-trimethyl-hexanol and 31.12 g (0.2772 mol) diisobutylene were mixed at room temperature. 7 g (0.0714 mol) concentrated sulfuric acid were added slowly within 10 minutes. The temperature was increased to 107-112° C. for 44 hrs. After cooling to room temperature 60 g NaHCO₃ were added to neutralize the batch. Solids were removed from the liquid fraction by filtration.

The reaction was repeated twice (in total three batches).

The three liquid fractions were unified and subjected to a vacuum distillation. 17.8 g of a fraction boiling at 122-127° C./4 mbar and consisting of 3,5,5-trimethylhexyl-1,1,3,3-tetramethyl butyl ether (87% by means of GC) and minor portions of tri-isobutylene and tetra-isobutylene were obtained.

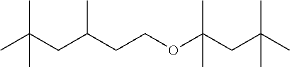 [SMILES: CC(CCOC(C)(C)CC(C)(C)C)CC(C)(C)C]

### Example 18

### Synthesis of n-hexyl-1,1,3,3-tetramethyl butyl ether

In a 1000 ml three-necked bottle, equipped with refluxing condenser, thermometer and mechanical stirrer the following three consecutive runs were carried under N₂:

| | stock | | | Amberlyst |
|---|---|---|---|---|
| run | solution* (g) | di-isobutylene** (g) | n-hexanol (g) | 15*** (g) |
| 1 | 385 | | 54.8 | 119 |
| 2 | 385 | | 54.8 | 119 |
| 3 | | 229 | 202 | 119 |

*mixture consisting of 45.7% n-hexanol and 54.3% di-isobutylene
**mixture consisting of approx. 75% 2,4,4-trimethyl-1-pentene and 25% 2,4,4-trimethyl-2-pentene
***sulphonic acid functionalized ion exchange resin H⁺ form; prior to use washed twice with 2-propanol and dried in vacuum

For run 1 the stock solution, the n-hexanol and the Amberlyst 15 were mixed, the temperature adjusted to 28° C. and maintained for 8 hrs. The liquid reaction mixture was separated from the catalyst by filtration.

The wet catalyst was transferred back to the reaction bottle. Stock solution and n-hexanol for run 2 were added. The temperature was adjusted to 28° C. and maintained for 8 hrs. The liquid reaction mixture was separated from the catalyst by filtration.

The wet catalyst was transferred back to the reaction bottle. Di-isobutylene and n-hexanol for run 3 were added. The temperature was adjusted to 28° C. and maintained for 8 hrs. The liquid reaction mixture was separated from the catalyst by filtration.

The liquid reaction mixtures from the runs 1 to 3 were unified and neutralized with 100 g NaHCO₃.

After filtration they were subjected to a distillation under reduced pressure yielding 95.7 g n-hexyl-1,1,3,3-tetramethyl butyl ether (b.p. 90-91° C. at 5 mbar; purity 95.4% as determined by GC analysis; target product accompanied by minor portions of tri-isobutylene and tetra-isobutylene isomers).

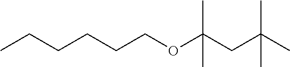

### Example 19

### Synthesis of n-octyl-1,1,3,3-tetramethyl butyl ether

In a 1000 ml three-necked bottle, equipped with refluxing condenser, thermometer and mechanical stirrer the following three consecutive runs were carried under N₂:

| run | di-isobutylene** (g) | n-octanol (g) | Amberlyst 15*** (g) |
|---|---|---|---|
| 1 | 200 | 232 | 70 |
| 2 | 200 | 232 | 70 |
| 3 | 200 | 232 | 70 |

**mixture consisting of approx. 75% 2,4,4-trimethyl-1-pentene and 25% 2,4,4-trimethyl-2-pentene
***sulphonic acid functionalized ion exchange resin H⁺ form; prior to use washed twice with 2-propanol and dried in vacuum

For run 1 the di-isobutylene, the n-octanol and the Amberlyst 15 were mixed, the temperature adjusted to 28° C. and maintained for 8 hrs. The liquid reaction mixture was separated from the catalyst by filtration.

The wet catalyst was transferred back to the reaction bottle. Di-isobutylene and n-octanol for run 2 were added. The temperature was adjusted to 28° C. and maintained for 8 hrs. The liquid reaction mixture was separated from the catalyst by filtration.

The wet catalyst was transferred back to the reaction bottle. Di-isobutylene and n-octanol for run 3 were added. The temperature was adjusted to 28° C. and maintained for 8 hrs. The liquid reaction mixture was separated from the catalyst by filtration.

The liquid reaction mixtures from the runs 1 to 3 were unified and neutralized with 100 g NaHCO₃.

After filtration they were subjected to a distillation under reduced pressure yielding 32 g n-octyl-1,1,3,3-tetramethyl butyl ether (b.p. 125° C. at 9 mbar; purity 98.45% as determined by GC analysis; target product accompanied by minor portions of n-octanol and isobutylene oligomers).

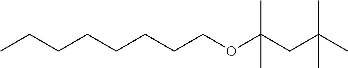 [SMILES: CCCCCCCCOC(C)(C)CC(C)(C)C]

### Example 20

### Synthesis of n-pentyl-1,1,3,3-tetramethyl butyl ether

In a 1000 ml three-necked bottle, equipped with refluxing condenser, thermometer and mechanical stirrer the following three consecutive runs were carried under N:

| run | di-isobutylene** (g) | n-pentanol (g) | Amberlyst 15*** (g) |
|---|---|---|---|
| 1 | 230 | 181 | 76 |
| 2 | 230 | 181 | 76 |
| 3 | 230 | 181 | 76 |

**mixture consisting of approx. 75% 2,4,4-trimethyl-1-pentene and 25% 2,4,4-trimethyl-2-pentene
***sulphonic acid functionalized ion exchange resin H⁺ form; prior to use washed twice with 2-propanol and dried in vacuum

For run 1 the di-isobutylene, the n-pentanol and the Amberlyst 15 were mixed, the temperature adjusted to 28° C. and maintained for 8 hrs. The liquid reaction mixture was separated from the catalyst by filtration.

The wet catalyst was transferred back to the reaction bottle. Di-isobutylene and n-pentanol for run 2 were added. The temperature was adjusted to 28° C. and maintained for 8 hrs. The liquid reaction mixture was separated from the catalyst by filtration.

The wet catalyst was transferred back to the reaction bottle. Di-isobutylene and n-pentanol for run 3 were added. The temperature was adjusted to 28° C. and maintained for 8 hrs. The liquid reaction mixture was separated from the catalyst by filtration.

The liquid reaction mixtures from the runs 1 to 3 were unified and neutralized with 140 g NaHCO₃.

After filtration they were subjected to a distillation under reduced pressure yielding 31.4 g n-pentyl-1,1,3,3-tetramethyl butyl ether (b.p. 78-80° C. at 7 mbar; purity 97.7% as determined by GC analysis; target product accompanied by minor portions of isobutylene oligomers).

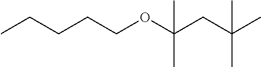

### Example 21

### Synthesis of n-butyl-1,1,3,3-tetramethyl butyl ether

In a 1000 ml three-necked bottle, equipped with refluxing condenser, thermometer and mechanical stirrer the following three consecutive runs were carried under N₂:

| run | di-isobutylene** (g) | n-butanol (g) | Amberlyst 15*** (g) |
|---|---|---|---|
| 1 | 260 | 171.7 | 84.7 |
| 2 | 260 | 171.7 | 84.7 |
| 3 | 260 | 171.7 | 84.7 |

**mixture consisting of approx. 75% 2,4,4-trimethyl-1-pentene and 25% 2,4,4-trimethyl-2-pentene
***sulphonic acid functionalized ion exchange resin H⁺ form; prior to use washed twice with 2-propanol and dried in vacuum

For run 1 the di-isobutylene, the n-butanol and the Amberlyst 15 were mixed, the temperature adjusted to 28° C. and maintained for 8 hrs. The liquid reaction mixture was separated from the catalyst by filtration.

The wet catalyst was transferred back to the reaction bottle. Di-isobutylene and n-butanol for run 2 were added. The temperature was adjusted to 28° C. and maintained for 8 hrs. The liquid reaction mixture was separated from the catalyst by filtration.

The wet catalyst was transferred back to the reaction bottle. Di-isobutylene and n-butanol for run 3 were added. The temperature was adjusted to 28° C. and maintained for 8 hrs. The liquid reaction mixture was separated from the catalyst by filtration.

The liquid reaction mixtures from the runs 1 to 3 were unified and neutralized with 145 g NaHCO₃.

After filtration they were subjected to a distillation under reduced pressure yielding 23.9 g n-butyl-1,1,3,3-tetramethyl butyl ether (b.p. 62° C. at 6 mbar; purity 99.5% as determined by GC analysis; target product accompanied by minor portions of isobutylene oligomers).

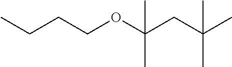

### Example 22

### Synthesis of isoamyl-1,1,3,3-tetramethyl butyl ether

In a 2000 ml three-necked bottle, equipped with refluxing condenser, thermometer and mechanical stirrer the following run was carried under N₂:

| run | di-isobutylene** (g) | isoamyl alcohol (g) | Amberlyst 15*** (g) |
|---|---|---|---|
| 1 | 600 | 471 | 178 |

**mixture consisting of approx. 75% 2,4,4-trimethyl-1-pentene and 25% 2,4,4-trimethyl-2-pentene
***sulphonic acid functionalized ion exchange resin H⁺ form; prior to use washed twice with 2-propanol and dried in vacuum

The di-isobutylene, the isoamyl alcohol and the Amberlyst 15 were mixed, the temperature adjusted to 25° C. and maintained for 30 hrs. The liquid reaction mixture was separated from the catalyst by filtration.

The liquid reaction mixture was neutralized with 30 g NaHCO₃.

After filtration it was subjected to a distillation under reduced pressure yielding 17.2 g isoamyl-1,1,3,3-tetramethyl butyl ether (b.p. 70-72° C. at 6 mbar; purity 91.6% as determined by GC analysis; target product accompanied by minor portions of isobutylene oligomers).

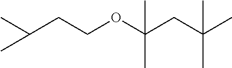

### Application Tests

### 1. Volatility

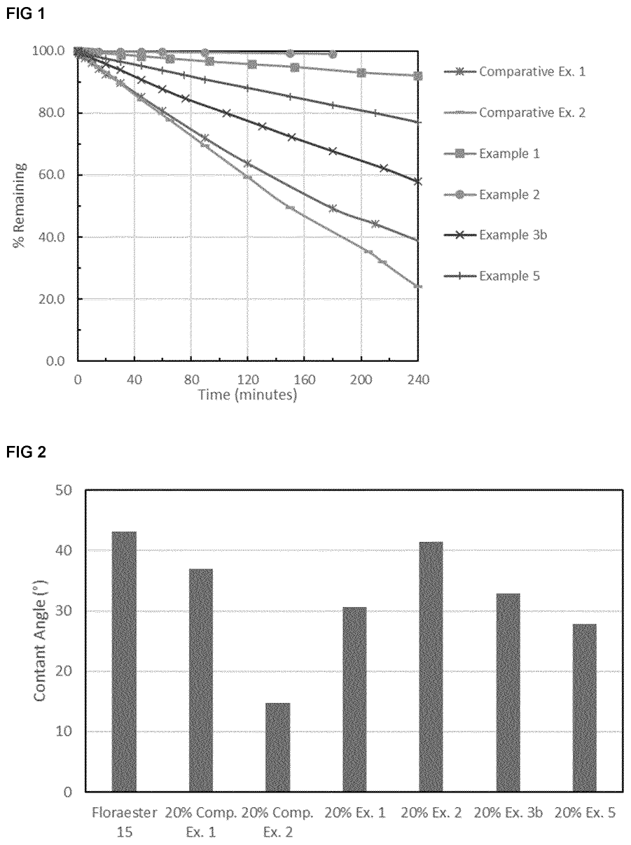

The volatility of the compounds according to the invention and the reference compounds is measured by pouring 2 g of neat material in a 90 mm diameter glass petri dish which is kept on a hot plate of constant temperature of 37° C. The volatility of experimental materials is compared with Comparative Example 1 (Cetiol Ultimate from BASF, INCI Undecane and tridecane) and Comparative Example 2 (SF1202 from Momentive Performance Materials, INCI Cyclomethicone). FIG. 1 shows the amount of oil remaining over 4 h of study.

### 2. Contact Angle

The ability of the material to enhance spreading of cosmetic oils is measured by measuring the contact angle of a mixture comprising 80 wt-% Floraesters15 from Floratech (INCI: Jojoba Esters) and 20 wt-% of the Example compound according to the invention or of the comparative example compound. The lower the contact angle is, the better is the ability of the oil to spread. As the substrate for the contact angle, stripped human forearm skin was chosen.

The substrate of the stripped skin was prepared by pressing sticky side of round disks (Standard D-Squame Disc from Clinical & Derm LLC) on forearm of a volunteer for 5 seconds and removing from skin. The contact angle was measured after 1 minute of placing a drop (˜10 μl) of aforementioned mixture onto the substrate. The contact angle was recorded by Rame-Hart contact angle Advanced Goniometer (Model 300-00-115). The results are displayed in FIG. 2.

### 3. Surface Tension

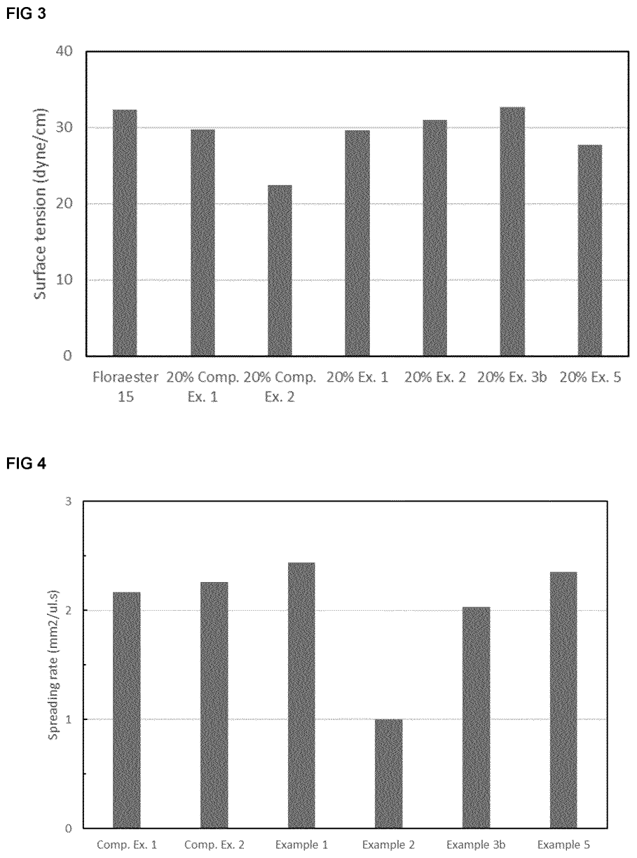

The ability of the material to enhance the spreading of cosmetic oils is also measured by measuring the surface tension of mixture prepared by mixing 80 wt-% Floraesters15 from Floratech (INCI: Jojoba Esters) and 20 wt-% of the Example compound according to the invention or of the comparative example compound. The lower the surface tension is, the better is the ability of the oil to spread. The surface tension is measured by pendant drop method using Rame-Hart Advanced goniometer Goniometer (Model 300-00-115). The results are displayed in FIG. 3.

### 4. Spreading on Forearm

Spreading of the Examples according to the invention and of the comparative examples is measured by dispensing a 10 μl drop onto forearm of 2 volunteers. The drop was allowed to spread for 30 seconds without disturbing the forearm. After 30 seconds, boundaries were marked to note the spreading of the compound. The spreading-area is calculated as, Spreading Area=π×a×b where a and b are major and minor axis of the spreading area. In case the drop spreads spherical then a=b and if the drop spreads elliptical a≠b. The spreading rate of the compounds is then reported as Spreading Area (mm²) per μl per second. Higher the spreading rate, better will be the spreading of the compound on the skin. The spreading rate is displayed in FIG. 4.

### 5. Sensory

The sensory performance of materials was tested by 5 panelists by rubbing small amount of neat Example compounds or neat Comparative Example compounds on the forearm. The performance was measured for spreading during rub, soft after feel, and residue left after rub.

Spreading: 5 out of 5 panelists mentioned enhanced spreading of Example 1 and Example 2 over Comparative Ex. 2.

Soft after feel: 5 out of 5 panelists mentioned a softer after feel of Example 1 and Example 2 than Comparative Ex. 1.

Residue and greasiness: 3 out of 5 panelists mentioned no residue and greasiness was observed with Example 1 and Example 2.

### FORMULATION EXAMPLES

According to the present invention, the ether compounds as described above are comprised in cosmetic compositions in a specific amount depending on the respective formulation. Preferably, the following amounts of the ether compounds according to the invention are used for the respective cosmetic formulations:

### FORMULATION EXAMPLES

| | Typical range % |
|---|---|
| Eye makeup | |
| Eyebrow pencil | 10-30 |
| Eyeliners | 10-60 |
| Eye shadow | 1-50 |
| Eye lotion | 5-60 |
| Eye make-up remover | 5-85 |
| Mascara | 2-50 |
| Hair Care | |
| Conditioners | 0.1-90 |
| Sprays/aerosol fixatives | 0.1-50 |
| Straighteners | 0.1-50 |
| Shampoos | 0.1-50 |
| Dyes and colors | 0.1-20 |
| Hair oils | 10-80 |
| Makeup | |
| Foundations | 1-60 |
| Makeup base, primer | 5-60 |
| Lipstick | 10-50 |
| Makeup fixatives | 1-50 |
| Personal hygiene products | |
| Deodorants | 2-90 |
| Aftershave lotion | 2-50 |
| Preshave lotions | 2-50 |
| Skin care products | |
| Face and neck lotions | 1-90 |
| Body and hand lotions | 1-80 |
| Moisturizers | 1-80 |
| Night creams and lotions | 1-90 |
| Skin fresheners | 1-60 |
| Sun Care products | |
| Suntan gels, creams | 10-60 |
| Sunscreens | 1-50 |

In the following, formulation examples are provided in order to illustrate the subject of the present invention in a non-limiting manner.

### Formulation Example 1: High Shine Lip Stick

| Phase | Ingredient | INCI Name | | wt-% |
|---|---|---|---|---|
| A | Example 1 | | | 8 |
| | Citmol 320 | Trioctyldodecyl Citrate | Alzo | 25 |
| | | | International | |
| | | | Inc. | |
| B | Ozokerite Wax | Ozokerite Wax | | 17 |
| | Rewopal PIB | Polyisobutene | Evonik | 15 |
| | 1000 | | Industries AG | |
| | | | personal care | |
| | Protachem OP | Octyl Palmitate | Protameen | 8 |
| | | | Chemicals | |
| | Silsoft 034 fluid | Caprylyl Methicone | | 6 |
| | Ohlan Lanolin | Hydroxylated Lanolin | The Lubrizol | 3 |
| | Derivative | | Corporation | |
| C | Caprylic/Capric | Caprylic/Capric | | 2 |
| | Triglycerides | Triglycerides | | |
| | Microease 1132 | Synthetic Wax (and) | | 1 |
| | | Microcrystalline Wax | | |
| D | Coloreze Red | D&C Red 7 Ca Lake | | 15 |
| | Lake Dispersion | (and) Ricinus | | |
| | LJ | Communis (Castor) | | |
| | | Seed Oil | | |

For the preparation of the High Shine Lip Stick, the following procedure was applied:
1. All ingredients of the phase A were mixed until the composition was macroscopically uniform, then the ingredients of phase B were added and the composition was heated to 70° C.
2. In a separate container, the ingredients of phase C were melted and added to the main vessel.
3. The ingredients of phase D were added dropwise. The composition was removed from the heat, and the composition was mixed until it was cooled down.

### Formulation Example 2: High Shine Mascara

| Phase | Ingredient | INCI Name | | wt-% |
|---|---|---|---|---|
| A | Example 2 | | Momentive | 5 |
| | | | Performance | |
| | | | Materials, Inc. | |
| | Silsoft 034 | Caprylyl Methicone | Momentive | 1 |
| | fluid | | Performance | |
| | | | Materials, Inc. | |
| | SilForm INX | Bis-Carboxydecyl | Momentive | 2 |
| | fluid | Dimethicone | Performance | |
| | | | Materials, Inc. | |
| | Beeswax | Beeswax | | 8 |
| | Ozokerite Wax | Ozokerite Wax | | 7 |
| | Microease | Synthetic Wax (and) | | 3 |
| | 1132 | Microcrystalline Wax | | |
| | Stearic Acid | Stearic Acid | | 3 |
| | Carnauba Wax | Carnauba Wax | | 3 |
| | SF1642 wax | C30-45 Alkyl | Momentive | 3 |
| | | Dimethicone | Performance | |
| | | | Materials, Inc. | |
| B | DI water | Water | | 36.1 |
| | Tromethamine | Tromethamine | | 1.5 |
| | Natrosol 250 | Hydroxyethylcellulose | Ashland Inc. | 0.3 |
| | HHR | | | |
| C | DI Water | Water | | 1 |
| | Glydant Plus | DMDM Hydantoin (and) | Lonza | 0.6 |
| | Liquid | Iodopropynyl | | |
| | | Butylcarbamate | | |
| | Germall 115 | Imidazolidinyl Urea | Ashland Inc. | 0.5 |
| D | BBO-11S2 | Iron Oxides (and) | Kobo Products, | 25 |
| | | Triethoxycaprylylsilane | Inc | |

For the preparation of the High Shine Mascara, the following procedure was applied:
1. All ingredients of the phase A were mixed together until the composition was macroscopically uniform, and then the composition was heated to 90° C.
2. The ingredients of phase B were mixed and heated to 90° C.
3. Phase A was slowly added to phase B with mixing. The resulting composition was mixed until uniform, then it was removed from the heat.
4. The ingredients of phase C were mixed and added into the main vessel at 40° C.
5. The ingredients of phase 0 were added, and the composition was mixed until it became homogenous.

### Formulation Example 3: BE Cream

| Phase | Ingredient | INCI Name | | wt-% |
|---|---|---|---|---|
| A | Velvesil Mul-T Gel | Caprylyl Methicone (and) C30-45 | Momentive | 7 |
| | | Alkyl Cetearyl Dimethicone | Performance | |
| | | Crosspolymer (and) Boron | Materials, Inc. | |
| | | Nitride | | |
| | Example 2 | Dimethicone | Momentive | 20 |
| | | | Performance | |
| | | | Materials, Inc. | |
| | SilForm FR-5 | Trifluoropropyldimethylsiloxy/ | Momentive | 2 |
| | | Trimethylsiloxy Silsesquioxane | Performance | |
| | | (and) Dimethicone | Materials, Inc. | |
| | SilForm 60-A | PEG/PPG-20/15 Dimethicone | Momentive | 7 |
| | | (and) Diisopropyl Adipate | Performance | |
| | | | Materials, Inc. | |
| | Euxyl PE 9010 | Phenoxyethanol (and) | Schulke & Mayr | 1 |
| | | Ethylhexylglycerin | GmbH | |
| | Fragrance | Fragrance | | 0.2 |
| | Silsoft 034 | Caprylyl Methicone | Momentive | 5 |
| | | | Performance | |
| | | | Materials, Inc. | |
| B | Titanium Dioxide - | Titanium Dioxide (and) | Kobo Products, | 2 |
| | BTD-11S2 | Triethoxycaprylylsilane | Inc | |
| | Yellow Iron Oxide - | Iron Oxide (and) | Kobo Products, | 0.5 |
| | BYO-11S2 | Triethoxycaprylylsilane | Inc | |
| | Red Iron Oxide - | Iron Oxide (and) | Kobo Products, | 0.2 |
| | BRO-11S2 | Triethoxycaprylylsilane | Inc | |
| | Black Iron Oxide - | Iron Oxide (and) | Kobo Products, | 0.05 |
| | BB0-11S2 | Triethoxycaprylylsilane | Inc | |
| | Titanium Dioxide - | Titanium Dioxide (and) Alumina | Kobo Products, | 7 |
| | JTTO-MS7 | (and) Methicone | Inc | |
| | Deionized Water | Water | | 37.15 |
| C | Trisodium EDTA | Trisodium EDTA | | 0.2 |
| | 1,3 Butylene Glycol | Butylene Glycol | | 3 |
| | Glycerin | Glycerin | | 5 |
| | Sodium Chloride | Sodium Chloride | | 1 |
| | Symcalmin | Hydroxyphenyl | Symrise AG | 1 |
| | | Propamidobenzoic Acid | | |
| | Bentone 38V | Disteardimonium Hectorite | Elementis | 0.7 |
| | | | Specialites | |

For the preparation of the BE Cream, the following procedure was applied:
1. The ingredients of phase A were combined in the main vessel and mixed until the composition was macroscopically uniform.
2. The ingredients of phase B were added to the main vessel and homogenized until they were fully dispersed.
3. The ingredients of phase C were combined in a separate vessel and mix until the composition was macroscopically uniform.
4. The ingredients of phase C were slowly added into the main vessel under high-speed agitation. The composition was mixed until it was macroscopically completely uniform.
5. The composition was homogenized until it was uniform.

### Formulation Example 4: Sun Mousse

| Phase | Ingredient | INCI Name | | wt-% |
|---|---|---|---|---|
| A | Velvesil | Cyclopentasiloxane (and) | Momentive | 51 |
| | PLUS | Cetearyl Dimethicone | Performance | |
| | | Crosspolymer (and) | Materials, Inc. | |
| | | PEG/PPG-20/23 | | |
| | | Dimethicone | | |
| | Silsoft 034 | Caprylyl Methicone | Momentive | 13 |
| | | | Performance | |
| | | | Materials, Inc. | |
| | Example 3 | | Momentive | 19 |
| | | | Performance | |
| | | | Materials, Inc. | |
| | SR 1000 | Trimethylsiloxysilicate | Momentive | 2 |
| | | | Performance | |
| | | | Materials, Inc. | |
| | Pigments | Titanium Dioxide | Momentive | 15 |
| | | | Performance | |
| | | | Materials, Inc. | |

For the preparation of the Sun Mousse, the following procedure was applied:
1. All ingredients were mixed in the given order.

### Formulation Example 5: Long Wear Eyeliner

| Phase | Ingredient | INCI Name | | wt-% |
|---|---|---|---|---|
| A | Dermol 99 | Isononyl Isonononoate | Alzo | 22 |
| | | | International, | |
| | | | Inc | |
| | SR1000 | Trimethylsiloxysilicate | Momentive | 6 |
| | | | Performance | |
| | | | Materials, Inc. | |
| | Silform FR-5 | Trifluoropropyl- | Momentive | 7 |
| | Fluid | dimethylsiloxy/ | Performance | |
| | | Trimethylsiloxy | Materials, Inc. | |
| | | Silsesquioxane (and) | | |
| | | Dimethicone | | |
| | Example 1 | | | 20 |
| | KTZ Misterioso | Mica (and) Titanium | Kobo Products, | 3 |
| | Pewter | Dioxide (and) Iron | Inc. | |
| | | Oxide | | |
| | Black Iron | Iron Oxide (and) | Kobo Products, | 12 |
| | Oxide - | Triethoxy- | Inc. | |
| | BB0-11S2 | caprylylsilane | | |
| B | Performalene 500 | Polyethylene | Baker Petrolite, | 4.5 |
| | | | Inc | |
| | Microease 1132 | Synthetic Wax (and) | Micro | 4.5 |
| | | Microcrystalline Wax | Powders, Inc. | |
| | Optiphen | Phenoxyethanol (and) | International | 1 |
| | | Caprylyl Glycol | Specialty | |
| | | | Products, Inc | |
| C | Silsoft ETS | Ethyl Trisiloxane | Momentive | 20 |
| | | | Performance | |
| | | | Materials, Inc. | |

For the preparation of the Long Wear Eyeliner, the following procedure was applied:
1. The ingredients of phase A were mixed until SR1000 and the pigments were completely dispersed in dimethicone. The batch was heated to 90-95° C.
2. The waxes of phase B were added while maintaining heat and agitation until the mixture became uniform.
3. The batch was cooled to 60-65° C., and then Phase C was added. Once the mixture became uniform, it was poured into appropriate packaging.

### Formulation Example 6: Eye Serum

| Phase | Ingredients | INCI Name | wt-% |
|---|---|---|---|
| A | Water | Aqua | up to |
| | | | 100 |
| | Glycerin | Glycerin | 3 |
| | HYDROVANCE | Hydroxyethyl Urea | 3 |
| | Moisturizing | | |
| | Agent | | |
| | Disodium EDTA | Disodium EDTA | 0.05 |
| B | HELIOFEEL | Glyceryl Stearate Citrate (and) | 4 |
| | | Polyglyceryl-3 Stearate (and) | |
| | | Hydrogenated Lecithin | |
| | Lecigel | Sodium Acrylates Copolymer (and) | 1 |
| | | Lecithin | |
| | Cetiol CC4 | Dicaprylyl Carbonate | 2 |
| | Cetiol 868 | Ethylhexyl Stearate | 3 |
| | Avocado Oil | Avocado (Persea Gratissima) Oil | 2 |
| | Shea butter | Butyrospermum Parkii (Shea Butter) | 2 |
| | BHT | BHT | 0.1 |
| | Example 2 | | 3 |
| | Silsoft 034 fluid | Caprylyl Methicone | 2 |
| C | Silsoft E-Pearl | Water (and) Polymethylsilsesquioxane | 3 |
| | PMF emulsion | (and) Dimethicone (and) Isohexadecane | |
| | | (and) Cetearyl Methicone (and) PEG-40 | |
| | | Stearate (and) Steareth-2 (and) | |
| | | Steareth-21 | |
| | Moist 24 | Imperata Cylindrica (Root Extract) | 1 |
| | | (and) Aqua (water) (and) Glycerin (and) | |
| | | PEG 8 (and) Carbomer | |

For the preparation of the Eye Serum, the following procedure was applied:
1. Mix all ingredients of Phase B and heat to 70° C.
2. Heat Phase A to 70° C.
3. Add Phase B to Phase A under intense stirring at 500 rpm. Homogenize mixture for 2 minutes at 10000 rpm.
4. Cool down the mixture to 50° C. with gentle stirring.
5. Add ingredients of Phase C and cool to room temperature.

### Formulation Example 7: Night Cream

| Phase | Ingredients | INCI Name | Wt (%) |
|---|---|---|---|
| A | Water | Aqua | qs to |
| | | | 100 |
| | Keltrol CG-SFT | Xanthan Gum | 0.07 |
| | Glycerin | Glycerin | 3 |
| | Disodium EDTA | Disodium EDTA | 0.05 |
| B | Olivem 1000 | Cetearyl Olivate (and) Sorbitan | 3 |
| | | Olivate | |
| | Olivem VS Feel | Cetearyl Alcohol (and) Cetyl | 2.5 |
| | | Palmitate (and) Sorbitan Palmitate | |
| | | (and) Sorbitan Oleate | |
| | SF 1632 | Cetearyl Methicone | 1 |
| | Cetiol SB 45 | Butyrospermum Parkii (Shea) | 2 |
| | | Butter | |
| | Apricot Kernel | Prunus Armeniaca (Apricot) | 2 |
| | Oil | Kernel Oil | |
| | Arlamol PS15E-LQ | PPG-15 Stearyl Ether | 1 |
| | Miglyol 8810 | Butylene Glycol | 2 |
| | Cetiol 8684 | Ethylhexyl Stearate | 2 |
| | BHT | BHT | 0.1 |
| | Cholesterol USP5 | Cholesterol | 0.1 |
| | Velvesil DM gel | Dimethicone (and) Cetearyl | 2 |
| | | Dimethicone Crosspolymer | |
| | Example 2 | | 2 |
| | Silsoft* 034 fluid | Caprylyl Methicone | 3 |
| C | Carbopol Ultrez 10 | Carbomer | 0.1 |
| | polymer | | |
| | Water | Aqua | 5 |
| D | SYN-COLL | Palmitoyl Tripeptide-5 (and) | 1 |
| | | Glycerin (and) Aqua | |

For the preparation of the Night Cream, the following procedure was applied:
1. All the ingredients of the water phase A were mixed and heated to 70° C.
2. All the ingredients of the oil phase B were mixed and heated to 70° C.
3. The phase B was added to the phase A with intensive stirring. The mixture was homogenized for 1-2 mi (ULTRA-TURRAX†-9500 rpm).
4. Then, the mixture was cooled to 50° C. with gentle stirring.
5. The solution of Carbopol Ultrez 10 polymer in water was added, mixed for 5 min and the pH was adjusted to 6.0-7.0 with base.
   The ingredients of phase 0 were added and mixed until the composition became uniform. The composition was cooled to room temperature.

### Formulation Example 8: Antiperspirant Stick

| Phase | Ingredient | INCI | Wt. % |
|---|---|---|---|
| A | Stearyl alcohol | | 14.0 |
| | Hydrogenated castor oil | | 6.7 |
| | Hallstar PEG 400 DS | PEG-8 distearate | 1.5 |
| | Zirkonal AP4G | Aluminium Zirconium | 24.0 |
| | | Tetrachlorohydrex Gly | |
| B | Example 1 | | 52.8 |

For the preparation of the Antiperspirant Stick, the following procedure was applied:
1. The ingredients of Phase 1 were mixed and the composition was heated to 80-90° C. The composition was mixed until a uniform mixture was formed.
2. Phase B was added mixing was continued for 10 minutes. The resulting composition was cooled to form a solid stick.

### Formulation Example 9: Deodorant Spray

| Phase | Ingredient | Wt. % |
|---|---|---|
| A | Example 2 | 3.0 |
| | C12-C15 alkyl benzoate | 4.5 |
| | Triclosan | 0.3 |
| | Isobutene/propane (80/20) | 92.3 |

For the preparation of the Deodorant Spray, the following procedure was applied:
1. The ingredients of Phase A except Isobutene/propane were mixed and heated to 80-90° C. The composition was mixed until a uniform mixture was formed and cooled to room temperature.
2. Fill the container and charge with propellant isobutene/propane.

### Formulation Example 10: Miracle Hair Oil

| Phase | Ingredient | INCI Name | Wt % |
|---|---|---|---|
| A | Example 20 | | 63.2 |
| | Silsoft 8812¹ | Dimethicone (and) Dimethiconol | 30 |
| | Argan Oil² | Argania Spinosa Kernel Oil | 5 |
| | SF1550¹ | Phenyltrimethicone | 1 |
| | SunShine Super | Synthetic Fluorphlogopite (and) | 0.1 |
| | Gold³ | Titanium Dioxide | |
| | Fragrance⁴ | Perfume | 0.2 |
| | Euxyl PE 9010⁵ | Phenoxyethanol (and) | 0.5 |
| | | Ethylhexylglycerin | |

### Procedure:

1. All ingredients were mixed in consecutive order.
2. The formulation were shaken before use.

### Suppliers:

1. Momentive Performance Materials GmbH
2. DSM
3. Sun Chemical
4. Symrise
5. Schülke & Mayr

The typical use level of the ether compounds according to the invention, in particular of the compound of example 20, in hair oil hair care products is 10-80 wt-%.

### Formulation Example 11: After Sun Tanning Lotion

| Phase | Ingredient | INCI Name | Wt % |
|---|---|---|---|
| A | Softouch* CCS102J¹ | Boron Nitride | 6.00 |
| | SilForm* Flexible Resin¹ | Polymethylsilsesquioxane | 0.50 |
| | Silsoft 1540¹ | Cyclopentasiloxane (and) | 5.00 |
| | | PEG/PPG-20/15 Dimethicone | |
| | Example 21 | | 14.50 |
| | SF1550¹ | Phenyltrimethicone | 1 |
| B | Deionized Water | Water | 66.00 |
| | Glycerin | Glycerin | 3.00 |
| | Sodium Chloride | Sodium Chloride | 1.00 |

### Procedure:

1. SilForm Flexible Resin was dissolved in the ether compound of Example 21.
2. All ingredients of Phase A were added and mixed until uniform.
3. In a separate vessel, all ingredients of Phase B were mixed until a clear solution was formed.
4. Very slowly the Phase B ingredients were added into Phase A while mixing with high-speed agitation.

### Suppliers:

1. Momentive Performance Materials, Inc.

The typical use level of the ether compounds according to the invention, in particular of the compound of example 21, in after sun tanning lotion skin care products is 10-60 wt-%.

### Formulation Example 12: Two Phase Leave-In Hair Conditioner Spray

| Phase | Ingredient | INCI Name | Wt % |
|---|---|---|---|
| A | Water | Water | Q.S. |
| | Covaquat 16 | Polyquaternium-6 | 1.00 |
| | Kathon CG | Cyclopentasiloxane (and) | 0.05 |
| | | PEG/PPG-20/15 Dimethicone | |
| B | Example 17 | | 20.00 |
| | SF-1214 | Dimethicone | 3.00 |
| | Solubilisant LRI | PPG-26 Buteth-26 and PEG-40 | 2.00 |
| | | Hydrogenated Castor Oil PPG-26 | |
| | | Buteth-26/PEG-40 | |
| | Sodium Chloride | Sodium Chloride | 1.0 |

### Procedure:

1. Phase A was mixed under stirring, until completely dissolved.
2. (5) & (6) in Phase B were premixed, then (7) was added with stirring.
3. Phase A was agitated at high to moderate speed, Part B was added slowly to Part A.
4. The agitation speed was reduced and NaCl was added.
5. It was mixed for additional 1 minute.

The typical use level of the ether compounds according to the invention, in particular of the compound of example 17, in two phase leave-in hair conditioner spray is 10-60 wt-%.

### Formulation Example 13: Makeup Removal Gel Cream

| Phase | Ingredient | INCI Name | Wt % |
|---|---|---|---|
| A | Silsoft* 034 fluid | Caprylyl Methicone | 8.00 |
| | Example 19 | | 8.00 |
| | Silsoft 440 fluid | PEG-20/PPG-23 Dimethicone | 3.5 |
| B | Demineralized Water | Water | Q.S. |
| | Carbopol Ultrez 2 | Carbomer | 0.38 |
| C | Velvesil E-Gel PMF | Dimethicone/Vinyl Dimethicone | 3.5 |
| | emulsion | Crosspolymer (and) Dimethicone | |
| | | (and) Isohexadecane (and) | |
| | | Cetearyl Methicone | |
| D | Triethanolamine | Triethanolamine | 0.01 |

### Procedure:

1. Both phase A and phase B were prepared separately.
2. Phases A and B were mixed with short time homogenization until all ingredients were well dispersed.
3. The TEA was added to the phase A/B mixture to adjust the pH level.
4. Preservative and fragrance as needed were added.

The typical use level of the ether compounds according to the invention, in particular of the compound of example 19, in makeup removal gel cream is 5-60 wt-%.

1-27. (canceled)

28. A cosmetic composition comprising at least one compound of the formula (I):
R¹—O—R² (I)
wherein
R¹ and R² are different from each other and are independently selected from the group of C1-C17 hydrocarbyl residues,
said hydrocarbyl residues contain up to 8 CH₃ groups, and wherein
the total number of carbon atoms of said compound is 10 to 20,
the total number of ether groups of said compound is 1,
the total number of methyl groups (—CH₃) of said compound is 1 to 13,
at least one of R¹ and R² is a branched C3-C17 hydrocarbyl residue,
with the proviso for all monoether compounds containing a total number of 10 to 13 carbon atoms the compounds are selected from the group consisting of n-pentyl-1,1,3,3-tetramethyl-butyl ether, cyclopentyl 1,1,3,3-tetramethyl-butyl ether, n-butyl-1,1,3,3-tetramethyl-butyl ether, isoamyl-1,1,3,3-tetramethyl-butyl ether, isobutyl-1,1,3,3-tetramethyl-butyl ether, 3,5,5-trimethyl-hexyl methyl ether, 3,5,5-trimethyl-hexyl ethyl ether, 3,5,5-trimethyl-hexyl n-propyl ether, 3,5,5-trimethyl-hexyl isopropyl ether, 3,5,5-trimethyl-hexyl n-butyl ether, 3,5,5-trimethyl-hexyl 2-butyl ether, 3,5,5-trimethyl-hexyl isobutyl ether and 3,5,5-trimethyl-hexyl tert-butyl ether, 1,1,3,3,5,5-hexamethylhexyl-methyl ether, 2-methylphenyl isopropyl ether, 3-methylphenyl isopropyl ether, 4-methylphenyl isopropyl ether, benzyl isopropyl ether, 3,5,5-trimethyl-hexyl 2-methyl-propyl ether, cyclohexyl 2-methyl-propyl ether, phenyl 2-methyl-propyl ether, 2-methylphenyl 2-methyl-propyl ether, 2-methylphenyl 2-methyl-propyl ether, 4-methylphenyl 2-methyl-propyl ether, benzyl 2-methyl-propyl ether, 3-methyl-butyl cyclopentyl ether, 3-methyl-butyl cyclohexyl ether, 3-methyl-butyl phenyl ether, 2-methylphenyl 3-methyl-butyl ether, 3-methylphenyl 3-methyl-butyl ether, 4-methylphenyl 3-methyl-butyl ether, benzyl 3-methyl-butyl ether, 1,1,3,3-tetramethyl-butyl 3-methyl-butyl ether, cyclohexyl tert-butyl ether, phenyl tert-butyl ether, 2-methylphenyl tert-butyl ether, 3-methylphenyl tert-butyl ether, 4-methylphenyl tert-butyl ether, cyclopentyl 1,1-dimethyl-propyl ether, cyclohexyl 1,1-dimethyl-propyl ether, phenyl 1,1-dimethyl-propyl ether, 2-methylphenyl 1,1-dimethyl-propyl ether, 3-methylphenyl 1,1-dimethyl-propyl ether, 4-methylphenyl 1,1-dimethyl-propyl ether, benzyl 1,1-dimethyl-propyl ether, cyclopentyl 2,2-dimethyl-propyl ether, cyclohexyl 2,2-dimethyl-propyl ether, phenyl 2,2-dimethyl-propyl ether, 2-methylphenyl 2,2-dimethyl-propyl ether, 3-methylphenyl 2,2-dimethyl-propyl ether, 4-methylphenyl 2,2-dimethyl-propyl ether, benzyl 2,2-dimethyl-propyl ether, cyclopentyl 3-methyl-pentyl ether, cyclohexyl 3-methyl-pentyl ether, phenyl 3-methyl-pentyl ether, 2-methylphenyl 3-methyl-pentyl ether, 3-methylphenyl 3-methyl-pentyl ether, 4-methylphenyl 3-methyl-pentyl ether, benzyl 3-methyl-pentyl ether, cyclopentyl 3,3-dimethyl-butyl ether, cyclohexyl 3,3-dimethyl-butyl ether, phenyl 3,3-dimethyl-butyl ether, 2-methylphenyl 3,3-dimethyl-butyl ether, 3-methylphenyl 3,3-dimethyl-butyl ether, 4-methylphenyl 3,3-dimethyl-butyl ether, benzyl 3,3-dimethyl-butyl ether, cyclopentyl 2,3-dimethyl-butyl ether, cyclohexyl 2,3-dimethyl-butyl ether, phenyl 2,3-dimethyl-butyl ether, 2-methylphenyl 2,3-dimethyl-butyl ether, 3-methylphenyl 2,3-dimethyl-butyl ether, 4-methylphenyl 2,3-dimethyl-butyl ether, benzyl 2,3-dimethyl-butyl ether, cyclopentyl 1,3-dimethyl-butyl ether, cyclohexyl 1,3-dimethyl-butyl ether, phenyl 1,3-dimethyl-butyl ether, 2-methylphenyl 1,3-dimethyl-butyl ether, 3-methylphenyl 1,3-dimethyl-butyl ether, 4-methylphenyl 1,3-dimethyl-butyl ether, and
the monoether compounds containing a total number of 14 to 20 carbon atoms are selected from the group consisting of n-hexyl 1,1,3,3-tetramethyl-butyl ether, n-heptyl 1,1,3,3-tetramethyl-butyl ether, n-octyl 1,1,3,3-tetramethyl-butyl ether, n-nonyl 1,1,3,3-tetramethyl-butyl ether, n-decyl 1,1,3,3-tetramethyl-butyl ether, n-undecyl 1,1,3,3-tetramethyl-butyl ether, n-dodecyl 1,1,3,3-tetramethyl-butyl ether, 3-methyl-pentyl 1,1,3,3-tetramethyl-butyl ether, 3,3-dimethyl-butyl 1,1,3,3-tetramethyl-butyl ether, 2,3-dimethyl-butyl 1,1,3,3-tetramethyl-butyl ether, 1,3-dimethyl-butyl 1,1,3,3-tetramethyl-butyl ether, 2,4,4-trimethyl-pentyl 1,1,3,3-tetramethyl-butyl ether, 3,5,5-trimethyl-hexyl 1,1,3,3-tetramethyl-butyl ether, 2-ethyl-hexyl 1,1,3,3-tetramethyl-butyl ether, cyclohexyl 1,1,3,3-tetramethyl-butyl ether, phenyl 1,1,3,3-tetramethyl-butyl ether, 2-methylphenyl 1,1,3,3-tetramethyl-butyl ether, 3-methylphenyl 1,1,3,3-tetramethyl-butyl ether, 4-methylphenyl 1,1,3,3-tetramethyl-butyl ether, benzyl 1,1,3,3-tetramethyl-butyl ether, n-pentyl 1,1,3,3,5,5-hexamethyl-hexyl ether, cyclopentyl 1,1,3,3,5,5-hexamethyl-hexyl ether, cyclohexyl 1,1,3,3,5,5-hexamethyl-hexyl ether, phenyl 1,1,3,3,5,5-hexamethyl-hexyl ether, 2-methylphenyl 1,1,3,3,5,5-hexamethyl-hexyl ether, 3-methylphenyl 1,1,3,3,5,5-hexamethyl-hexyl ether, 4-methylphenyl 1,1,3,3,5,5-hexamethyl-hexyl ether, benzyl 1,1,3,3,5,5-hexamethyl-hexyl ether, 1,1,3,3,5,5-hexamethylhexyl ethyl ether, 1,1,3,3,5,5-hexamethylhexyl-1-propyl ether, 1,1,3,3,5,5-hexamethylhexyl-n-propyl ether, 1,1,3,3,5,5-hexamethylhexyl-n-butyl ether, 1,1,3,3,5,5-hexamethylhexyl-1-butyl ether, 1,1,3,3,5,5-hexamethylhexyl-t-butyl ether, 3,5,5-trimethyl-hexyl n-pentyl ether, 3,5,5-trimethyl-hexyl n-hexyl ether, 3,5,5-trimethyl-hexyl n-heptyl ether, 3,5,5-trimethyl-hexyl n-octyl ether, 3,5,5-trimethyl-hexyl n-nonyl ether,
3,5,5-trimethyl-hexyl 3-methyl-butyl ether, 3,5,5-trimethyl-hexyl 1,1-dimethylpropyl ether, 3,5,5-trimethyl-hexyl 2,2-dimethylpropyl ether, 3,5,5-trimethyl-hexyl 3-methyl-pentyl ether, 3,5,5-trimethyl-hexyl 3,3-dimethyl-butyl ether, 3,5,5-trimethyl-hexyl 2,3-dimethyl-butyl ether, 3,5,5-trimethyl-hexyl 1,3-dimethyl-butyl ether, 3,5,5-trimethyl-hexyl 2,4,4-trimethyl-pentyl ether, 3,5,5-trimethyl-hexyl 2-ethyl-hexyl ether, 3,5,5-trimethyl-hexyl cyclopentyl ether, 3,5,5-trimethyl-hexyl cyclohexyl ether, 3,5,5-trimethyl-hexyl phenyl ether, 3,5,5-trimethyl-hexyl 2-methylphenyl ether, 3,5,5-trimethyl-hexyl 3-methylphenyl ether, 3,5,5-trimethyl-hexyl 4-methylphenyl ether, and 3,5,5-trimethyl-hexyl benzyl ether,
wherein the cosmetic composition contains at least 0.01 weight-% of the compound of the formula (I) based on the total weight of the composition, more preferably 1 to 95 wt-%, even more preferably 2 to 90 weight-%, and most preferably 5 to 80 weight-% based on the total weight of the composition.

29. The cosmetic composition according to claim 28, wherein the monoether compounds containing a total number of 10 to 13 carbon atoms are selected from the group consisting of n-pentyl-1,1,3,3-tetramethyl-butyl ether, cyclopentyl 1,1,3,3-tetramethyl-butyl ether, n-butyl-1,1,3,3-tetramethyl-butyl ether, isoamyl-1,1,3,3-tetramethyl-butyl ether, and isobutyl-1,1,3,3-tetramethyl-butyl ether, and
the monoether compounds containing a total number of 14 to 20 carbon atoms are selected from the group consisting of n-hexyl 1,1,3,3-tetramethyl-butyl ether, n-heptyl 1,1,3,3-tetramethyl-butyl ether, n-octyl 1,1,3,3-tetramethyl-butyl ether, n-nonyl 1,1,3,3-tetramethyl-butyl ether, n-decyl 1,1,3,3-tetramethyl-butyl ether, n-undecyl 1,1,3,3-tetramethyl-butyl ether, n-dodecyl 1,1,3,3-tetramethyl-butyl ether, 3-methyl-pentyl 1,1,3,3-tetramethyl-butyl ether, 3,3-dimethyl-butyl 1,1,3,3-tetramethyl-butyl ether, 2,3-dimethyl-butyl 1,1,3,3-tetramethyl-butyl ether, 1,3-dimethyl-butyl 1,1,3,3-tetramethyl-butyl ether, 2,4,4-trimethyl-pentyl 1,1,3,3-tetramethyl-butyl ether, 3,5,5-trimethyl-hexyl 1,1,3,3-tetramethyl-butyl ether, 2-ethyl-hexyl 1,1,3,3-tetramethyl-butyl ether, cyclohexyl 1,1,3,3-tetramethyl-butyl ether, phenyl 1,1,3,3-tetramethyl-butyl ether, 2-methylphenyl 1,1,3,3-tetramethyl-butyl ether, 3-methylphenyl 1,1,3,3-tetramethyl-butyl ether, 4-methylphenyl 1,1,3,3-tetramethyl-butyl ether, and benzyl 1,1,3,3-tetramethyl-butyl ether.

30. The cosmetic composition according to claim 28, wherein the monoether compound containing a total number of 10 to 13 carbon atoms is from 1,1,3,3,5,5-hexamethylhexyl-methyl ether, and
the monoether compounds containing a total number of 14 to 20 carbon atoms are selected from the group consisting of n-pentyl 1,1,3,3,5,5-hexamethyl-hexyl ether, cyclopentyl 1,1,3,3,5,5-hexamethyl-hexyl ether, cyclohexyl 1,1,3,3,5,5-hexamethyl-hexyl ether, phenyl 1,1,3,3,5,5-hexamethyl-hexyl ether, 2-methylphenyl 1,1,3,3,5,5-hexamethyl-hexyl ether, 3-methylphenyl 1,1,3,3,5,5-hexamethyl-hexyl ether, 4-methylphenyl 1,1,3,3,5,5-hexamethyl-hexyl ether, benzyl 1,1,3,3,5,5-hexamethyl-hexyl ether, 1,1,3,3,5,5-hexamethylhexyl ethyl ether, 1,1,3,3,5,5-hexamethylhexyl-1-propyl ether, 1,1,3,3,5,5-hexamethylhexyl-n-propyl ether, 1,1,3,3,5,5-hexamethylhexyl-n-butyl ether, 1,1,3,3,5,5-hexamethylhexyl-1-butyl ether, and 1,1,3,3,5,5-hexamethylhexyl-t-butyl ether.

31. The cosmetic composition according to claim 28, wherein the monoether compounds containing a total number of 10 to 13 carbon atoms are selected from the group consisting of 3,5,5-trimethyl-hexyl methyl ether, 3,5,5-trimethyl-hexyl ethyl ether, 3,5,5-trimethyl-hexyl n-propyl ether, 3,5,5-trimethyl-hexyl isopropyl ether, 3,5,5-trimethyl-hexyl n-butyl ether, 3,5,5-trimethyl-hexyl 2-butyl ether, 3,5,5-trimethyl-hexyl isobutyl ether and 3,5,5-trimethyl-hexyl tert-butyl ether, and
the monoether compounds containing a total number of 14 to 20 carbon atoms are selected from the group consisting of 3,5,5-trimethyl-hexyl n-pentyl ether, 3,5,5-trimethyl-hexyl n-hexyl ether, 3,5,5-trimethyl-hexyl n-heptyl ether, 3,5,5-trimethyl-hexyl n-octyl ether, 3,5,5-trimethyl-hexyl n-nonyl ether, 3,5,5-trimethyl-hexyl 3-methyl-butyl ether, 3,5,5-trimethyl-hexyl 1,1-dimethylpropyl ether, 3,5,5-trimethyl-hexyl 2,2-dimethylpropyl ether, 3,5,5-trimethyl-hexyl 3-methyl-pentyl ether, 3,5,5-trimethyl-hexyl 3,3-dimethyl-butyl ether, 3,5,5-trimethyl-hexyl 2,3-dimethyl-butyl ether, 3,5,5-trimethyl-hexyl 1,3-dimethyl-butyl ether, 3,5,5-trimethyl-hexyl 2,4,4-trimethyl-pentyl ether, 3,5,5-trimethyl-hexyl 2-ethyl-hexyl ether, 3,5,5-trimethyl-hexyl cyclopentyl ether, 3,5,5-trimethyl-hexyl cyclohexyl ether, 3,5,5-trimethyl-hexyl phenyl ether, 3,5,5-trimethyl-hexyl 2-methylphenyl ether, 3,5,5-trimethyl-hexyl 3-methylphenyl ether, 3,5,5-trimethyl-hexyl 4-methylphenyl ether, and 3,5,5-trimethyl-hexyl benzyl ether.

32. A cosmetic composition according to claim 28, wherein the compound of the formula (I) is selected from the group consisting of: 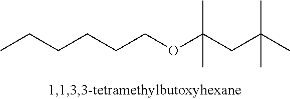
and the further group of mono-ethers consisting of

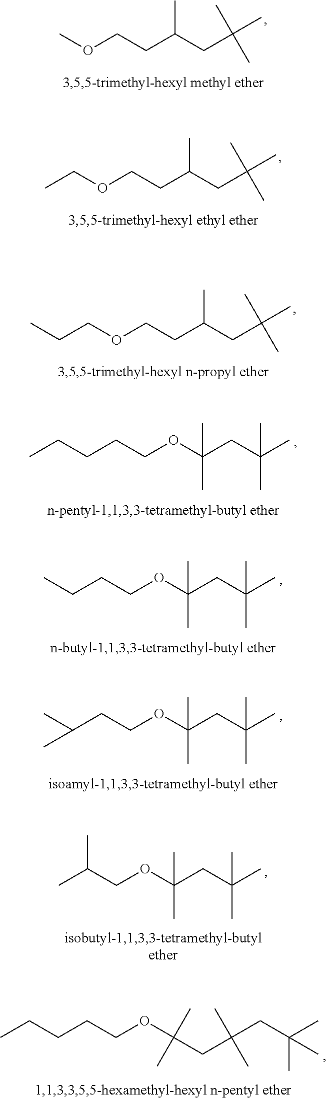

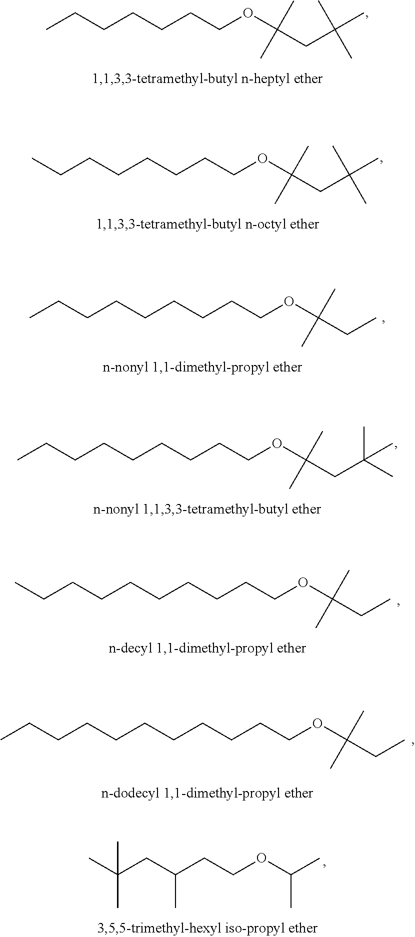

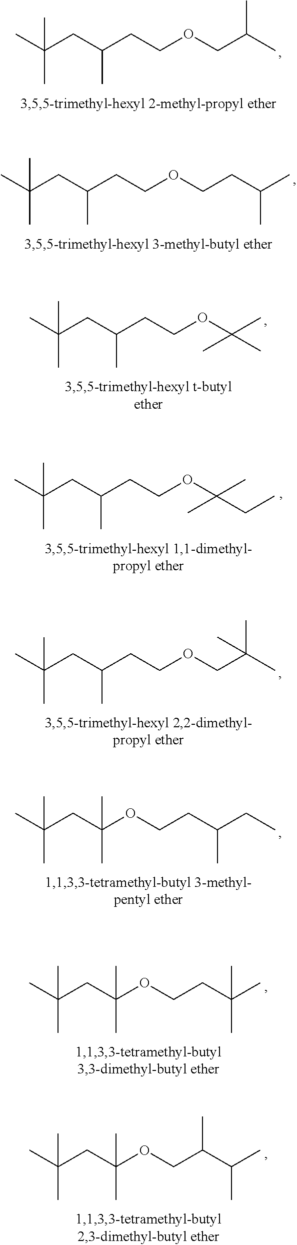

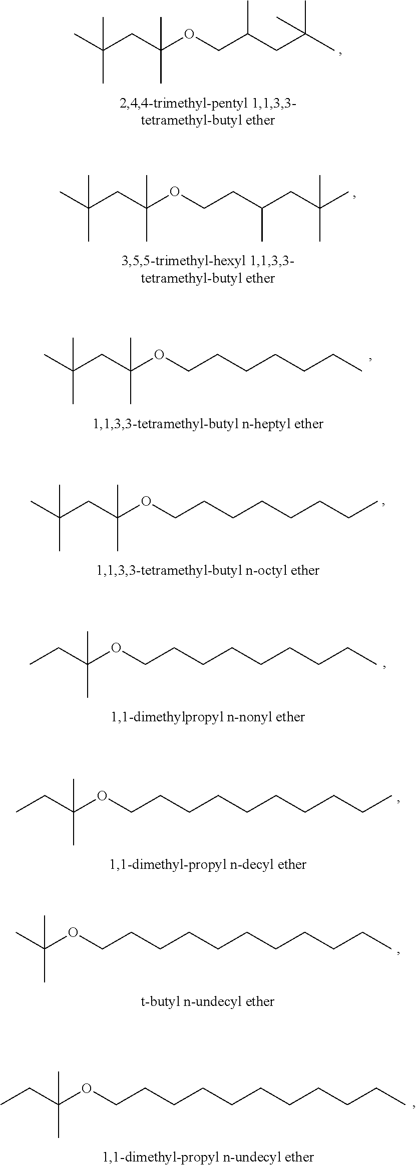

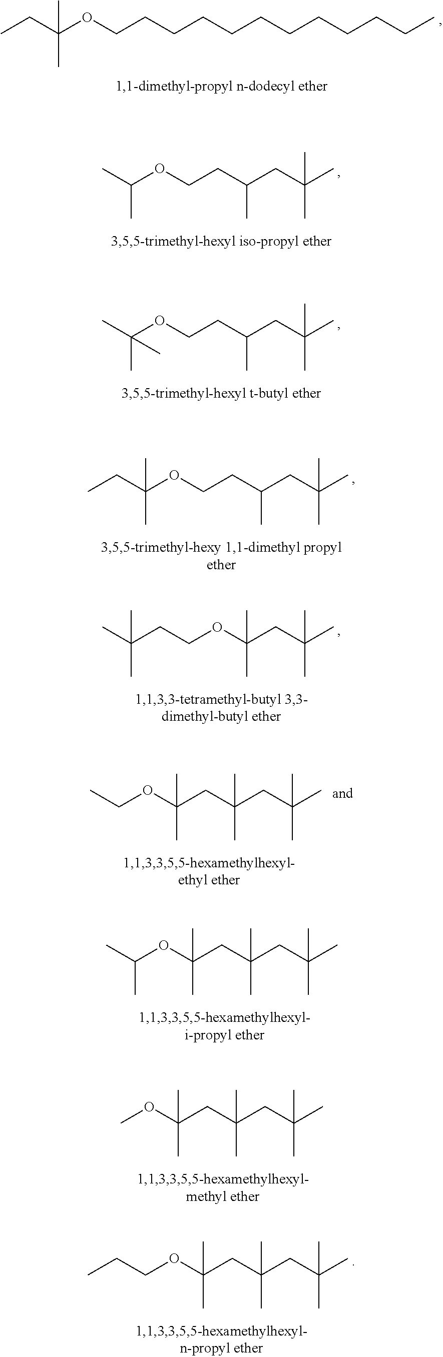

33. A cosmetic composition according to claim 28, wherein the compound of the formula (I) is selected from the group consisting of: 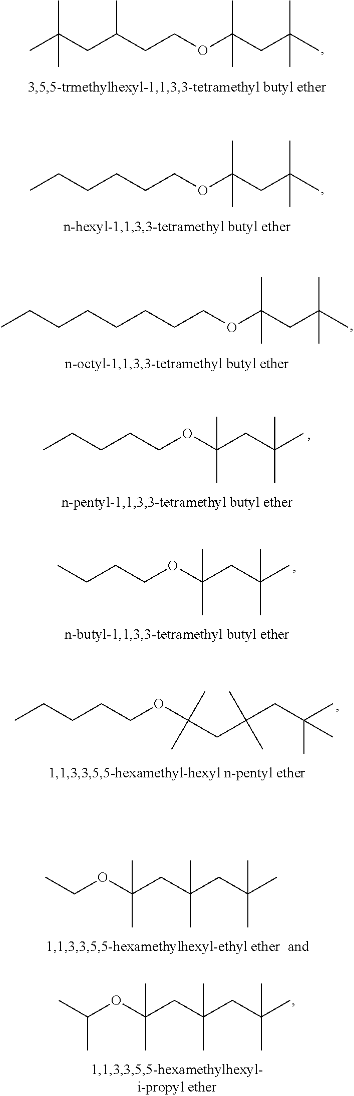
preferably from n-pentyl-1,1,3,3-tetramethyl butyl ether or n-butyl-1,1,3,3-tetramethyl butyl ether.

34. The cosmetic composition according to claim 28, wherein the cosmetic composition is an aqueous composition, and wherein preferably the water content of the cosmetic composition is in the range of 10 to 80 wt-%, more preferably 15 to 70 weight-% of the composition, most preferably 20 to 60 weight-% based on the total weight of the composition.

35. The cosmetic composition according to claim 28, wherein the cosmetic composition is a skin care formulation, preferably selected from a lotion, a cream or an emulsion, more preferably selected from under eye cream, day cream, night cream, and makeup removal gel cream.

36. The cosmetic composition according to claim 35, wherein the cosmetic composition is a skin care formulation comprising 0.1 to 90 wt-%, more preferably 2 to 80 wt-%, even more preferably 5 to 70 wt-%, and most preferably 10 to 60 wt % of the at least one compound according to the formula (I) based on the total weight of the cosmetic composition.

37. The cosmetic composition according to claim 35, wherein the at least one compound of the formula (I) is selected from n-pentyl-1,1,3,3-tetramethyl butyl ether or n-butyl-1,1,3,3-tetramethyl butyl ether.

38. The cosmetic composition according to claim 28, wherein the cosmetic composition is a color cosmetic, preferably selected from lip stick, mascara, bb cream, and eye liner.

39. The cosmetic composition according to claim 38, wherein the cosmetic composition is a color cosmetic comprising 1 to 90 wt-%, more preferably 2 to 80 wt-%, even more preferably 3 to 70 wt-%, and most preferably 5 to 60 wt-% of the at least one compound according to the formula (I) based on the total weight of the cosmetic composition.

40. The cosmetic composition according to claim 38, wherein the at least one compound of the formula (I) is selected from n-pentyl-1,1,3,3-tetramethyl butyl ether or n-butyl-1,1,3,3-tetramethyl butyl ether.

41. The cosmetic composition according to claim 28, wherein the cosmetic composition is a hair care formulation, preferably a hair care formulation selected from hair oils, and hair sprays, in particular leave-in-hair conditioner spray.

42. The cosmetic composition according to claim 41, wherein the composition is a hair care formulation comprising 0.01 to 99 wt-%, more preferably 0.5 to 95 wt-%, even more preferably 1 to 92 wt-%, and most preferably 2 to 90 wt-% of the at least one compound according to the formula (I) based on the total weight of the cosmetic composition.

43. The cosmetic composition according to claim 41, wherein the at least one compound of the formula (I) is selected from n-pentyl-1,1,3,3-tetramethyl butyl ether or n-butyl-1,1-3,3-tetramethyl butyl ether.

44. The cosmetic compositions according to claim 28, comprising at least one compound of the formula (I), preferably selected from n-pentyl-1,1,3,3-tetramethyl butyl ether or n-butyl-1,1,3,3-tetramethyl butyl ether, in the following quantities:

| | Typical range % |
|---|---|
| Eye makeup | |
| Eyebrow pencil | 10-30, preferred 10-20 and 20-30 |
| Eyeliners | 10-60, preferred 10-35 and 35-60 |
| Eye shadow | 1-50, preferred 1-25 and 25-50 |
| Eye lotion | 5-60, preferred 5-30 and 30-60 |
| Eye make-up remover | 5-85, preferred 5-40 and 40-85 |
| Mascara | 2-50, preferred 2-25 and 25-50 |
| Hair Care | |
| Conditioners | 0.1-90, preferred 0.1-45 and 45-90 |
| Sprays/aerosol fixatives | 0.1-50, preferred 0.1-25 and 25-50 |
| Straighteners | 0.1-50, preferred 0.1-25 and 25-50 |
| Shampoos | 0.1-50, preferred 0.1-25 and 25-50 |
| Dyes and colors | 0.1-20, preferred 0.1-10 and 10-20 |
| Hair oils | 10-80, preferred 10-40 and 40-80 |
| Makeup | |
| Foundations | 1-60, preferred 1-30 and 30-60 |
| Makeup base, primer | 5-60, preferred 5-30 and 30-60 |
| Lipstick | 10-50, preferred 10-30 and 30-50 |
| Makeup fixatives | 1-50, preferred 1-25 and 25-50 |
| Personal hygiene products | |
| Deodorants | 2-90, preferred 2-45 and 45-90 |
| Aftershave lotion | 2-50, preferred 2-25 and 25-50 |
| Preshave lotions | 2-50, preferred 2-25 and 25-50 |
| Skin care products | |
| Face and neck lotions | 1-90, preferred 1-45 and 45-90 |
| Body and hand lotions | 1-80, preferred 1-40 and 40-80 |
| Moisturizers | 1-80, preferred 1-40 and 40-80 |
| Night creams and lotions | 1-90, preferred 1-45 and 45-90 |
| Skin fresheners | 1-60, preferred 1-30 and 30-60 |
| Sun Care products | |
| Suntan gels, creams | 10-60, preferred 10-35 and 35-60 |
| Sunscreens | 1-50, preferred 1-25 and 25-50 |

45. The cosmetic composition according to claim 28, wherein the cosmetic composition comprises at least one further ingredient, preferably at least two further ingredients, more preferably at least three further ingredients selected from oils, waxes, thickeners, humectants, sunscreens, emollients, fats obtained from animals, or minerals, in particular metal oxides, organic compounds acting as colorants, fragrances or preservatives, pigments, natural products and mixtures thereof obtained by extraction of plants, processed plant parts or polymers, emulsifiers and surfactants.

46. A compound of the formula (I′):
R¹—O—R² (I′)
wherein
R¹ and R² are different from each other and are independently selected from the group of C1-C17 hydrocarbyl residues,
said hydrocarbyl residues contain up to 8 CH₃ groups and no ether groups (—O—),
the total number of carbon atoms of said compounds is 10 to 20,
the total number of ether groups of said compounds is 1,
the total number of methyl groups (—CH₃) of said compounds is 1 to 13,
at least one of R¹ and R² is a branched C3-C17 hydrocarbyl residue, the one or more compounds are selected from monoethers derived from tri-isobutene, wherein R¹ is a 1,1,3,3,5,5-hexamethylhexyl group, and R² is selected from linear pentyl or hexyl groups or branched C3-C8 alkyl groups, and
the compound 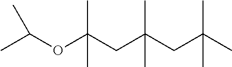 [SMILES: CC(C)OC(C)(C)CC(C)(C)CC(C)(C)C]
is excluded.
